(19) United States
(12) Patent Application Publication
(10) Pub. No.: US 2025/0331705 A1
(43) Pub. Date: Oct. 30, 2025

(54) MEDICAL MANIPULATOR AND MEDICAL MANIPULATOR SYSTEM

(71) Applicant: OLYMPUS CORPORATION, Tokyo (JP)

(72) Inventors: Michael BLOM, Harston (GB); Alex NORMAN, Harston (GB); Mark RIDLEY, Harston (GB); Andrew FORBES, Harston (GB); Merissa LIM, Harston (GB); Antonia SMITH, Harston (GB); Gary STACEY, Harston (GB); Ka Chung LEE, Harston (GB); Wayne GIMBLETT, Harston (GB); Imantha SAMARANAYAKE, Harston (GB); Simon THORNE, Harston (GB); Charles KILBY, Harston (GB); Shintaro INOUE, Westborough, MA (US); Ryohei OGAWA, Hachioji-shi (JP); Yuji SAKAI, Hachioji-shi (JP); Ryoji HYODO, Hachioji-shi (JP); Masahiro FUJII, Hachioji-shi (JP); Shotaro TAKEMOTO, Hachioji-shi (JP); Masato NARISAWA, Hachioji-shi (JP)

(73) Assignee: OLYMPUS CORPORATION, Tokyo (JP)

(21) Appl. No.: 19/074,787

(22) Filed: Mar. 10, 2025

Related U.S. Application Data
(60) Provisional application No. 63/562,855, filed on Mar. 8, 2024.
(60) Provisional application No. 63/635,021, filed on Apr. 17, 2024.
(60) Provisional application No. 63/648,937, filed on May 17, 2024.
(60) Provisional application No. 63/664,879, filed on Jun. 27, 2024.
(60) Provisional application No. 63/669,306, filed on Jul. 10, 2024.
(60) Provisional application No. 63/688,972, filed on Aug. 30, 2024.
(60) Provisional application No. 63/691,009, filed on Sep. 5, 2024.
(60) Provisional application No. 63/695,602, filed on Sep. 17, 2024.
(60) Provisional application No. 63/737,339, filed on Dec. 20, 2024.
(60) Provisional application No. 63/763,398, filed on Feb. 26, 2025.

(51) Int. Cl. A61B 1/005; A61B 1/00; A61B 1/018; A61B 1/05; A61M 25/01

(52) U.S. Cl. CPC A61B 1/0057; A61B 1/00006; A61B 1/00135; A61B 1/0055; A61B 1/018; A61B 1/05; A61M 25/0138; A61M 25/0147

(57) ABSTRACT

A medical manipulator includes: a manipulator; and a camera unit provided at a distal end of the manipulator so as to be movable back and forth in a longitudinal direction, and the camera unit includes a support portion, and a camera provided at a distal end side of the support portion, and the camera unit includes a first state in which the camera unit advances relative to the manipulator, a second state in which the camera unit retracts relative to the manipulator, and a third state in which the camera unit bends downward.

The present disclosure relates to a medical manipulator and a medical manipulator system. The present application claims priority on U.S. Provisional Patent Applications No. 63/562,855 filed on Mar. 8, 2024, No. 63/635,021 filed on Apr. 17, 2024, No. 63/648,937 filed on May 17, 2024, No. 63/664,879 filed on Jun. 27, 2024, No. 63/669,306 filed on Jul. 10, 2024, No. 63/688,972 filed on Aug. 30, 2024, No. 63/691,009 filed on Sep. 5, 2024, No. 63/695,602 filed on Sep. 17, 2024, and No. 63/737,339 filed on Dec. 20, 2024, the contents of which are incorporated herein by reference.

### TECHNICAL FIELD

### Background

Conventionally, a medical manipulator system for observation or treatment in a hollow organ such as the alimentary canal has been used. The medical manipulator system can electrically drive an insertion section or the like inserted into a hollow organ. A user can control the operation of the insertion section or the like using an operation device provided outside of an internal part.

In Patent Document 1, a medical system including an endoscope which is electrically driven is described. In the medical system described in Patent Document 1, since the endoscope is electrically driven, it is possible to reduce fatigue of an operator.

### PRIOR ART DOCUMENTS

### Patent Documents

[Patent document 1] PCT International Publication No. WO 2021/145411

### SUMMARY

A medical manipulator according to a first aspect of the present invention includes: a manipulator; and a camera unit provided at a distal end of the manipulator so as to be movable back and forth in a longitudinal direction, and the camera unit includes a support portion, and a camera provided at a distal end side of the support portion, and the camera unit includes a first state in which the camera unit advances relative to the manipulator, a second state in which the camera unit retracts relative to the manipulator, and a third state in which the camera unit bends downward.

### BRIEF DESCRIPTION OF THE DRAWINGS

FIG. 1 A diagram illustrating the entire configuration of an electric endoscope system according to a first embodiment.

FIG. 2 A diagram illustrating an insertion manipulator of an electric endoscope system inserted into a large intestine.

FIG. 3 A diagram illustrating a distal end portion of an insertion section of the insertion manipulator.

FIG. 4 A front view of the distal end portion when seen from the distal end side.

FIG. 5 A sectional view of the distal end portion.

FIG. 6 A diagram illustrating a bendable portion of the insertion manipulator.

FIG. 7 A diagram illustrating joint rings of the bendable portion.

FIG. 8 A diagram illustrating a first channel tube of the insertion manipulator.

FIG. 9 A sectional view of a proximal end channel tube of the first channel tube.

FIG. 10 A functional block diagram of a drive device.

FIG. 11 A functional block diagram of a video control device.

FIG. 12 A diagram illustrating a modified example of the bendable portion.

FIG. 13 A diagram illustrating a first channel tube inserted into the modified example of the bendable portion.

FIG. 14 A diagram illustrating a modified example of the insertion section.

FIG. 15 A diagram illustrating a spiral tube.

FIG. 16 A diagram illustrating a modified example of the distal end portion.

FIG. 17 A diagram illustrating a hardness-changing device according to a second embodiment.

FIG. 18 A diagram illustrating the entire configuration of the hardness-changing device.

FIG. 19 A diagram illustrating a hardness-changing unit.

FIG. 20 A diagram illustrating the hardness-changing unit.

FIG. 21 A diagram illustrating an operation of the hardness-changing unit.

FIG. 22 A diagram illustrating an operation of the hardness-changing unit.

FIG. 23 A diagram illustrating an operation of the hardness-changing unit.

FIG. 24 A diagram illustrating an operation of the hardness-changing unit.

FIG. 25 A diagram illustrating an operation of the hardness-changing unit.

FIG. 26 A diagram illustrating a modified example of the hardness-changing unit.

FIG. 27 A diagram illustrating the modified example of the hardness-changing unit.

FIG. 28 A diagram illustrating another modified example of the hardness-changing unit.

FIG. 29 A diagram illustrating the modified example of the hardness-changing unit.

FIG. 30 A diagram illustrating another modified example of the hardness-changing unit.

FIG. 31 A diagram illustrating the modified example of the hardness-changing unit.

FIG. 32 A diagram illustrating another modified example of the hardness-changing unit.

FIG. 33 A diagram illustrating a high-frequency knife in a manipulator tool according to a third embodiment.

FIG. 34 A diagram illustrating a high-frequency knife for marking.

FIG. 35 A diagram illustrating a local injection needle.

FIG. 36 A diagram illustrating the local injection needle that locally injects a liquid.

FIG. 37 A diagram illustrating the high-frequency knife for incision.

FIG. 38 A diagram illustrating the high-frequency knife for incision.

FIG. 39 A diagram illustrating a basket.

FIG. 40 A diagram illustrating the basket for recovering a target part.

FIG. 41 A diagram illustrating a manipulator tool according to a fourth embodiment.

FIG. 42 A sectional view of the manipulator tool.

FIG. 43 A diagram illustrating a modified example of the manipulator tool.

FIG. 44 A diagram illustrating an artificial muscle disposed at another position.

FIG. 45 A diagram illustrating an artificial muscle disposed at another position.

FIG. 46 A diagram illustrating an artificial muscle disposed at another position.

FIG. 47 A diagram illustrating an artificial muscle disposed at another position.

FIG. 48 A diagram illustrating an artificial muscle disposed at another position.

FIG. 49 A diagram illustrating another modified example of the manipulator tool.

FIG. 50 An overall view of an electric endoscope system according to a fifth embodiment.

FIG. 51 A perspective view of the distal end of an insertion manipulator of the electric endoscope system.

FIG. 52 A cross-sectional view of the insertion section of the insertion manipulator.

FIG. 53 A view showing the insertion section.

FIG. 54 A view showing the bending portion of the insertion manipulator.

FIG. 55 A view showing a ring member.

FIG. 56 A view showing the ring member as viewed from the longitudinal direction.

FIG. 57 A view showing a first channel tube.

FIG. 58 A view showing a treatment manipulator protruding from a first opening.

FIG. 60 A view showing the treatment manipulator.

FIG. 61 Same as above.

FIG. 62 Perspective view of the bending tube.

FIG. 63 Exploded view of the bending tube expanded in the circumferential direction.

FIG. 64 View of the bending tube compressed.

FIG. 65 View of the bending tube bending.

FIG. 66 View of an artificial muscle.

FIG. 67 View of a bending piece.

FIG. 68 View of a modified bending piece.

FIG. 69 View of another modified bending piece.

FIG. 70 View of another modified bending piece.

FIG. 71 View of another modified bending piece.

FIG. 72 View of a connecting piece.

FIG. 73 View of a treatment instrument arm with a bending second bending portion.

FIG. 74 Functional block diagram of a drive device.

FIG. 75 View of an insertion drive unit.

FIG. 76 View of a drive unit.

FIG. 77 View of the operation of the drive unit.

FIG. 78 Same as above.

FIG. 79 Exploded view of the first drive unit.

FIG. 80 Cross-sectional view of the first motor unit with the first adapter separated.

FIG. 81 Cross-sectional view of the first motor unit with the first adapter attached.

FIG. 82 Exploded view of the second drive unit.

FIG. 83 Same as above.

FIG. 84 Same as above.

FIG. 85 Cross-sectional view of the second motor unit with the second adapter attached.

FIG. 86 Cross-sectional view of the second motor unit with the third adapter and the fourth adapter attached.

FIG. 87 A diagram showing an example of the operation of the drive unit.

FIG. 88 Same as above.

FIG. 89 Same as above.

FIG. 90 Same as above.

FIG. 91 Same as above.

FIG. 92 A diagram showing a rack as a modified example of the cart.

FIG. 93 A diagram showing a scope protruding from the distal end.

FIG. 94 A diagram explaining the operation of the scope operating wire.

### DETAILED DESCRIPTION

### First Embodiment

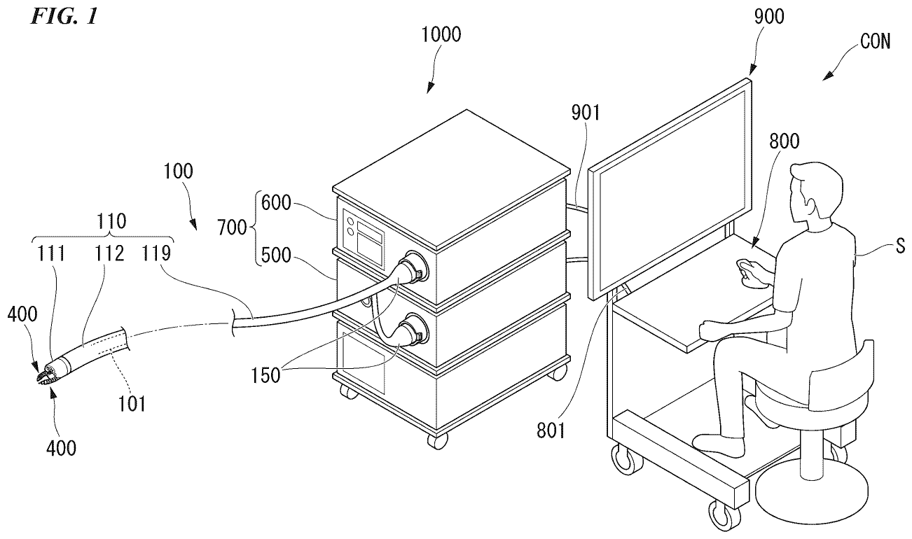

An electric endoscope system 1000 according to a first embodiment of the present invention will be described below with reference to FIGS. 1 to 13. FIG. 1 is a diagram illustrating the entire configuration of the electric endoscope system 1000 according to the present embodiment. The electric endoscope system 1000 is an example of a medical manipulator system. A medical manipulator includes an insertion manipulator 100 inserted into a human body and an endoscope, a catheter, a treatment tool, and an endoluminal device which are electrically driven.

### [Electric Endoscope System 1000]

The electric endoscope system 1000 is a medical system used to observe and treat an internal part of a patient. The electric endoscope system 1000 includes an insertion manipulator 100, a treatment manipulator 400, a drive device 500, a video control device 600, an operation device 800, and a display device 900.

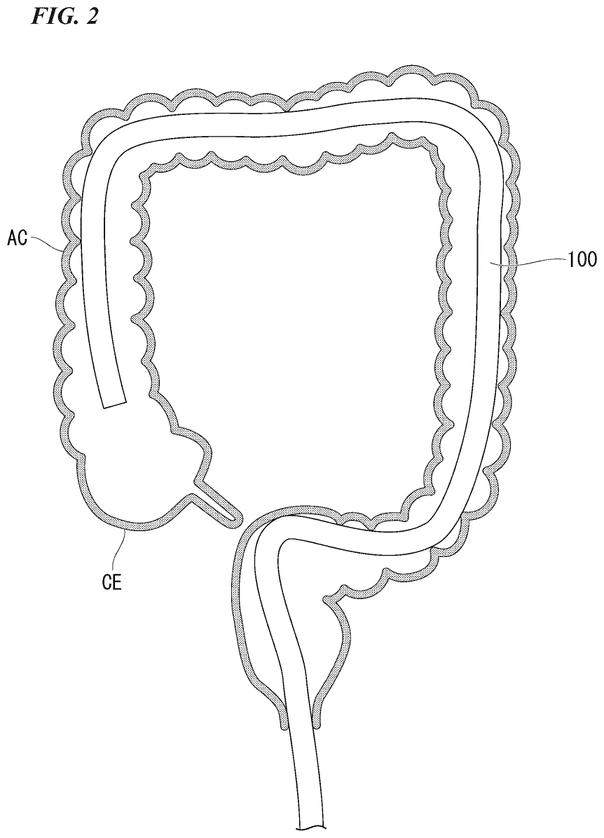

FIG. 2 is a diagram illustrating an insertion manipulator 100 inserted into a large intestine.

The insertion manipulator 100 is a device inserted into a lumen of a patient to observe and treat a lesion. The insertion manipulator 100 has excellent insertability and can be inserted into, for example, an ascending colon AC or a cecum CE of a large intestine as illustrated in FIG. 2. The insertion manipulator 100 is detachably attached to the drive device 500 and the video control device 600. An internal path 101 is formed in the insertion manipulator 100. In the following description, a side of the insertion manipulator 100 inserted into a lumen of a patient is referred to as a “distal end side (distal side) A1,” and a side attached to the drive device 500 is referred to as a “proximal side (proximal side) A2.”

The treatment manipulator 400 is, for example, a device inserted into a first channel tube 171 of the insertion manipulator 100, protrudes from a first opening 111a, and is inserted into a lumen of a patient to treat a lesion. An end effector (a treatment portion) that treats a lesion is disposed at the distal end of the treatment manipulator 400.

The drive device 500 is detachably connected to the insertion manipulator 100 and the operation device 800. The drive device 500 drives a motor built thereinto on the basis of an operation input to the operation device 800 to electrically drive the insertion manipulator 100. The drive device 500 drives a pump or the like built thereinto on the basis of an operation input to the operation device 800 and causes the insertion manipulator 100 to perform air supply, water supply, and suction.

The video control device 600 is detachably connected to the insertion manipulator 100 and acquires a captured image from the insertion manipulator 100. The video control device 600 causes the display device 900 to display the captured image acquired from the insertion manipulator 100 or a GUI image or a CG image to provide information to an operator.

The drive device 500 and the video control device 600 constitute a control device 700 that controls the electric endoscope system 1000. The control device 700 may further include peripherals such as a video printer. The drive device 500 and the video control device 600 may be a unified device.

The operation device 800 is detachably connected to the drive device 500 via an operation cable 801. The operation device 800 may communicate with the drive device 500 in a wireless manner instead of in a wired manner. An operator S can electrically drive the insertion manipulator 100 by operating the operation device 800.

The display device 900 is a device that can display an image such as an LCD. The display device 900 is connected to the video control device 600 via a display cable 901.

The operation device 800 and the display device 900 are mounted in a trolley. The trolley on which the operation device 800 and the display device 900 are mounted is also referred to as a “console CON.”

The constituent devices of the electric endoscope system 1000 will be described below.

### [Insertion Manipulator 100]

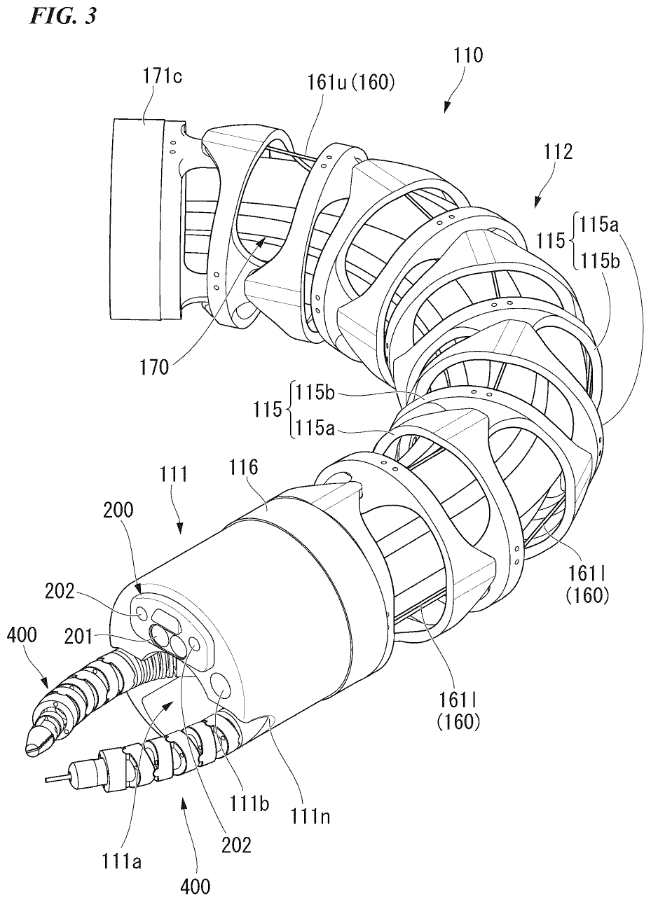

FIG. 3 is a diagram illustrating a distal end portion of an insertion section 110.

The insertion manipulator 100 includes an insertion section 110, an attachment portion 150, a bending wire 160, a built-in element 170, and a scope 200 as illustrated in FIGS. 1 and 3.

In the insertion manipulator 100, an internal path (a lumen) 101 extending in a longitudinal direction (a longitudinal axis direction, an axial direction) A of the insertion manipulator 100 is formed from the distal end of the insertion section 110 to the proximal end of the attachment portion 150. The bending wire 160 and the built-in element 170 are inserted into the internal path 101.

The insertion section 110 is a thin longitudinal member that can be inserted into a lumen. The insertion section 110 includes a distal end portion 111, a bending portion 112, and a flexible portion 119. The distal end portion 111, the bending portion 112, and the flexible portion 119 are sequentially connected from the distal end side A1 to the proximal side A2. The insertion section 110 includes an outer sheath 118 which is the outermost part.

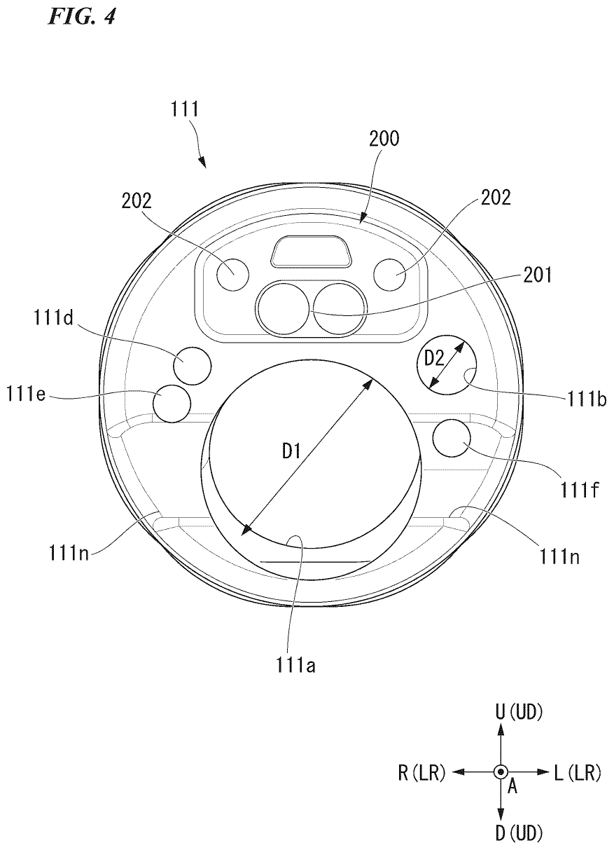

FIG. 4 is a front view of the distal end portion 111 when seen from the distal end side A1.

The distal end portion 111 is formed in a cylindrical shape. The distal end portion 111 includes a first opening 111a, a second opening 111b, a water supply nozzle 111d, an air supply nozzle 111e, and a suction nozzle 111f. The first opening 111a, the second opening 111b, the water supply nozzle 111d, the air supply nozzle 111e, and the suction nozzle 111f are formed on a distal end surface of the distal end portion 111.

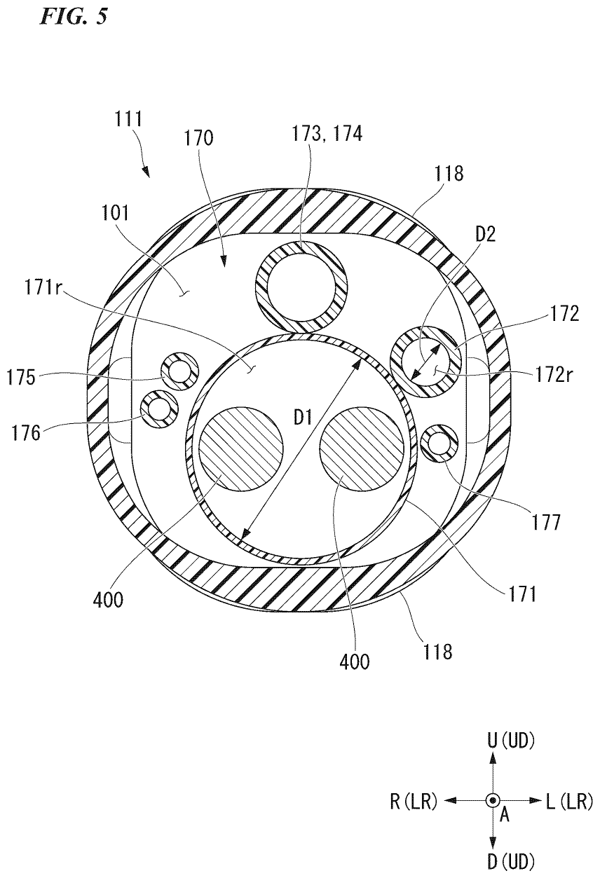

FIG. 5 is a sectional view of the distal end portion 111.

The built-in element 170 is inserted into the internal path 101. The built-in element 170 includes a first channel tube 171, a second channel tube 172, an imaging cable 173, a light guide 174, a water supply tube 175, an air supply tube 176, and a suction tube 177. In FIG. 5, two treatment manipulators 400 that are inserted into the first channel tube 171 are also illustrated.

The first opening 111a is an opening that communicates with the first channel tube 171. The first opening 111a is a circular opening in a front view when seen from the distal end side A1. The distal end of the treatment manipulators 400 inserted into the first channel tube 171 protrudes from and retract to the first opening 111a.

In the first opening 111a, a notch portion 111n is formed at both ends in a direction (an LR direction which will be described later) perpendicular to a longitudinal direction A. As illustrated in FIG. 3, the treatment manipulators 400 protruding from the first opening 111a to the distal end side A1 can be inserted into the notch portions 111n. The notch portions 111n may not penetrate in the direction perpendicular to the longitudinal direction A as long as they are formed on the inner circumferential surface of the first opening 111a.

The second opening 111b is an opening that communicates with the second channel tube 172. The second opening 111b is a circular opening in a front view when seen from the distal end side A1. The distal end of the treatment manipulator 400 inserted into the second channel tube 172 protrudes from and retracts to the second opening 111b. Devices inserted into the second channel tube 172 are not limited to the treatment manipulator 400 and include medical devices such as manual instruments.

An inner diameter D1 of a part other than the notch portions 111n in the first opening 111a is larger than an inner diameter D2 of the second opening 111b. Specifically, the diameter D1 of the part other than the notch portions 111n in the first opening 111a is three to five times the inner diameter D2 of the second opening 111b.

The water supply nozzle 111d is an opening that communicates with the water supply tube 175. A liquid in a tank installed in the vicinity of the control device 700 is sent out from the water supply nozzle 111d via the water supply tube 175.

The air supply nozzle 111e is an opening that communicates with the air supply tube 176. A gas in a tank installed in the vicinity of the control device 700 is sent out from the air supply nozzle 111e via the air supply tube 176.

The suction nozzle 111f is an opening that communicates with the suction tube 177. A tank installed in the vicinity of the control device 700 sucks a gas or a liquid from the suction nozzle 111f via the suction tube 177.

### [Scope 200]

The scope 200 is a unit that observes a lesion or the like and is attached to the distal end portion 111. The scope 200 may be bendably attached to protrude from the distal end portion 111 to the distal end side A1. The scope 200 includes an imaging unit 201 and an illumination unit 202.

The imaging unit (camera) 201 includes a stereo lens and an imaging element such as a CMOS and images an imaging subject. An imaging signal is sent to the video control device 600 via the imaging cable 173.

The illumination unit 202 is connected to a light guide 174 that guides illumination light and emits illumination light illuminating an imaging subject.

In the electric endoscope system 1000, the entire insertion manipulator 100 may be considered to be an “endoscope.” In addition, the scope 200, the imaging cable 173, and the light guide 174 may be considered to be an “endoscope.”

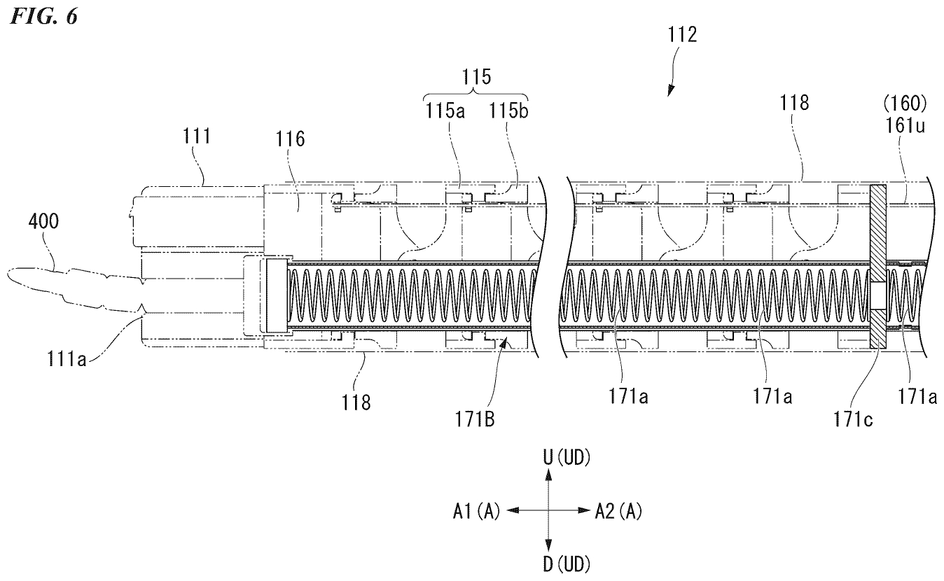

FIG. 6 is diagram illustrating the bending portion 112.

The bending portion 112 includes a plurality of joint rings (also referred to as bending pieces) 115, distal end portions 116 connected to the distal ends of the plurality of joint rings 115, and an outer sheath 118. The plurality of joint rings 115 are connected in the longitudinal direction A in the outer sheath 118. The joint ring 115 at the distal end is connected to the distal end portion 111. In the bending portion 112 illustrated in FIG. 3, the outer sheath 118 is not illustrated.

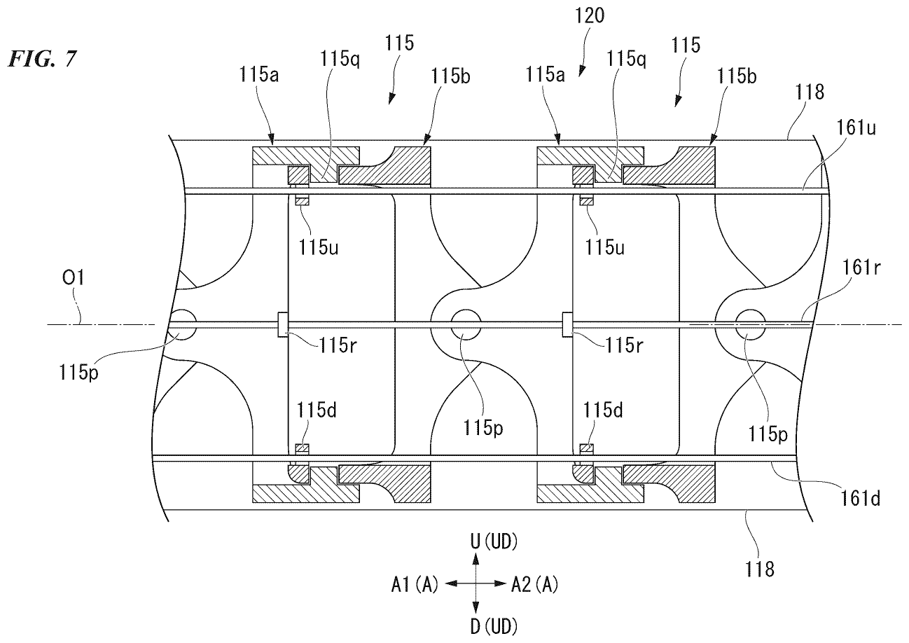

FIG. 7 is a diagram illustrating joint rings 115.

A joint ring 115 is a short cylindrical member formed of metal. A plurality of joint rings 115 are connected such that internal spaces of the neighboring joint rings 115 form a continuous space.

Each joint ring 115 includes a first joint ring 115a on the distal end side and a second joint ring 115b on the proximal side. The first joint ring 115a and the second joint ring 115b are connected by a first rotary pin 115p to be rotatable in a vertical direction (also referred to as an “UD direction”) perpendicular to the longitudinal direction A.

In the neighboring joint rings 115, the second joint ring 115b of the joint ring 115 on the distal end side and the first joint ring 115a of the joint ring 115 on the proximal side are connected by a second rotary pin 115q to be rotatable in a lateral direction (also referred to as an “LR direction”) perpendicular to the longitudinal direction A and the UD direction.

The first joint rings 115a and the second joint rings 115b are alternately connected by the first rotary pin 115p and the second rotary pin 115q, and the bending portion 112 is bendable in a desired direction.

An upper wire-guide 115u and a lower wire-guide 115d are formed on the inner circumferential surface of the second joint ring 115b. The upper wire-guide 115u and the lower wire-guide 115d are disposed at both ends in the UD direction with the center axis O1 in the longitudinal direction A interposed therebetween. A left wire-guide 1151 and a right wire-guide 115r are formed in the inner circumferential surface of the first joint ring 115a. The left wire-guide 1151 and the right wire-guide 115r are disposed at both ends in the LR direction with the center axis O1 in the longitudinal direction A interposed therebetween.

The upper wire-guide 115u, the lower wire-guide 115d, the left wire-guide 1151, and the right wire-guide 115r are formed such that through-holes into which the bending wire 160 is inserted are parallel to the longitudinal direction A.

The bending wire 160 is a wire for bending the bending portion 112. The bending wire 160 extends to the attachment portion 150 via the internal path 101. The bending wire 160 includes an upper bending wire 161u, a lower bending wire 161d, a left bending wire 1611 (see FIG. 3), and a right bending wire 161r.

The upper bending wire 161u and the lower bending wire 161d are wires for bending the bending portion 112 in the UD direction. The upper bending wire 161u is inserted into the upper wire-guide 115u. The lower bending wire 161d is inserted into the lower wire-guide 115d.

The left bending wire 1611 and the right bending wire 161r are wires for bending the bending portion 112 in the LR direction. The left bending wire 1611 is inserted into the left wire-guide 1151. The right bending wire 161r is inserted into the right wire-guide 115r.

The distal end of the bending wire 160 is fixed to the distal end portion 116 of the bending portion 112. The bending portion 112 is bendable in a desired direction by pulling or relaxing the bending wires 160 (the upper bending wire 161u, the lower bending wire 161d, the left bending wire 1611, and the right bending wire 161r).

The bending wire 160 and the built-in elements 170 (the first channel tube 171, the second channel tube 172, the imaging cable 173, the light guide 174, the water supply tube 175, the air supply tube 176, and the suction tube 177) are inserted into the internal path 101 formed in the bending portion 112. In FIG. 6, built-in elements 170 other than the first channel tube 171 are not illustrated.

The flexible portion 119 is a tubular member which is longitudinal and flexible. The flexible portion 119 includes an outer sheath 118 which is the outermost part. The distal end of the flexible portion 119 is connected to the bending portion 112. The bending wire 160 and the built-in elements 170 (the first channel tube 171, the second channel tube 172, the imaging cable 173, the light guide 174, the water supply tube 175, the air supply tube 176, and the suction tube 177) are inserted into the internal path 101 formed in the flexible portion 119.

The attachment portion 150 is provided at the proximal end of the flexible portion 119 as illustrated in FIG. 1. The attachment portion 150 is attached to the drive device 500 and the video control device 600.

### [First Channel Tube 171]

As illustrated in FIG. 5, the first channel tube 171 is a tube including a first treatment tool lumen 171r with a large diameter. The second channel tube 172 is a tube including a second treatment tool lumen 172r. The inner diameter D1 of the first treatment tool lumen 171r is larger than the inner diameter D2 of the second treatment tool lumen 172r. Specifically, the inner diameter D1 of the first treatment tool lumen 171r is three to five time the inner diameter D2 of the second treatment tool lumen 172r. As illustrated in FIG. 3, two treatment manipulators 400 can be inserted into the first treatment tool lumen 171r.

The inner diameter D12 of the first treatment tool lumen 171r is equal to or greater than half the outer diameter of the outer sheath 118. In an existing endoscope (for example, a nasal endoscope) in which the inner diameter of a treatment tool channel is relatively large, the outer diameter is about 6 mm and the inner diameter of the treatment tool channel is about 2.4 mm (about ⅖ times the outer diameter). In the insertion manipulator 100, the inner diameter D1 of the first treatment tool lumen 171r with respect to the outer diameter of the outer sheath 118 is larger than that in the existing endoscope. Accordingly, a large treatment manipulator 400 can be inserted into the first treatment tool lumen 171r, and a range of a manual operation is widened.

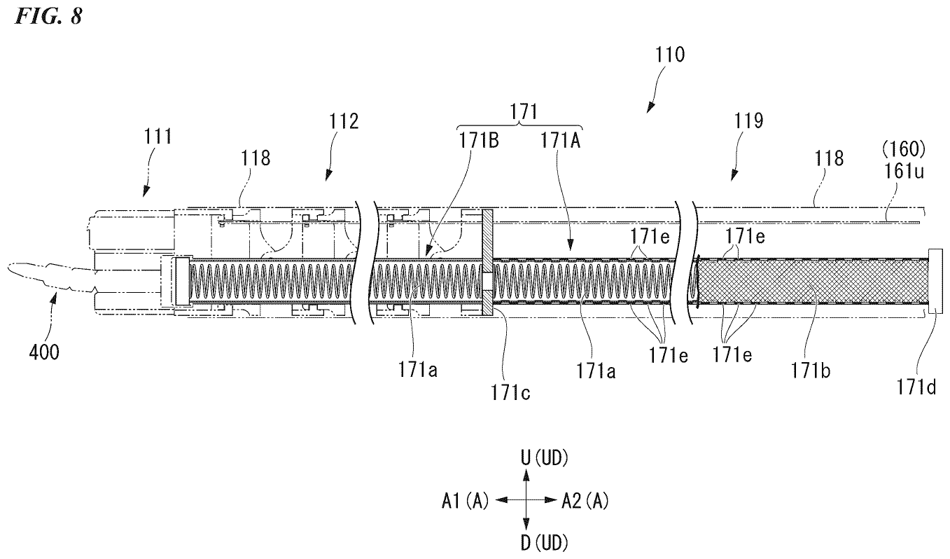

FIG. 8 is a diagram illustrating the first channel tube 171. In FIG. 8, the built-in elements 170 other than the first channel tube 171 are not illustrated.

The first channel tube 171 includes a proximal end channel tube 171A disposed in the flexible portion 119 and a distal end channel tube 171B disposed in the bending portion 112. The proximal end channel tube 171A and the distal end channel tube 171B are connected to form the first treatment tool lumen 171r.

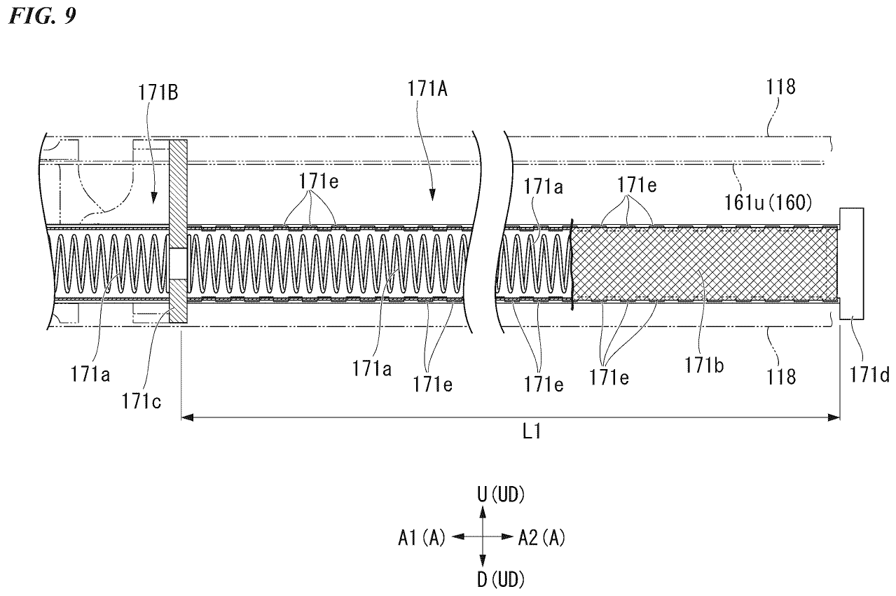

FIG. 9 is a sectional view of the proximal end channel tube 171A.

The proximal end channel tube 171A includes a coil sheath 171a, a blade tube 171b, a first fixed portion 171c, a second fixed portion 171d, and a regulating wire 171e.

The coil sheath 171a is a flexibly bendable coil sheath having excellent kink-resistance. The coil sheath 171a forms the first treatment tool lumen 171r with a large diameter into which the treatment manipulator 400 is inserted.

The blade tube 171b is a tube in which metal strands, resin strands, or the like are woven in a blade shape and is disposed outside of the coil sheath 171a. It is preferable that the blade tube 171b be disposed to be in contact with an outer circumferential surface of the coil sheath 171a.

The proximal end channel tube 171A is a tube with a double structure of the coil sheath 171a and the blade tube 171b. Accordingly, the proximal end channel tube 171A also has excellent torque transmissivity based on the blade tube 171b while maintaining excellent kink-resistance based on the coil sheath 171a. By fitting the coil sheath 171a and the blade tube 171b, a frictional force between the coil sheath 171a and the blade tube 171b is further enhanced, and the torque transmissivity is further enhanced.

The coil sheath 171a is fixed to the blade tube 171b at a first fixed portion 171c on the distal end side A1 in the axial direction A and a second fixed portion 171d on the proximal side A2 with respect to the first fixed portion 171c.

The regulating wire 171e is connected to the first fixed portion 171c and the second fixed portion 171d and regulates a distance L1 in the axial direction A between the first fixed portion 171c and the second fixed portion 171d. It is preferable that a plurality of regulating wires 171e be provided. The regulating wire 171e is a wire with high rigidity and is, for example, an NiTi wire.

Since the distance L1 in the axial direction A between the first fixed portion 171c and the second fixed portion 171d is regulated by the regulating wire 171e, it is possible to prevent a decrease in torque transmissivity or a decrease in pushability due to expansion and contraction of the coil sheath 171a and the blade tube 171b.

The regulating wire 171e is provided to move alternately inside and outside of the blade tube 171b. Accordingly, the regulating wire 171e can appropriately regulate the distance L1 in the axial direction A between the first fixed portion 171c and the second fixed portion 171d regardless of a bent shape of the insertion section 110.

The coil sheath 171a may be fixed to the blade tube 171b by the first fixed portion 171c and the second fixed portion 171d in a state in which the coil sheath is compressed in the axial direction A. In this case, the coil sheath 171a has a biasing force for separating the first fixed portion 171c and the second fixed portion 171d in the axial direction A. A tension in the axial direction A acts on the blade tube 171b. As a result, it is possible to prevent a decrease in torque transmissivity or a decrease in pushability due to contraction of the coil sheath 171a and the blade tube 171b. When this effect is not sufficient, the regulating wire 171e is not necessarily required.

As described above, the proximal end channel tube 171A includes the first treatment tool lumen 171r with a large diameter, but has excellent kink-resistance, excellent torque transmissivity, and excellent pushability. Accordingly, even when the outer sheath 118 which is the outermost part of the flexible portion 119 is thin, the flexible portion 119 can maintain excellent kink-resistance, excellent torque transmissivity, and excellent pushability.

The distal end channel tube 171B may have the same configuration as the proximal end channel tube 171A. However, the bending portion 112 in which the distal end channel tube 171B is provided includes the joint rings 115 with high rigidity and thus has excellent torque transmissivity and pushability. Accordingly, the distal end channel tube 171B has only to include the coil sheath 171a forming the first treatment tool lumen 171r and may not include the blade tube 171b and the regulating wire 171e.

Since the outer sheath 118 can be formed of a thin film as described above, it is possible to achieve a decrease in diameter of the insertion manipulator 100. However, when the first channel tube 171 does not have sufficient kink-resistance, torque transmissivity, or pushability, the outer sheath 118 may be a multi-layered tube including an outer coil sheath into which the first channel tube 171 is inserted and an outer blade tube disposed outside of the outer coil sheath.

### [Drive Device 500]

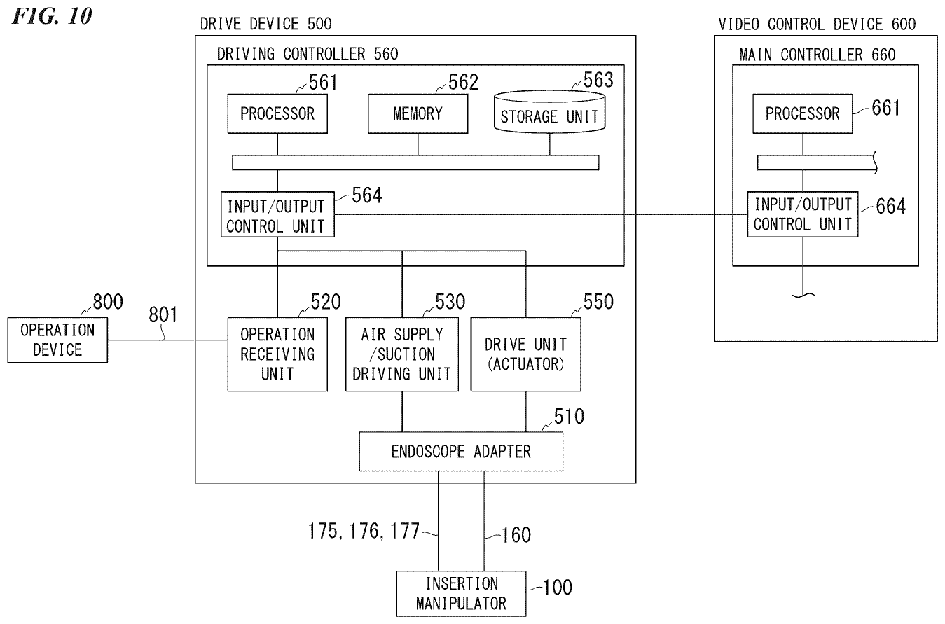

FIG. 10 is a functional block diagram illustrating the drive device 500.

The drive device 500 includes an endoscope adapter 510, an operation-receiving unit 520, an air supply/suction driving unit 530, a drive unit 550, and a drive controller 560.

The endoscope adapter 510 is an adapter to which the insertion manipulator 100 is detachably connected. The endoscope adapter 510 connects the bending wire 160, the suction tube 177, the water supply tube 175, and the air supply tube 176 to the drive device 500.

The operation-receiving unit 520 receives an operation input from the operation device 800 via the operation cable 801. When the operation device 800 and the drive device 500 communicate in a wireless manner instead of a wired manner, the operation-receiving unit 520 includes a known wireless receiver module.

The air supply/suction driving unit 530 is connected to the suction tube 177, the water supply tube 175, and the air supply tube 176 via the endoscope adapter 510. The air supply/suction driving unit 530 includes a pump and supplies a liquid to the water supply tube 175. The air supply/suction driving unit 530 supplies air to the air supply tube 176. The air supply/suction driving unit 530 sucks air from the suction tube 177.

The drive unit (actuator) 550 is connected to the bending wire 160 of the insertion manipulator 100 via the endoscope adapter 510. The drive unit 550 includes a drive unit and an encoder which are not illustrated. The drive unit pulls or relaxes the bending wire 160 using a pulley or the like. The encoder detects an amount of pulling of the bending wire 160. The detection result of the encoder is acquired by the drive controller 560 of the drive device 500.

The drive unit (actuator) 550 may be connected to the treatment manipulator 400 to drive the treatment manipulator 400.

The drive controller 560 controls the drive device 500 as a whole. The drive controller 560 acquires the operation input received by the operation-receiving unit 520. The drive controller 560 controls the air supply/suction driving unit 530 and the drive unit 550 on the basis of the acquired operation input.

The drive controller 560 is a program-executable computer including a processor 561, a memory 562, a storage unit 563 storing a program and data, and an input/output control unit 564. The functions of the drive controller 560 are realized by causing the processor 561 to execute a program. At least some functions of the drive controller 560 may be realized by a dedicated logical circuit.

The drive controller 560 may further include an element in addition to the processor 561, the memory 562, the storage unit 563, and the input/output control unit 564. For example, the drive controller 560 may further include an image computing unit that performs some or all of image processing and image recognition processing. When the image-computing unit is additionally provided, the drive controller 560 can perform specific image processing or image recognition processing at a high speed. The image-computing unit may be mounted in a separate hardware device connected thereto via a communication line.

### [Video Control Device 600]

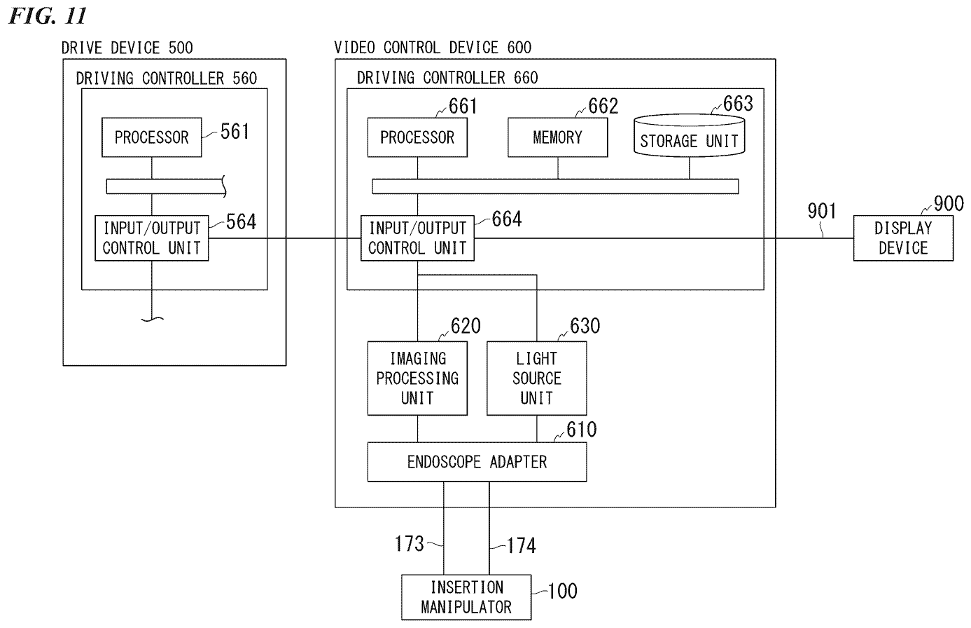

FIG. 11 is a functional block diagram illustrating the video control device 600.

The video control device 600 includes an endoscope adapter 610, an imaging processing unit 620, a light source unit 630, and a main controller 660.

The endoscope adapter 610 is an adapter to which the insertion manipulator 100 is detachably connected. The endoscope adapter 610 connects the imaging cable 173 and the light guide 174 to the video control device 600.

The imaging processing unit 620 is connected to the imaging cable 173. The imaging processing unit 620 converts an imaging signal acquired from the imaging unit 201 of the scope 200 via the imaging cable 173 and the imaging cable 173 to a captured image.

The light source unit 630 is connected to the light guide 174. The light source unit 630 generates illumination light applied to an imaging subject. The illumination light generated by the light source unit 630 is guided to an illumination unit 202 of the scope 200 via the light guide 174.

The main controller 600 is a program-executable computer including a processor 661, a memory 662, a storage unit 663 that can store a program and data, and an input/output control unit 664. The functions of the main controller 660 are realized by causing the processor 661 to execute a program. As least some functions of the main controller 660 may be realized by a dedicated logical circuit.

The main controller 660 can perform image processing on a captured image acquired by the imaging processing unit 620. The main controller 660 can generate a GUI image or a CG image to provide information to an operator S. The main controller 660 can display the captured image, the GUI image, or the CG image on the display device 900.

The main controller 660 is not limited to an integrated hardware device. For example, the main controller 660 may be constituted by separating a part thereof as a separate hardware device and connecting the separated hardware device thereto via a communication line. For example, the main controller 660 may be a cloud system that connects the separated storage units 663 via a communication line.

The main controller 660 may further include an element in addition to the processor 661, the memory 662, the storage unit 663, and the input/output control unit 664. For example, the main controller 660 may further include an image-computing unit that performs some or all of image processing or image recognition processing performed by the processor 661. Since the image-computing unit is further provided, the main controller 660 can perform specific image processing or image recognition processing at a high speed. The image-computing unit may be mounted in a separate hardware device connected thereto via a communication line.

### [Operation of Electric Endoscope System 1000]

Operations of the electric endoscope system 1000 according to the present embodiment will be described below. Specifically, manual operations of observing and treating a lesion formed in a wall of a large intestine using the electric endoscope system 1000 will be described.

An operator S inserts the insertion section 110 of the insertion manipulator 100 into the large intestine via an anal passage of a patient from its distal end. The operator S operates the operation device 800 while observing the captured image displayed on the display device 900 such that the insertion section 110 is moved to cause the distal end portion 111 to approach a lesion. The operator S operates the operation device 800 to bend the bending portion 112 according to necessity.

Since the insertion manipulator 100 has excellent kink-resistance, excellent torque transmissivity, and excellent pushability, the operator S can easily operate the insertion manipulator 100.

The operator S inserts the treatment manipulator 400 into the first channel tube 171 or the second channel tube 172. The operator S operates the operation device 800 while observing the captured image displayed on the display device 900 such that the treatment manipulator 400 is operated to treat the lesion.

Since the insertion manipulator 100 includes the first treatment tool lumen 171r with a large diameter, the operator S can appropriately perform treatment using the large treatment manipulator 400.

After treating the lesion, the operator S removes the insertion manipulator 100 and the treatment manipulator 400 and ends the manual operation.

With the electric endoscope system 1000 according to the present embodiment, it is possible to more efficiently perform observation or treatment. Since the insertion manipulator 100 has excellent kink-resistance, excellent torque transmissivity, and excellent pushability, the operator S can easily operate the insertion manipulator 100. Since the insertion manipulator 100 includes the first treatment tool lumen 171r with a large diameter, the operator S can appropriately perform treatment using the large treatment manipulator 400.

While the first embodiment of the present invention has been described above in detail with reference to the drawings, a specific configuration is not limited to this embodiment and includes changes in design without departing from the gist of the present invention. Elements described in the aforementioned embodiment and modified examples can be appropriately combined into a configuration.

### Modified Example 1-1

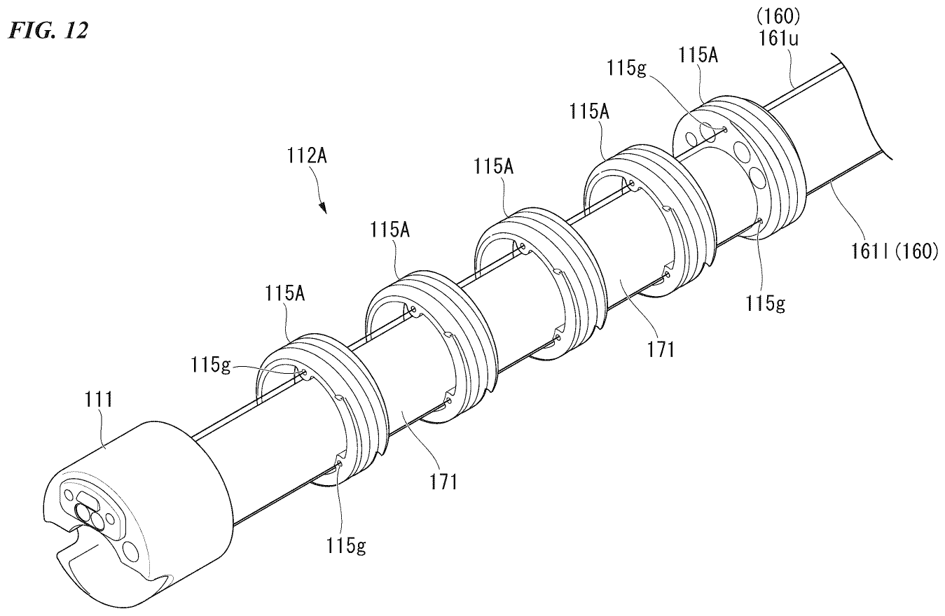

FIG. 12 is a diagram illustrating a bending portion 112A which is a modified example of the bending portion 112. In FIG. 12, the built-in elements 170 other than the first channel tube 171 are not illustrated.

The bending portion 112 does not include a plurality of joint rings 115 but includes a plurality of ring members 115A. The plurality of ring members 115A are arranged in the axial direction A. The built-in elements 170 including the first channel tube 171 are inserted into the plurality of ring members 115A. The bending wire 160 is inserted into a wire-guide 115g formed in the ring members 115A.

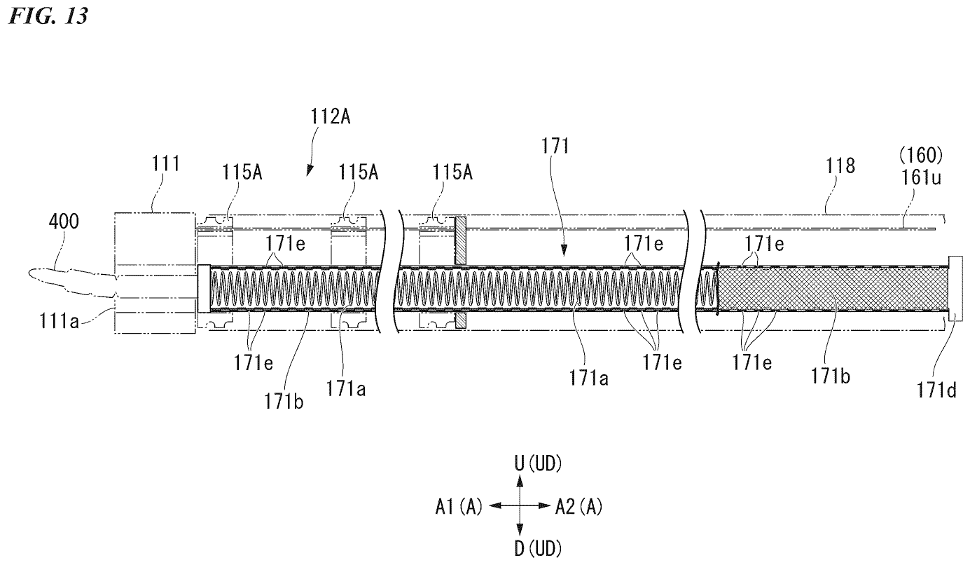

FIG. 13 is a diagram illustrating the first channel tube 171 inserted into the bending portion 112A.

The bending portion 112A has lower torque transmissivity or lower pushability in comparison with the bending portion 112 in which the joint rings 115 are connected. Accordingly, a distal end portion of the first channel tube 171 into which the bending portion 112A is inserted is preferably a tube with a double structure of the coil sheath 171a and the blade tube 171b. The distal end portion of the first channel tube 171 into which the bending portion 112A is inserted preferably includes a regulating wire 171e for regulating a distance between the ends.

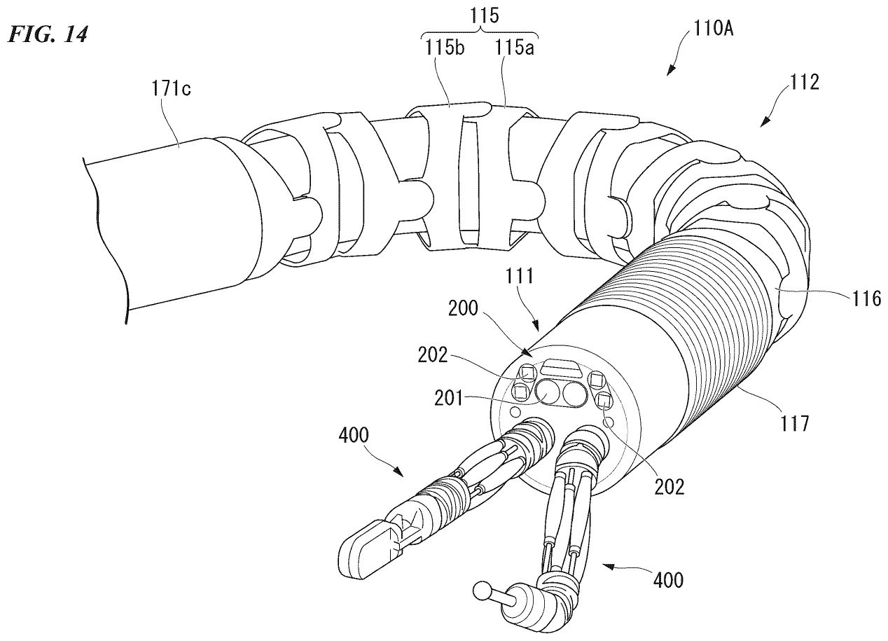

FIG. 14 is a diagram illustrating an insertion section 110A which is a modified example of the insertion section 110.

The insertion section 110A includes an expanding and contracting portion 117 in addition to a distal end portion 111, a bending portion 112, and a flexible portion 119.

The expanding and contracting portion 117 is a member that connects the distal end portion 116 of the bending portion 112 to the distal end portion 111 and is driven by the drive unit (actuator) 550 to expand and contract in the longitudinal direction A. An outer circumferential portion of the expanding and contracting portion 117 is formed in a bellows shape. The operator S can accurately control a position of the distal end portion 111 in the longitudinal direction A with expansion and contraction of the expanding and contracting portion 117.

FIG. 15 is a diagram illustrating a spiral tube 180.

The operator may insert the insertion section 110 into a spiral tube 180 which is separate from the insertion section 110. The spiral tube 180 is a tube in which a fin 181 is wound in a spiral shape on an outer circumference thereof. The spiral tube 180 is driven by the drive unit (actuator) 550 to be rotatable about a rotation axis extending in the longitudinal direction A. The operator can cause the spiral tube 180 and the insertion section 110 to move forward and rearward in the lumen by rotating the spiral tube 180. For example, the operator can easily insert the distal end portion 111 of the insertion section 110 to a deep region of a large intestine.

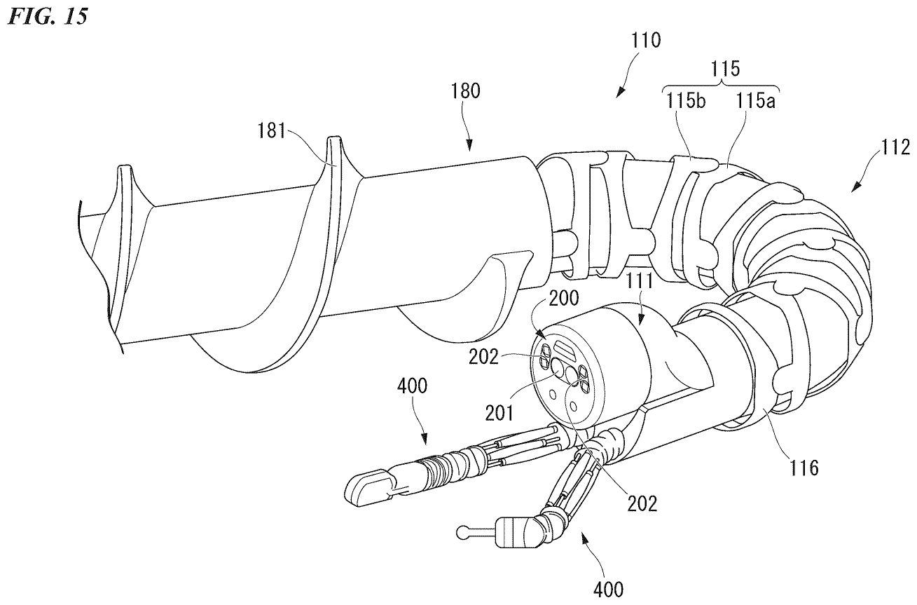

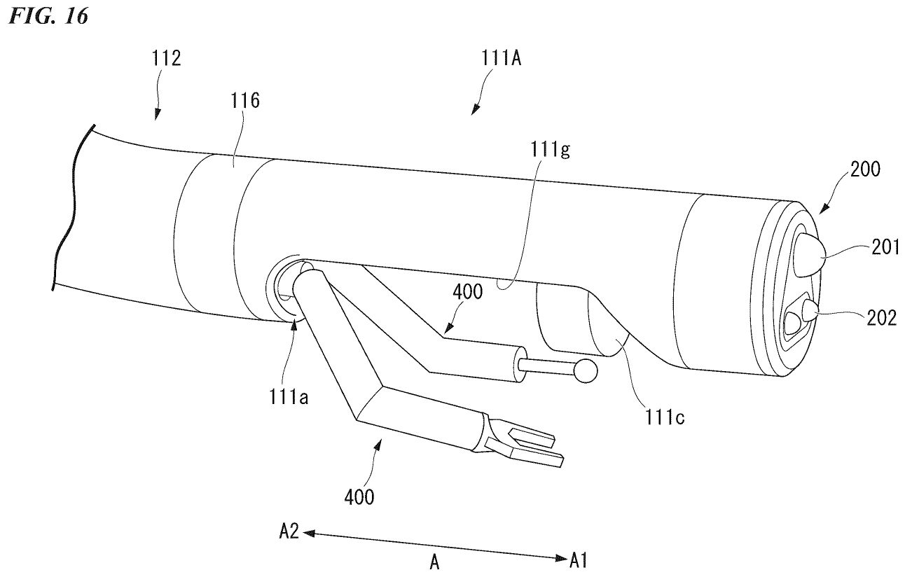

FIG. 16 is a diagram illustrating a distal end portion 111A which is a modified example of the distal end portion 111.

The distal end portion 111A includes a cutout portion 111g in which a part of a cylindrical body is cut out. The cutout portion 111g extends in the longitudinal direction A. A first opening 111a of the distal end portion 111A is provided on the proximal side A2 of the cutout portion 111g. The first opening 111a is open to the distal end side A1. The scope 200 is attached to the distal end of the distal end portion 111A. The distal end portion 111A includes a treatment camera 111c in addition to the scope 200 which is an insertion camera. The treatment camera 111c is provided on the distal end side A1 of the cutout portion 111g. The first opening 111a and the treatment camera 111c are provided to face each other. The treatment camera 111c can image a situation in which the treatment manipulator 400 protruding from the first opening 111a treats a lesion. Since the direction which the treatment camera 111c faces and the direction in which the treatment manipulator 400 protrudes are opposite, the operator can perform treatment while viewing the distal end of the treatment manipulator 400.

### Second Embodiment

A hardness-changing device 300 according to a second embodiment of the present disclosure will be described below with reference to FIGS. 17 to 25. In the following description, the same constituents as in the above description will be referred to by the same reference signs, and repeated description thereof will be omitted.

FIG. 17 is a diagram of a hardness-changing device 300 inserted into the bending portion 112.

The hardness-changing device (rigidizer, introducer) 300 is a device including a hardness-changing portion 310 into which the first treatment tool lumen 171r can be inserted. In FIG. 17, the hardness-changing device 300 is inserted into the first treatment tool lumen 171r. In FIG. 17, the first channel tube 171 and the second channel tube 172 are not illustrated.

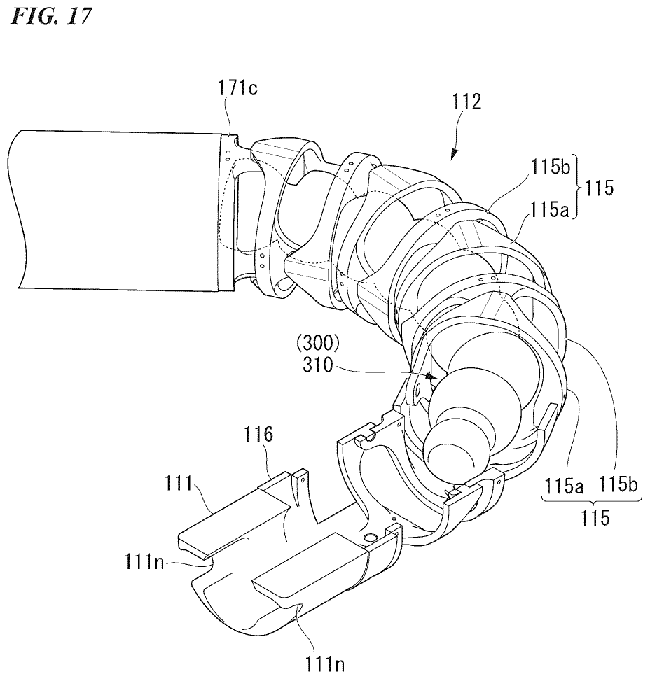

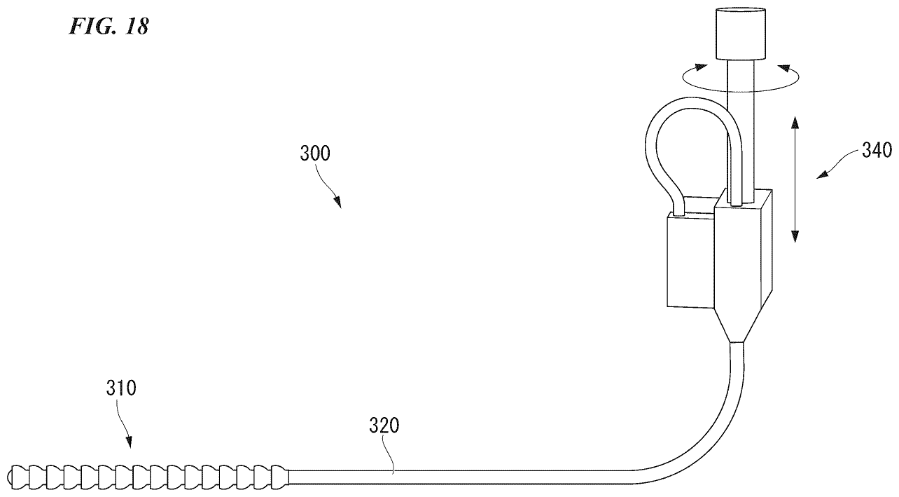

FIG. 18 is a diagram illustrating the entire configuration of the hardness-changing device 300.

The hardness-changing device 300 includes a hardness-changing portion 310, an insertion section 320, and a drive unit 340.

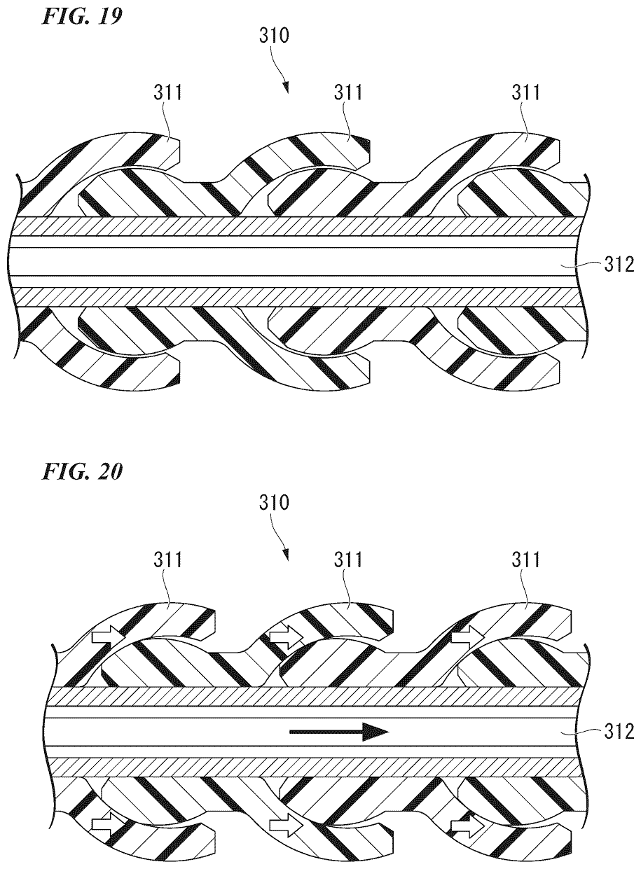

FIGS. 19 and 20 are diagrams illustrating the hardness-changing portion 310.

The hardness-changing portion 310 is a long member which can be inserted into the first treatment tool lumen 171r and in which hardness changes by performing a predetermined operation thereon. The hardness-changing portion 310 includes a plurality of spinal portions 311 and a wire 312. Each spinal portion 311 is formed in a bowl shape. The plurality of spinal portions 311 are connected in a longitudinal axis direction while overlapping each other. The wire 312 is inserted into the plurality of spinal portions 311 and is fixed to a spinal portion at the distal end. When the wire 312 is pulled to the proximal side as illustrated in FIG. 20, neighboring spinal portions 311 come into close contact, and thus a frictional force is enhanced and the shape of the hardness-changing portion 310 is fixed. By loosening the wire 312, the shape of the hardness-changing portion 310 changes.

The insertion section 320 is a long member that can be inserted into the first treatment tool lumen 171r and has flexibility. The insertion section 320 is provided on the proximal side of the hardness-changing portion 310.

The drive unit 340 is provided on the proximal side of the insertion section 320. The drive unit 340 causes the insertion section 320 to move forward and rearward in the longitudinal axis direction and causes the insertion section 320 to rotate about the longitudinal axis. The drive unit 340 may be incorporated into the drive device 500.

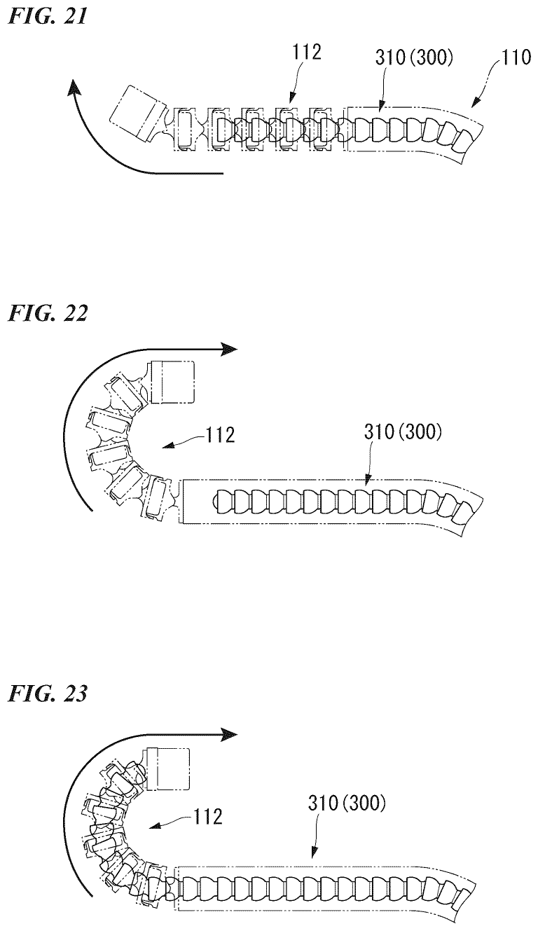

FIGS. 21 to 25 are diagrams illustrating operations of the hardness-changing portion 310.

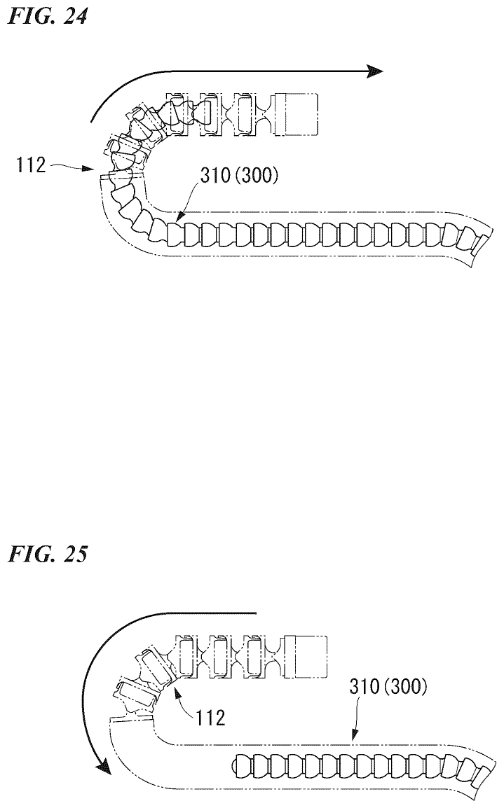

As illustrated in FIG. 21, the operator S inserts the distal end of the insertion section 110 of the insertion manipulator 100 into a large intestine of a patient via an anal passage. As illustrated in FIG. 22, the operator S disposes the bending portion 112 in a part of the large intestine which is greatly bent. As illustrated in FIG. 23, the operator S causes the hardness-changing portion 310 with a variable shape to move forward and locates the hardness-changing portion 310 in the bending portion 112. The operator S fixes the shape of the hardness-changing portion 310 inserted into the bending portion 112. As illustrated in FIG. 24, the operator S causes the insertion section 110 to move forward. The bending portion 112 moves forward along the hardness-changing portion 310 with a fixed shape. The insertion manipulator 100 can smoothly pass through the part of the large intestine which is greatly bent. When the hardness-changing portion 310 is not necessary, the operator S opens the wire of the hardness-changing portion 310 and removes the hardness-changing portion 310 from the bending portion 112 in a state in which it is softened as illustrated in FIG. 25.

With the hardness-changing device 300 according to the present embodiment, it is possible to more efficiently perform observation or treatment. The operator S can smoothly insert the insertion manipulator 100 to a target position using the hardness-changing device 300.

While the second embodiment of the present invention has been described above in detail with reference to the drawings, a specific configuration is not limited to this embodiment and includes changes in design without departing from the gist of the present invention. Elements described in the aforementioned embodiment and modified examples can be appropriately combined into a configuration.

### Modified Example 2-1

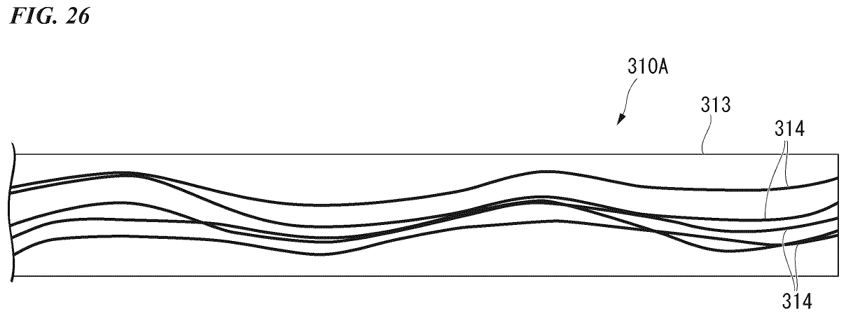

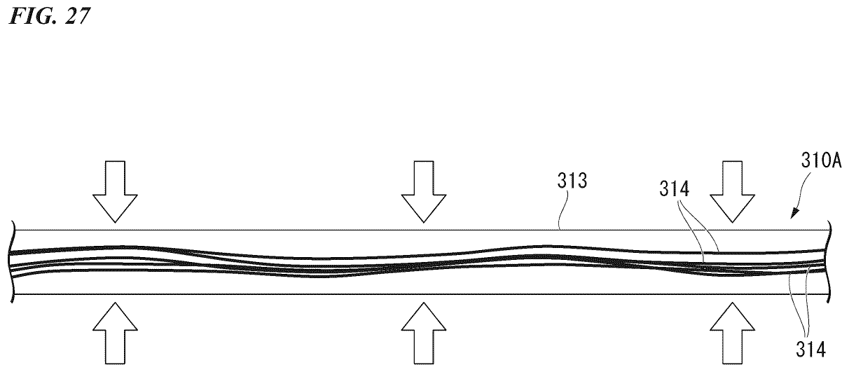

FIGS. 26 and 27 are diagrams illustrating a hardness-changing portion 310A which is a modified example of the hardness-changing portion 310. The hardness-changing portion 310A includes a tube 313 and a plurality of wire members 314 which are inserted into the tube 313. Each wire member 314 is a metal strand or a resin strand. As illustrated in FIG. 27, by making the internal space of the tube 313 have a negative pressure through suction and brining the plurality of wire members 314 into contact with each other, relative movement of the wire members 314 is curbed due to a frictional force between the plurality of wire members 314 and between the wire member 314 and the tube. Accordingly, the shape of the hardness-changing portion 310A is fixed. Since the frictional force is increased by roughening the surfaces of the wire members 314 to increase contact resistance, it is possible to further enhance hardness of the hardness-changing portion 310A with a fixed shape.

### Modified Example 2-2

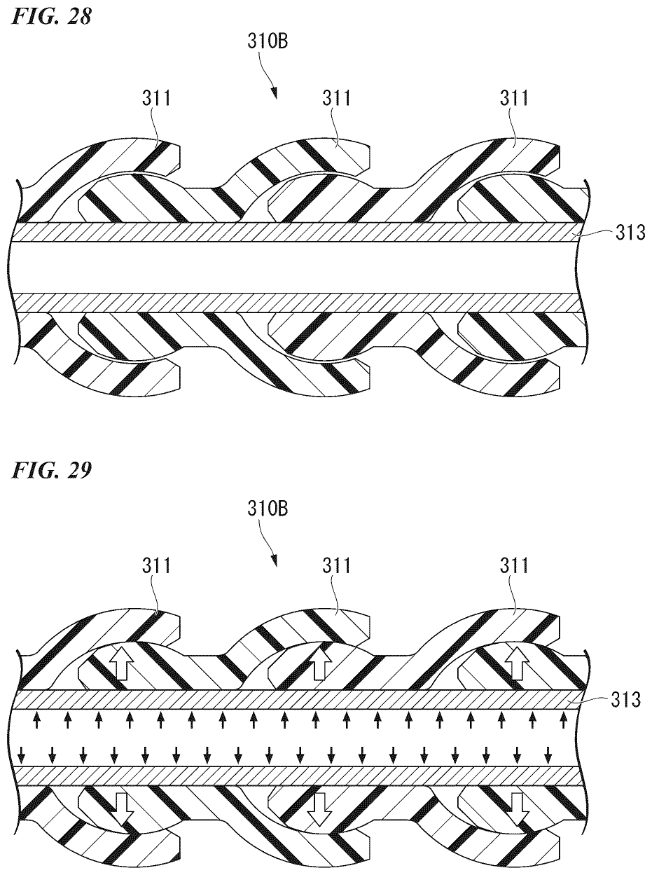

FIGS. 28 and 29 are diagrams illustrating a hardness-changing portion 310B which is a modified example of the hardness-changing portion 310. The hardness-changing portion 310B includes a plurality of spinal portions 311 and a tube 313. The tube 313 is inserted into the plurality of spinal portions 311. As illustrated in FIG. 29, by making the internal space of the tube 313 have a positive pressure through suction such that the tube 313 expands, a frictional force between the neighboring spinal portions 311 is increased, and the shape of the hardness-changing portion 310B is fixed. Since the hardness-changing portion 310B makes the internal space of the tube 313 have a positive pressure, it is possible to easily increase the frictional force between the plurality of spinal portions 311 and to more strength the hardness-changing portion 310B in comparison with a mode in which the internal space of the tube 313 is made to have a negative pressure. In the mode in which the internal space of the tube 313 is made to have a negative pressure, the pressure has only to be reduced by only the atmospheric pressure. However, when the internal space of the tube 313 is made to have a positive pressure, such limitation is removed, and thus it is possible to apply a larger pressure.

### Modified Example 2-3

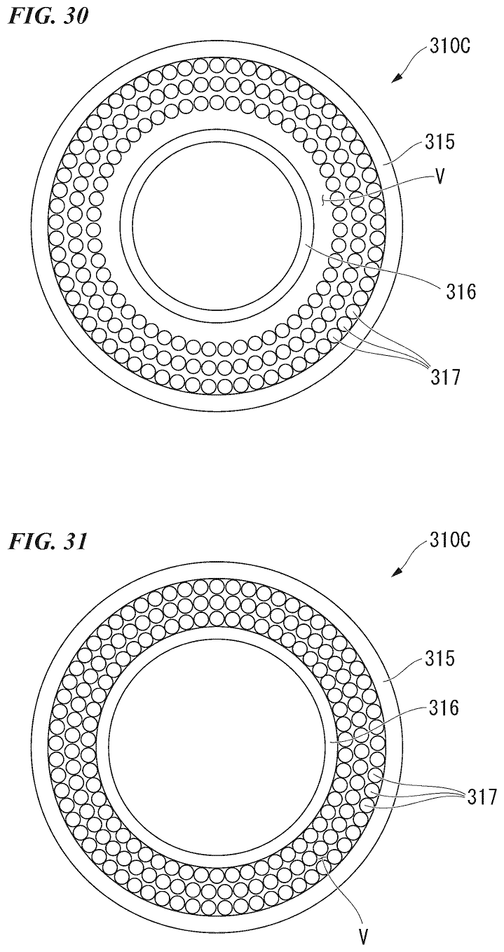

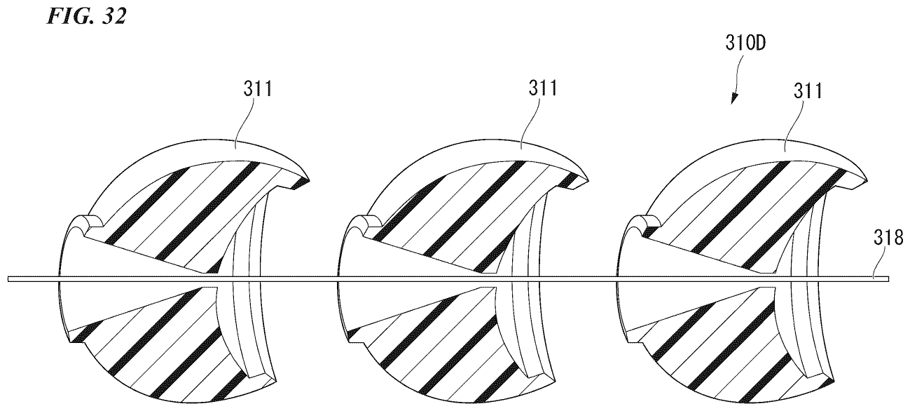

FIGS. 30 and 31 are diagrams illustrating a hardness-changing portion 310C which is a modified example of the hardness-changing portion 310. The hardness-changing portion 310C includes an outer tube 315, an inner tube 316, and a plurality of cables 317. The inner tube 316 is inserted into the inner space of the outer tube 315. The plurality of cables 317 are inserted into a space V interposed between the outer tube 315 and the inner tube 316. As illustrated in FIG. 31, by making the space V have a negative pressure through suction, the plurality of cables 317 come into contact with each other, and the shape of the hardness-changing portion 310C is fixed. The space V may be made to have a negative pressure using a hydraulic pressure.

### Modified Example 2-4

FIG. 32 is a diagram illustrating a hardness-changing portion 310D which is a modified example of the hardness-changing portion 310. The hardness-changing portion 310D includes a plurality of spinal portions 311 and a shape memory alloy wire 318. In the hardness-changing portion 310D, when the shape memory alloy wire 318 contracts with supply of electric power, the spinal portions 311 come into close contact with each other similarly to the hardness-changing portion 310, and thus the hardness-changing portion 310D can switch between a state in which the shape is fixed and a state in which the shape is variable.

### Third Embodiment

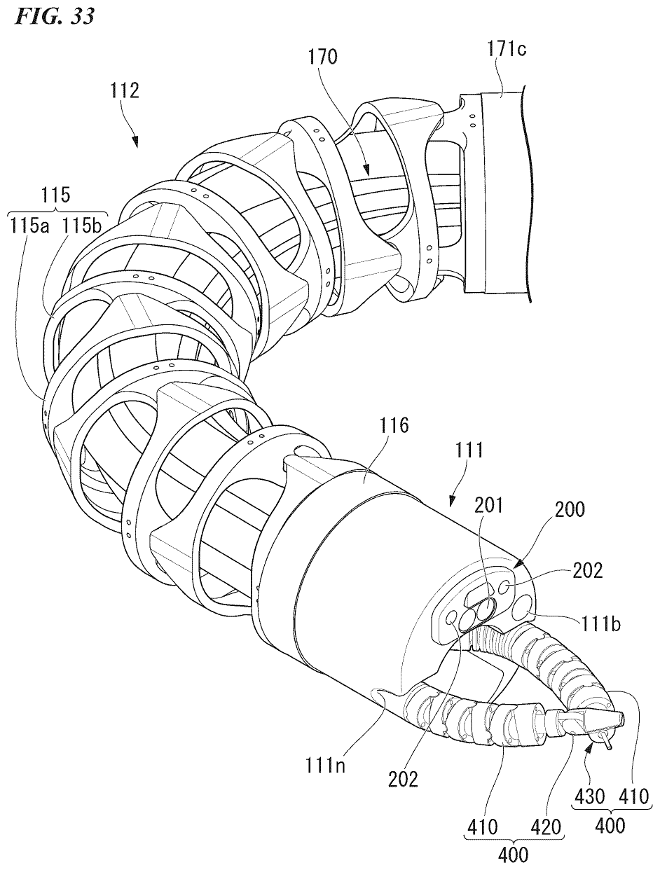

A treatment manipulator 400 according to a third embodiment of the present disclosure will be described below with reference to FIGS. 33 to 40. In the following description, the same constituents as in the above description will be referred to by the same reference signs, and repeated description thereof will be omitted.

FIG. 33 is a diagram illustrating a high-frequency knife 430.

The treatment manipulator 400 includes a bendable treatment tool arm 410 and treatment tools (a forceps 420, a high-frequency knife 430, a local injection needle 440, and a basket 450) which are inserted into the treatment tool arm 410.

The treatment tool arm 410 is a hollow long member. Similar to the bending portion 112 of the insertion manipulator 100, the treatment tool arm 410 includes a plurality of joint rings (also referred to as bending pieces) 415 and is driven with a wire or the like to be bendable in the vertical direction and the lateral direction.

The treatment tools (such as the forceps 420, the high-frequency knife 430, the local injection needle 440, and the basket 450) can be inserted into the internal space (a lumen or a channel) of the treatment tool arm 410. The treatment tools are manually bendable, but may not have an active bending function.

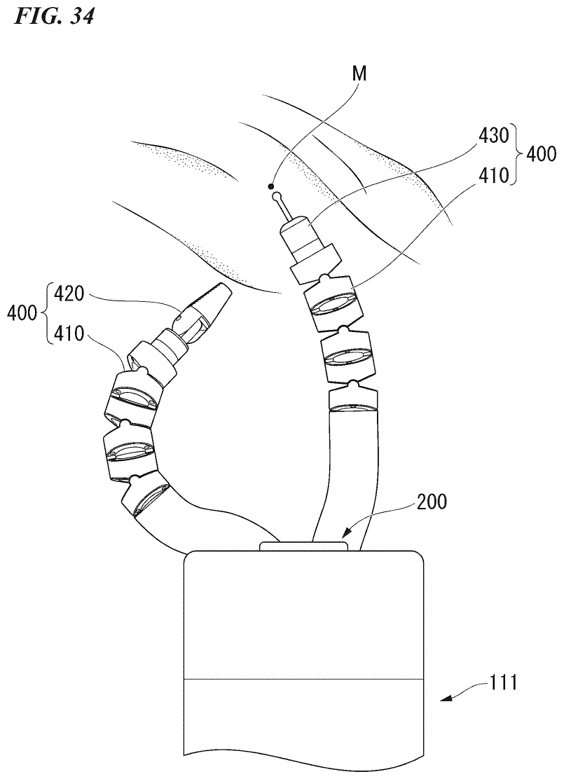

FIG. 34 is a diagram illustrating the high-frequency knife 430 for putting a marking M.

An operator S inserts the high-frequency knife 430 into the treatment tool arm 410 and causes the distal end of the high-frequency knife 430 to protrude from the distal end of the treatment tool arm 410. The operator S locates the distal end of the high-frequency knife 430 at a desired position by bending the treatment tool arm 410. The operator S cauterize biological tissue with the distal end portion of the high-frequency knife 430 and puts a marking M on the biological tissue.

Since the first treatment tool lumen 171r has a large diameter, two treatment tool arms 410 can be inserted thereinto. For example, as illustrated in FIG. 34, the operator S can insert the high-frequency knife 430 into one treatment tool arm 410, insert the forceps 420 into the other treatment tool arm 410, and treat a target part using two treatment tools.

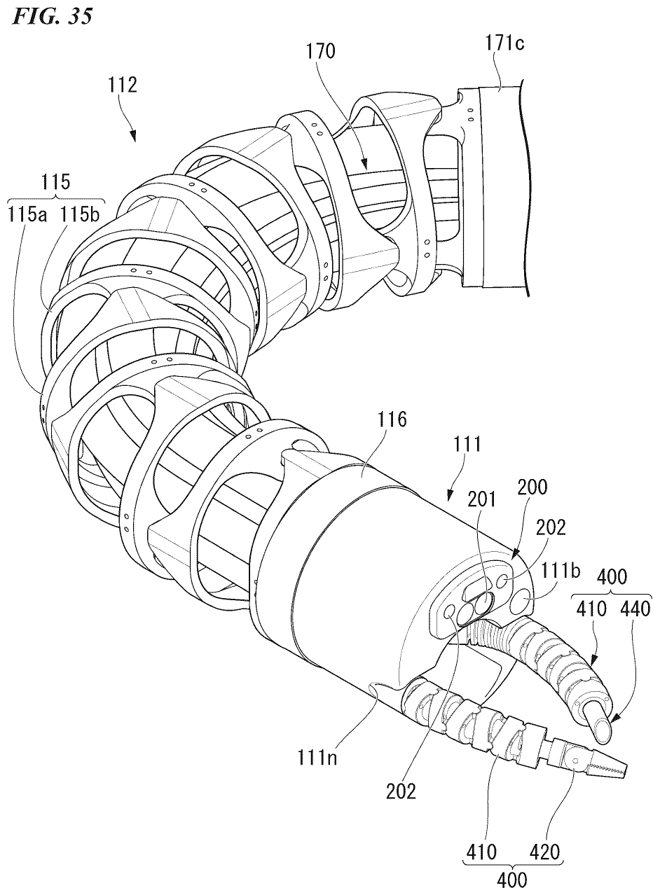

FIG. 35 is a diagram illustrating a local injection needle 440.

The operator S removes the high-frequency knife 430 from the treatment tool arm 410. Then, the operator S inserts the local injection needle 440 into the treatment tool arm 410 and causes the distal end of the local injection needle 440 to protrude from the distal end of the treatment tool arm 410.

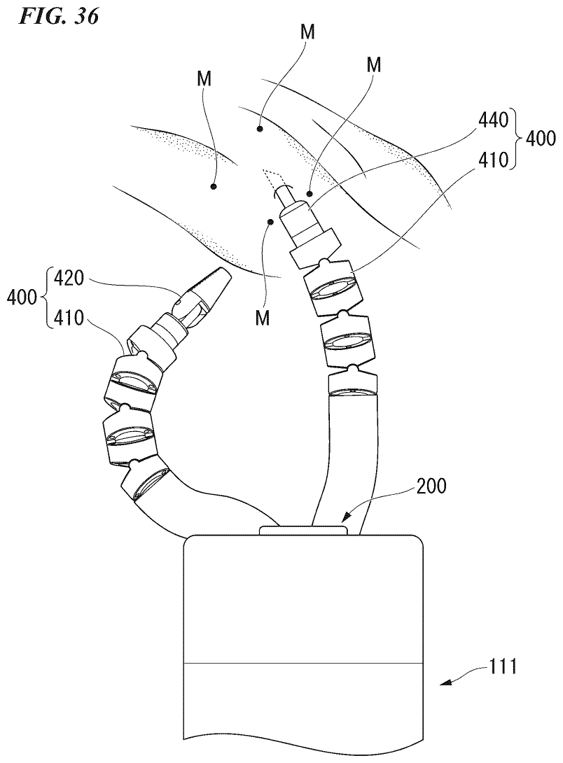

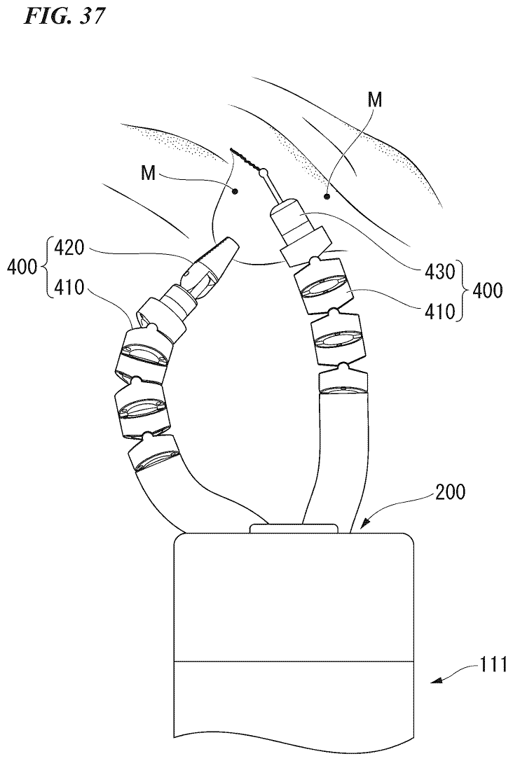

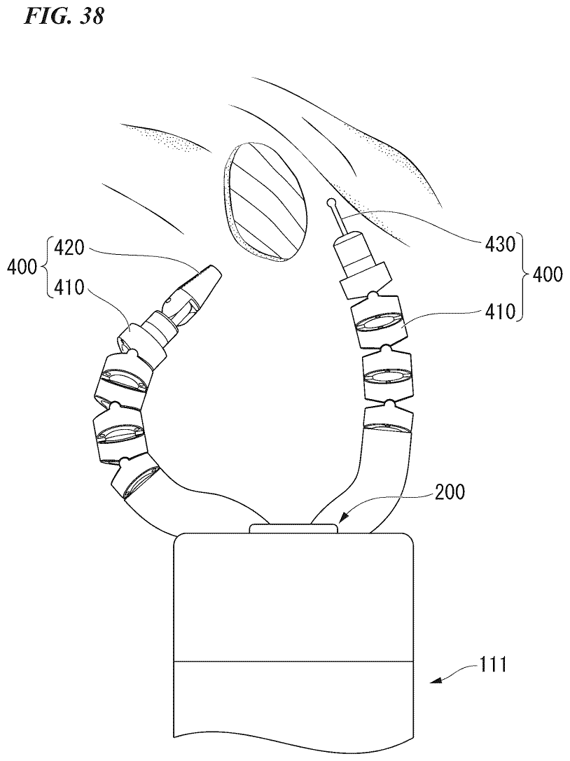

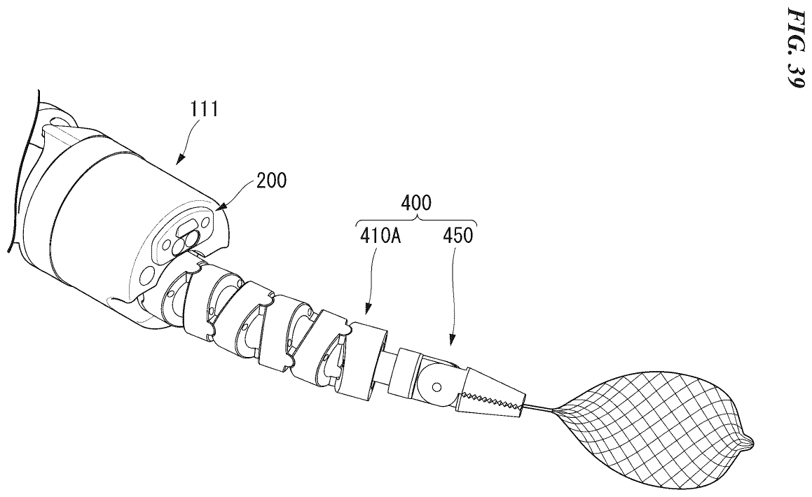

FIG. 36 is a diagram illustrating the local injection needle 440 which locally injects a liquid.

The operator S bends the treatment tool arm 410 and locates the distal end of the local injection needle 440 at a desired position. The operator S pierces the biological tissue with the distal end of the local injection needle 440 and locally injects a liquid.

FIGS. 37 and 38 are diagrams illustrating a high-frequency knife 430 for cutting. The operator S removes the local injection needle 440 from the treatment tool arm 410. Then, the operator S inserts the high-frequency knife 430 into the treatment tool arm 410. The operator S pulls biological tissue using the forceps 420. The operator S cuts a target part of the biological tissue with the high-frequency knife 430 while checking the marking M.

FIG. 39 is a diagram illustrating a basket 450.

The operator S removes the two treatment tool arms 410 along with the treatment tools. Then, the operator S inserts a large-diameter treatment tool arm 410A into the first treatment tool lumen 171r. The operator S inserts the basket 450 into the large-diameter treatment tool arm 410A. The basket 450 has a larger outer diameter than that of the high-frequency knife 430 or the local injection needle 440. The large-diameter treatment tool arm 410A has a larger inner diameter of a hollow portion than that of the treatment tool arm 410 and enables the basket 450 to be inserted thereinto. Only a large basket 450 may be directly inserted into the first treatment tool lumen 171r without using the large-diameter treatment tool arm 410A.

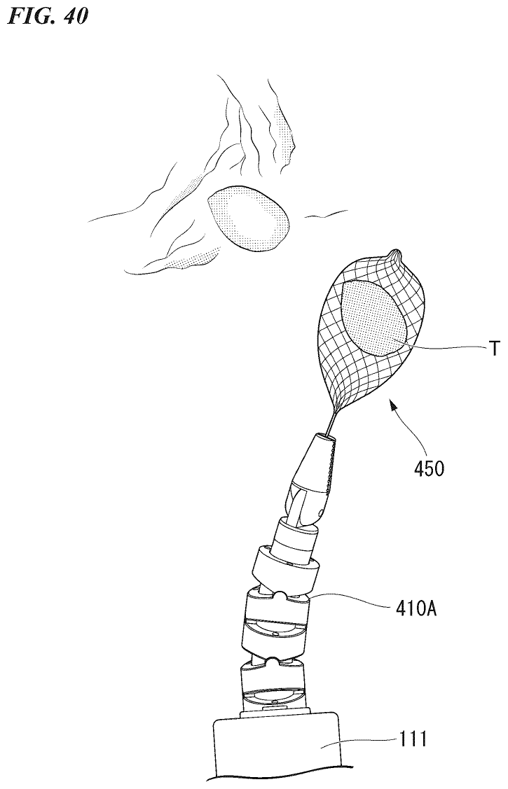

FIG. 40 is a diagram illustrating the basket 450 for recovering a target part T. The operator S recovers a target part T which has been cut and removed using the basket 450. The operator S can efficiently recover the target part T using the large basket 450. Since the first treatment tool lumen 171r has a large diameter, the large basket 450 larger than a basket used in the related art can be inserted thereinto. Since the first treatment tool lumen 171r has a large diameter, a larger amount of target part T or tissue than that in the related art can be easily recovered at a time without being caught by the first treatment tool lumen 171r. Since the first treatment tool lumen 171r has a large diameter, larger treatment tools than those in the related art in addition to the basket can be inserted thereinto.

With the treatment manipulator 400 according to the present embodiment, it is possible to more efficiently perform observation or treatment. Since the insertion manipulator 100 has the first treatment tool lumen 171r with a large diameter, two treatment manipulators 400 can be simultaneously inserted and used. Since the treatment manipulator 400 with a large diameter can be inserted into the first treatment tool lumen 171r, a multi-functional and high-performance large treatment manipulator 400 can be used.

While the third embodiment of the present invention has been described above in detail with reference to the drawings, a specific configuration is not limited to this embodiment and includes changes in design without departing from the gist of the present invention. Elements described in the aforementioned embodiment and modified examples can be appropriately combined into a configuration.

### Fourth Embodiment

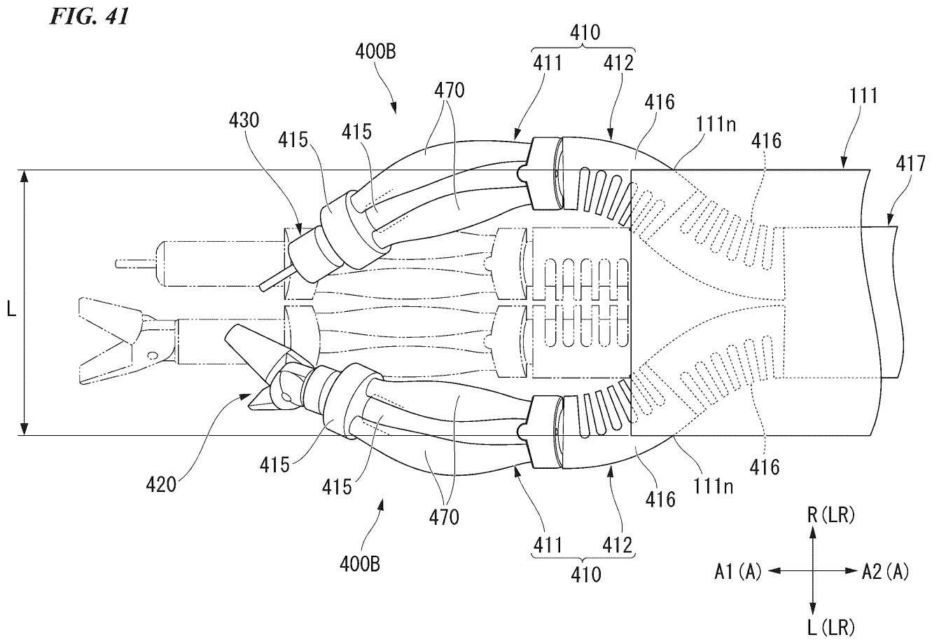

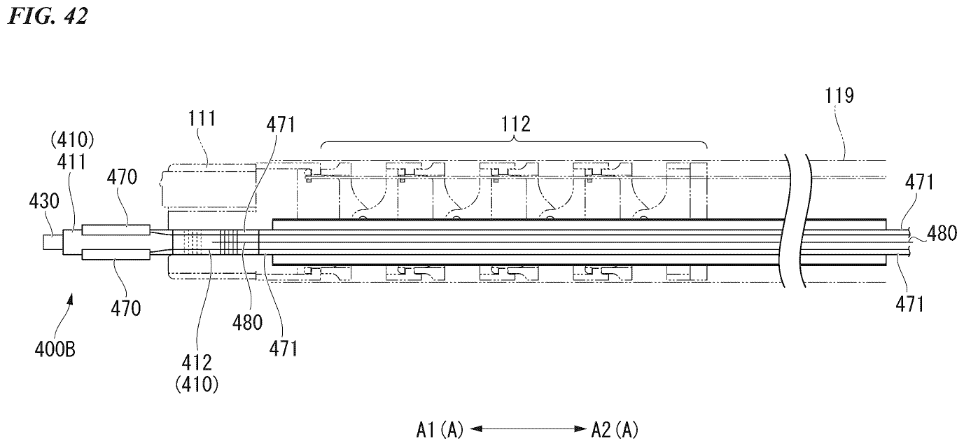

A treatment manipulator 400B according to a fourth embodiment of the present disclosure will be described below with reference to FIGS. 41 to 42. In the following description, the same constituents as in the above description will be referred to by the same reference signs, and repeated description thereof will be omitted.

FIG. 41 is a diagram illustrating a treatment manipulator 400B.

Similar to the treatment manipulator 400 according to the aforementioned embodiment, the treatment manipulator 400B is a device inserted into the first channel tube 171 or the like of the insertion manipulator 100 and is inserted into a lumen of a patient to treat a lesion. The treatment manipulator 400B is driven by the drive unit (actuator) 550 or the like. The treatment manipulator 400B is different from the treatment manipulator 400 in that two treatment tool arms 410 are arranged on a distal end surface of one manipulator flexible portion 417. The treatment manipulator 400B can be more easily inserted in comparison with the treatment manipulator 400.

The treatment manipulator 400B includes a treatment tool (such as a forceps 420, a high-frequency knife 430, a local injection needle 440, or a basket 450), a treatment tool arm 410, an artificial muscle 470, and a wire 480.

The treatment tool arm 410 is a hollow long member. The treatment tool arm 410 includes a first bending portion 411 provided on the distal end side A1 and a second bending portion 412 provided on the proximal side A2. In the usage state of the treatment manipulator 400B illustrated in FIG. 41, the first bending portion 411 is located on the distal end side A1 with respect to the distal end surface of the distal end portion 111. A part of the second bending portion 412 protrudes from the distal end portion 111 to the distal end side A1.

The first bending portion 411 includes a plurality of joint rings (also referred to as wrist joints) 415 similarly to the bending portion 112 of the insertion manipulator 100 and can be driven by the artificial muscle 470 such that it is bendable in the vertical direction and the lateral direction.

As illustrated in FIG. 41, the second bending portion 412 includes a shoulder joint 416 that greatly bends the treatment tool arm 410 in the lateral direction (LR direction). The shoulder joint 416 bends the second bending portion 412 in an S-shape when seen in the vertical direction (UD direction). As illustrated in FIG. 41, the distal ends of the second bending portions 412 of two treatment tool arms 410 are bent and separated from each other in the lateral direction. The distance between the center axes at the distal ends of the second bending portions 412 of the two treatment tool arms 410 is defined as a “distance (shoulder width) L.”

When a target part is treated using two treatment tool arms 410, the operator S can secure sufficient space for treatment and easily treat the target part by appropriately separating the positions of the proximal ends of the two first bending portions 411. Therefore, the operator S greatly bends the second bending portions 412 such that the distance Lis appropriately increased. At least a part of the two treatment manipulators 400 can be inserted into notch portions 111n and spread outward in the lateral direction. Accordingly, the distance L may be larger than the outer diameter of the distal end portion 111 of the insertion manipulator 100.

The artificial muscle 470 constitutes at least a part of a mechanism for bending a treatment tool. The artificial muscle 470 is, for example, a Mckibben artificial muscle. The artificial muscle 470 is attached to the first bending portion 411 and bends the first bending portion 411. In order to bend the first bending portion 411 in the vertical direction and the lateral direction, a plurality of artificial muscles 470 are attached to the first bending portion 411.

In the usage state of the treatment manipulator 400B illustrated in FIG. 41, the first bending portion 411 is disposed on the distal end side A1 with respect to the distal end surface of the distal end portion 111. Accordingly, the artificial muscle 470 is also disposed on the distal end side A1 with respect to the distal end surface of the distal end portion 111.

FIG. 42 is a sectional view of the treatment manipulator 400B.

A tube 471 for supplying a fluid for operating the artificial muscle 470 is attached to the artificial muscle 470. The artificial muscle 470 is contracted with the fluid supplied from the tube 471.

The wire 480 is attached to the second bending portion 412. By pulling the second bending portion 412 from the proximal side A2, the second bending portion 412 is bent.

The first bending portion 411 is bendably driven by the artificial muscle 470. The artificial muscle 470 can more precisely bend the first bending portion 411 in comparison with the wire 480. Accordingly, the artificial muscle 470 is suitable for bendable driving of the first bending portion 411 in which precise operations for treatment are necessary. The artificial muscle 470 is attached to the first bending portion 411 and directly bends the first bending portion 411. Accordingly, it is possible to accurately bend the first bending portion 411 without being affected by the shape on the proximal side of the first bending portion 411.

The second bending portion 412 is bendably driven by the wire 480. The second bending portion 412 does not require a very accurate operation as long as it can greatly bend the shoulder joint 416. Accordingly, it is possible to bend the second bending portion 412 even using the wire 480. Through driving using the wire 480, it is possible to easily secure a longer stroke in comparison with the artificial muscle 470. Accordingly, the wire 480 is suitable for bendable driving of the second bending portion 412 in which a substantial bending operation is necessary.

With the treatment manipulator 400B according to the present embodiment, it is possible to more efficiently perform observation or treatment. Since the first bending portion 411 bendably driven by the artificial muscle 470 can precisely bend the treatment tool, the operator S can more efficiently perform observation or treatment.

While the fourth embodiment of the present invention has been described above in detail with reference to the drawings, a specific configuration is not limited to this embodiment and includes changes in design without departing from the gist of the present invention. Elements described in the aforementioned embodiment and modified examples can be appropriately combined into a configuration.

### Modified Example 4-1

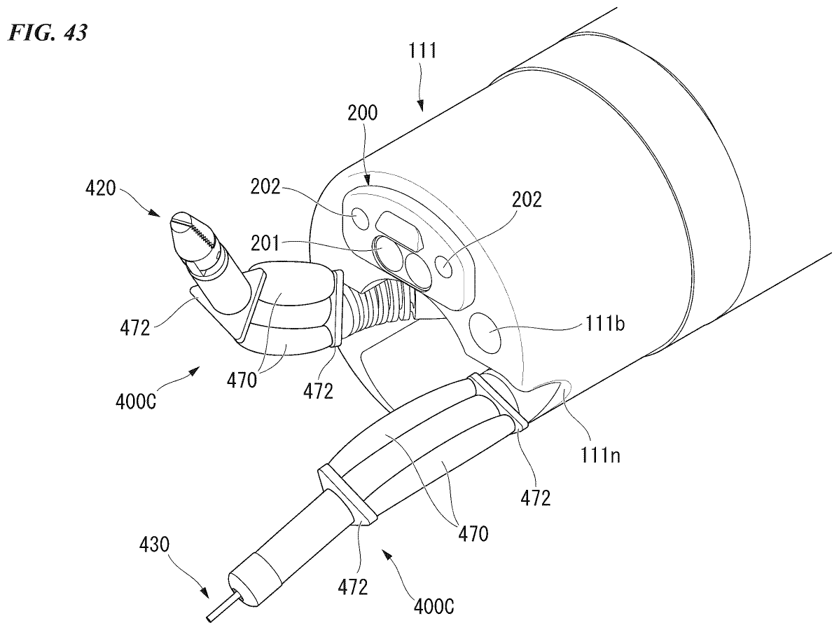

FIG. 43 is a diagram illustrating a treatment manipulator 400C which is a modified example of the treatment manipulator 400B. The treatment manipulator 400C includes a treatment tool and an artificial muscle 470 and does not include a treatment tool arm 410. The artificial muscle 470 is attached to the treatment tool using a ring member 472. A member which is bent with a compression force, for example, a tube or a coil, can be employed instead of the plurality of joint rings 415 illustrated in FIG. 41, and the artificial muscle 470 can be mounted around the member. Instead of a configuration in which the artificial muscle 470 is disposed outside of the joint rings 415, a member which is bent with a compression force, for example, a tube or a coil, may be disposed outside of the artificial muscle 470.

### Modified Example 4-2

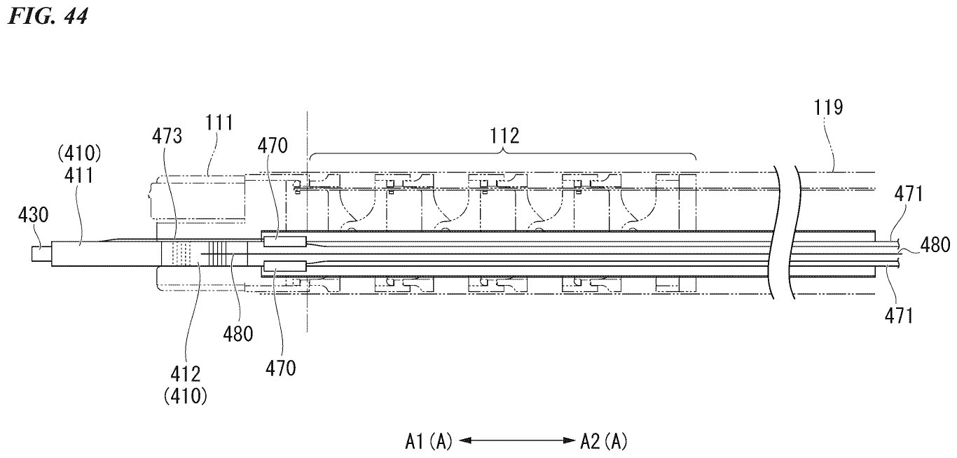

FIG. 44 is a diagram illustrating an artificial muscle 470 disposed at a different position.

The artificial muscle 470 may be provided on the proximal side A2 with respect to the second bending portion 412. The first bending portion 411 and the artificial muscle 470 are connected by a distal end wire 473. The artificial muscle 470 bends the first bending portion 411 by causing the distal end wire 473 to move forward and rearward. The artificial muscle 470 illustrated in FIG. 44 is disposed in the distal end portion 111 on the distal end side A1 with respect to the bending portion 112. The artificial muscle 470 is disposed in the distal end portion 111 which is not bent. Accordingly, even when the bending portion 120 is bent, the artificial muscle 470 is not bent.

### Modified Example 4-3

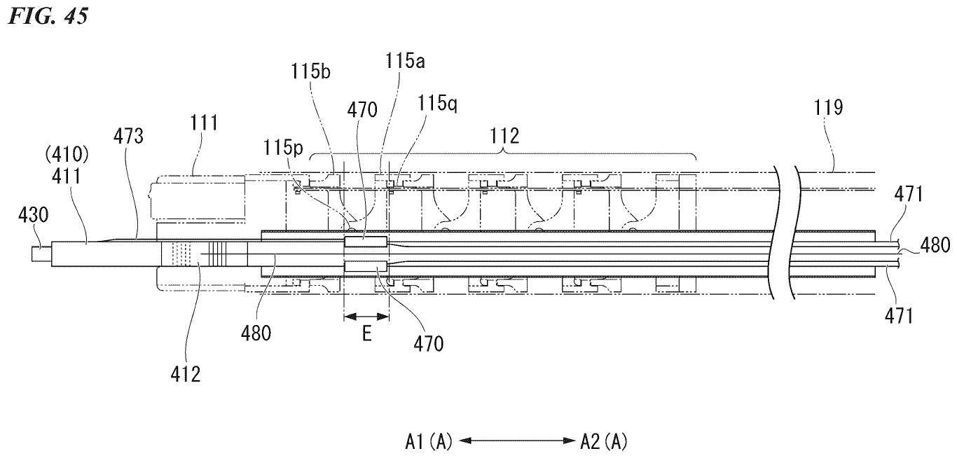

FIG. 45 is a diagram illustrating an artificial muscle 470 disposed at a different position.

The artificial muscle 470 illustrated in FIG. 45 is disposed in the bending portion 112. The artificial muscle 470 is disposed in a non-bending area E which is not bent and which is interposed between a first rotary pin 115p and a second rotary pin 115q. Accordingly, even when the bending portion 120 is bent, the artificial muscle 470 is not bent.

### Modified Example 4-4

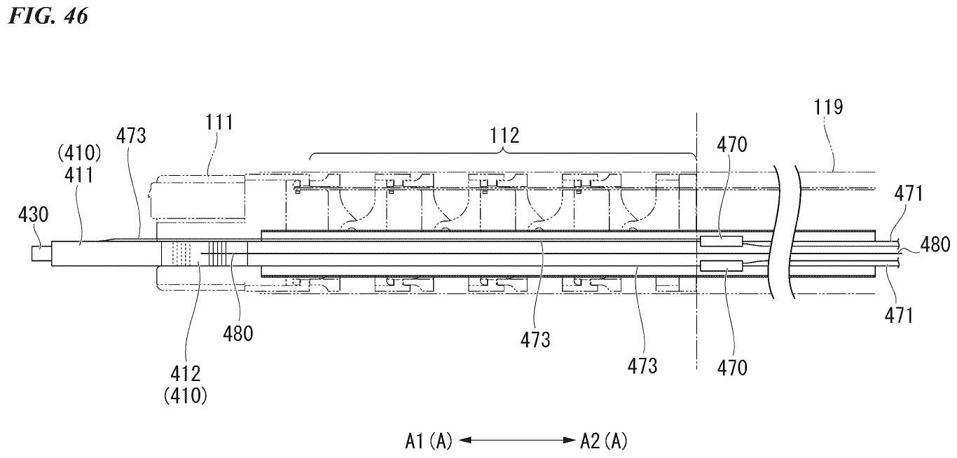

FIG. 46 is a diagram illustrating an artificial muscle 470 which is disposed at a different position.

The artificial muscle 470 illustrated in FIG. 46 is disposed in a flexible portion 119 on the proximal side A2 with respect to the bending portion 112. The artificial muscle 470 is disposed in the flexible portion 119 which is not greatly bent. Accordingly, even when the bending portion 120 is bent, the artificial muscle 470 is not bent.

### Modified Example 4-5

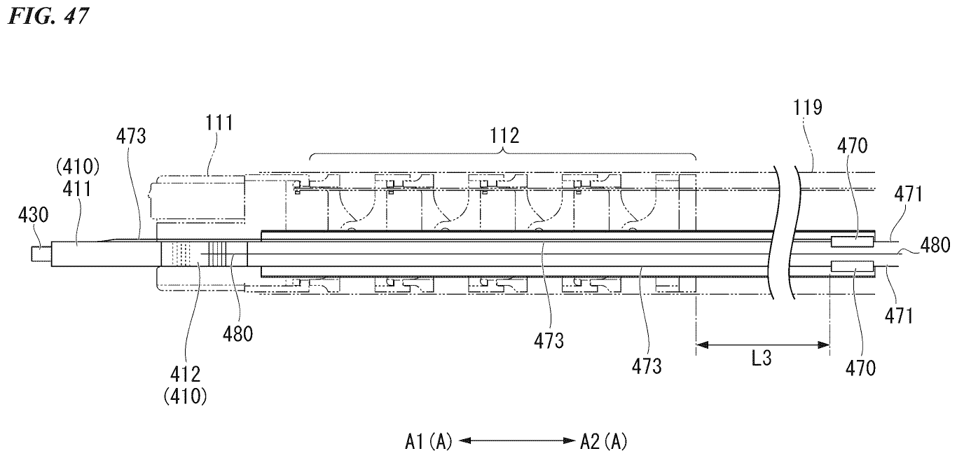

FIG. 47 is a diagram illustrating an artificial muscle 470 which is disposed at a different position.

The artificial muscle 470 illustrated in FIG. 47 is disposed in the flexible portion 119 on the proximal side A2 with respect to the bending portion 112 and is located at a position separated by a distance L3 from the proximal end with respect to the bending portion 112. The distance L3 is longer than a moving distance of the treatment manipulator 400B at the time of treatment. Accordingly, even when the treatment manipulator 400B is intended to move forward and rearward at the time of treatment, the artificial muscle 470 is not inserted into the bending portion 112. Accordingly, even when the bending portion 120 is bent, the artificial muscle 470 is not bent.

### Modified Example 4-6

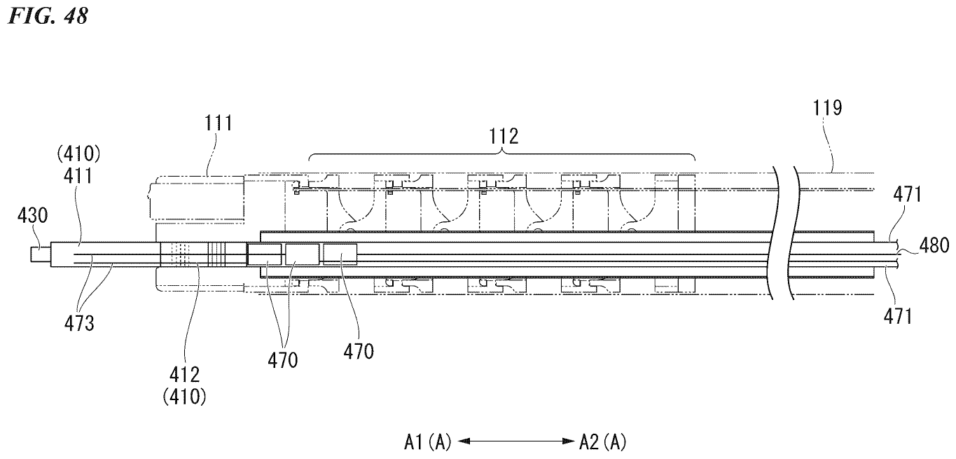

FIG. 48 is a diagram illustrating an artificial muscle 470 which is disposed at a different position.

As illustrated in FIG. 48, a plurality of artificial muscles 470 may be arranged in the axial direction A. It is preferable that some artificial muscles 470 be disposed in a non-bending area. It is not necessary to arrange the plurality of artificial muscles 470 in the radial direction and it is possible to decrease the diameter of the treatment manipulator 400B.

### Modified Example 4-7

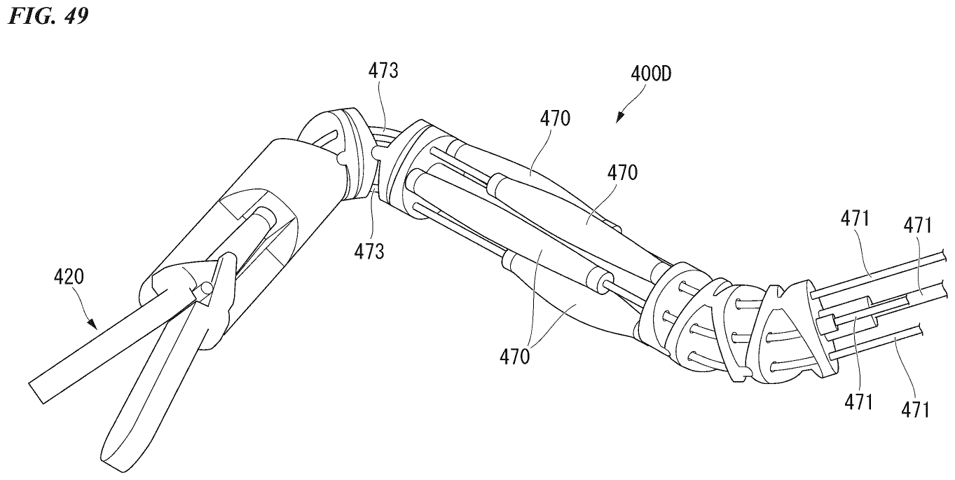

FIG. 49 is a diagram illustrating a treatment manipulator 400D which is a modified example of the treatment manipulator 400B. The treatment manipulator 400D does not include a wire 480 and bends all bending portions using the artificial muscle 470. The operator S can precisely bend all the bending portions.

### Fifth Embodiment

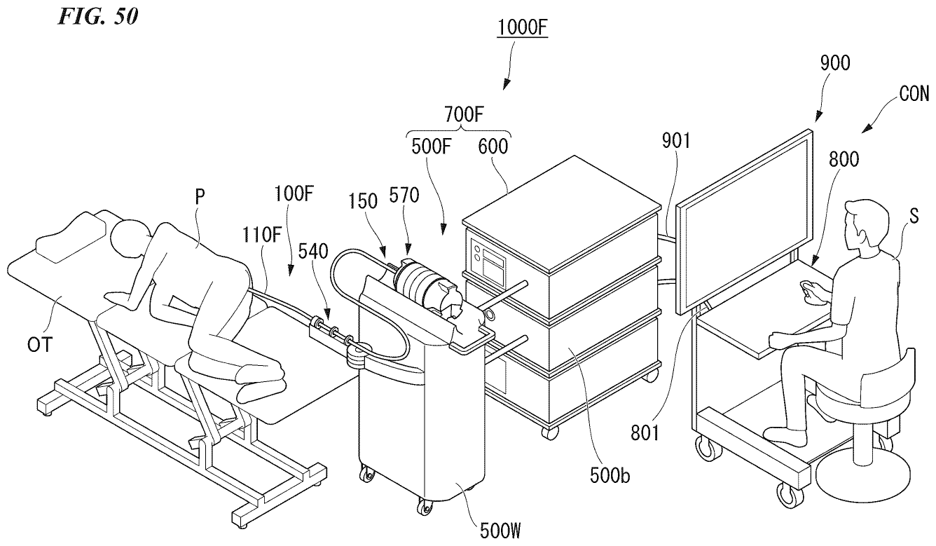

An electric endoscope system 1000F according to a fifth embodiment of the present disclosure will be described below with reference to FIGS. 50 to 60. In the following description, the same constituents as in the above description will be referred to by the same reference signs, and repeated description thereof will be omitted.

FIG. 50 is an overall view of the electric endoscope system 1000F according to this embodiment.

The electric endoscope system 1000F is an example of a medical manipulator system. The medical manipulator includes an insertion manipulator 100F inserted into the body, an electric endoscope, a catheter, a treatment tool, an endoluminal device, and the like.

### [Electric Endoscope System 1000F]

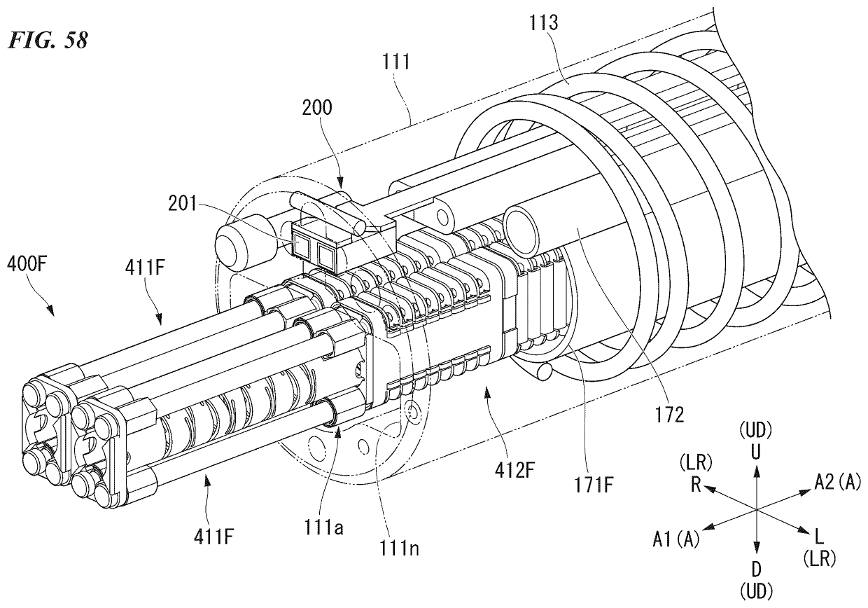

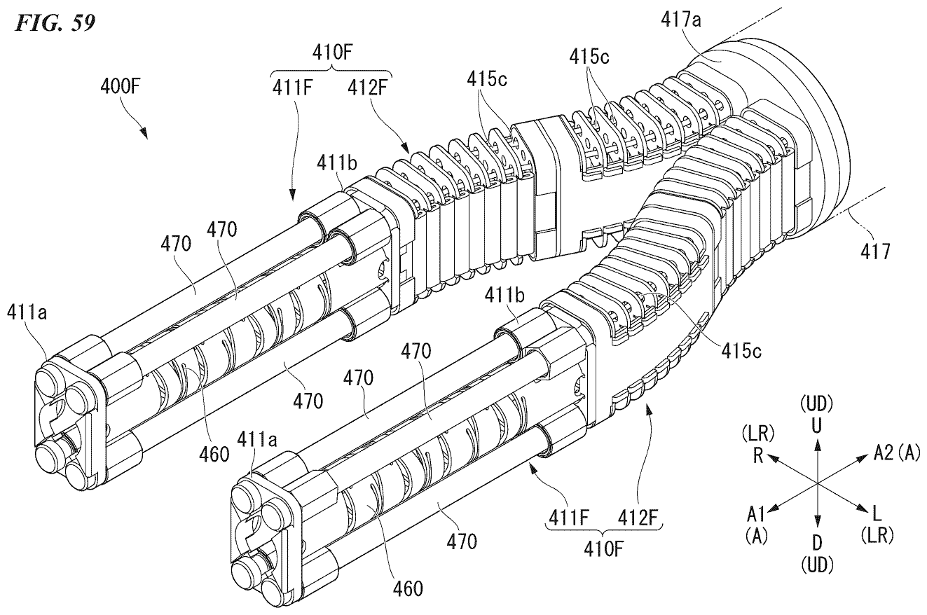

The electric endoscope system 1000F is a medical system for observing and treating the inside of the body of a patient P lying on an operating table OT. The electric endoscope system 1000F includes an insertion manipulator 100F, a treatment manipulator 400F (see FIG. 58), a drive device 500F, an image control device 600, an operation device 800, and a display device 900. The drive device 500F and the image control device 600 constitute a control device 700F that controls the electric endoscope system 1000F.

### [Insertion Manipulator 100F]

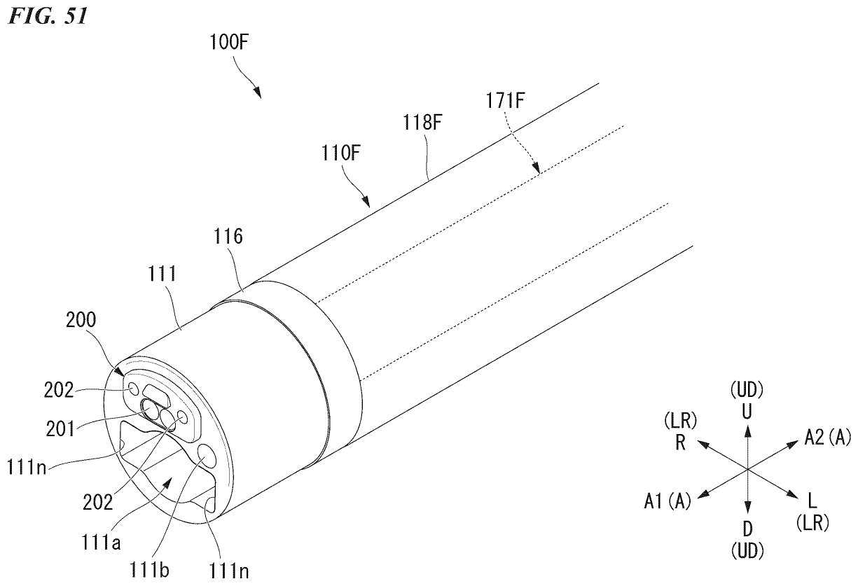

FIG. 51 is a perspective view of the distal end of the insertion manipulator 100F.

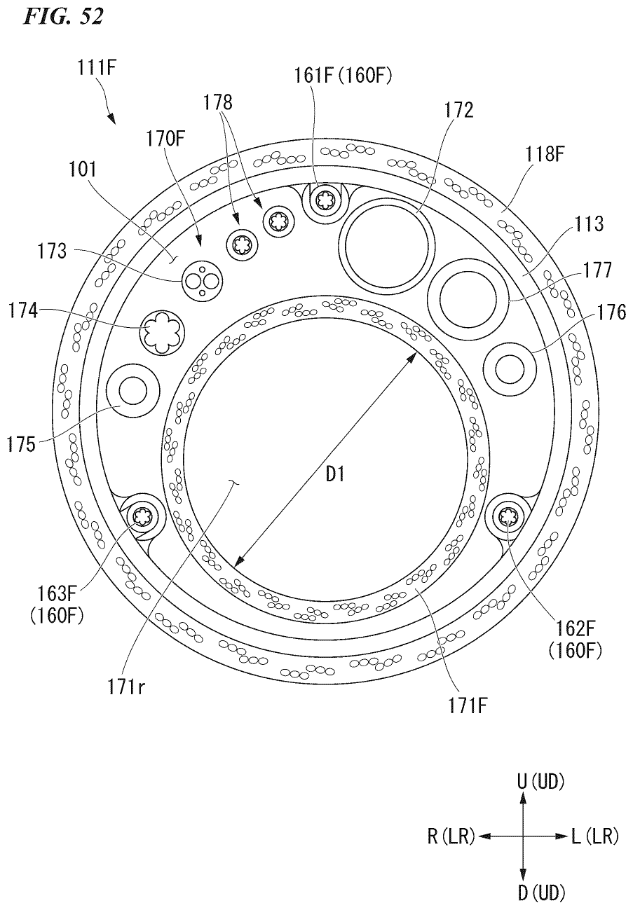

FIG. 52 is a cross-sectional view of the insertion section 110F. The insertion manipulator 100F includes an insertion section 110F, an attachment portion 150, a bending wire 160F, a built-in element 170F, and a scope 200.

Inside the insertion manipulator 100F, an internal passage (lumen) 101 is formed, which extends from the distal end of the insertion section 110F to the proximal end of the attachment portion 150 along the longitudinal direction (longitudinal axis direction, axial direction) A of the insertion manipulator 100F. The bending wire 160F and the built-in element 170F are inserted into the internal passage 101.

The insertion section 110F is a long, thin member that can be inserted into a lumen. The insertion section 110F includes a distal end portion 111, a bending portion 112F, and a flexible portion 119. The distal end portion 111, the bending portion 112F, and the flexible portion 119 are connected in order from the distal end side A1 toward the proximal side A2. The insertion section 110F includes an outer sheath 118F, which is the outermost part.

Similar to the first embodiment, the distal end portion 111 is provided at the distal end of the insertion section 110F. The scope 200 is attached so as to be able to protrude from the distal end portion 111 to the distal end side A1 and bend. The scope 200 is operated by a scope operation wire 178. The notch portion 111n of the distal end portion 111 illustrated in FIG. 51 is formed on the inner circumferential surface of the first opening 111a and does not penetrate in a direction perpendicular to the longitudinal direction A.

As shown in FIG. 52, the built-in element 170F passes through the internal path 101. The built-in element 170 has a first channel tube 171F, a second channel tube 172, an imaging cable 173, a light guide 174, a water supply tube 175, an air supply tube 176, a suction tube 177, and a scope operation wire 178.

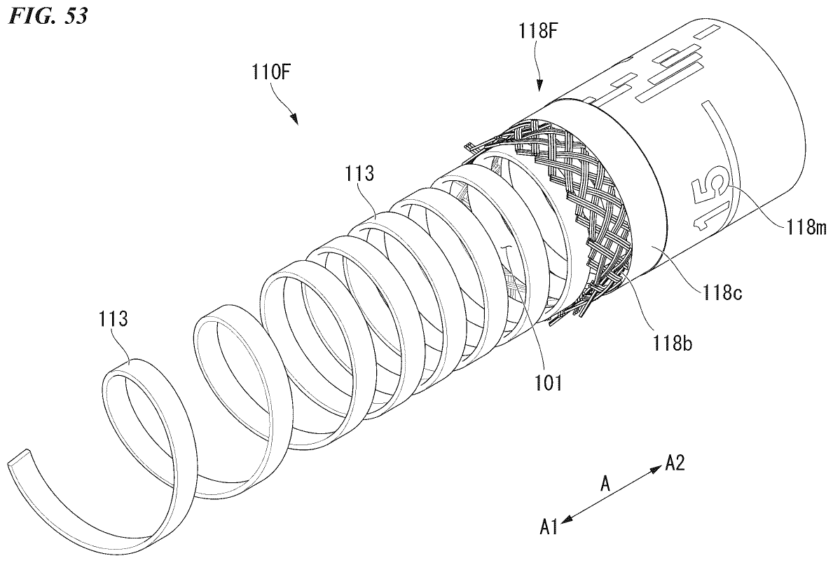

FIG. 53 is a diagram showing the insertion section 110F.

The insertion section 110F includes a spring 113. The spring 113 is formed by winding a linear metal member in a spiral shape. The spring 113 is, for example, a flat wire spring or a round wire spring. By using a flat wire spring instead of a round wire spring formed from a round wire metal member, the diameter of the insertion section 110F is made thinner. On the other hand, by using a round wire spring, it is expected that the insertion section 110F will be easier to bend. In addition, the bending wire 160F and the built-in element 170F are omitted in FIG. 53.

The spring 113 is arranged from the distal end to the proximal end of the insertion section 110F. Therefore, the insertion section 110F has good torque transmission.

The spring 113 is arranged in the internal path 101 of the insertion section 110F in a preloaded state with a compressive force of, for example, 20N to 25N. Since the spring 113 is preloaded, it has good kink resistance and is not easily buckled.

The outer sheath 118F is a cylindrical member arranged on the outside of the spring 113. The outer sheath 118F includes a braided tube 118b and a coating 118c arranged on both the inner and outer sides of the braided tube 118b. The coating 118c arranged on the inner side is also called the inner coating. The coating 118c arranged on the outer periphery side is also called the outer coating. The coating 118c may be either the inner coating or the outer coating.

The braided tube 118b is a tube in which metal wires, resin wires, etc. are woven into a braid shape. The braided tube 118b is made of, for example, aramid or UHMW-PE (Ultra High Molecular Weight Polyethylene).

The coating 118c is sandwiched between the inner and outer periphery sides of the braided tube 118b to seal (encapsulate) the braided tube 118b. The coating 118c is attached to the braided tube 118b by coating, co-extruded, over-casting, or over-molding. The coating 118c is made of, for example, a low-friction, low-durometer resin.

In the outer sheath 118F, the braided tube 118b is sealed (encapsulated) by the coating 118c. Therefore, the braided tube 118b has good rigidity without increasing the diameter. Because the coating 118c has low friction and a low durometer, the outer sheath 118F has suitable elasticity and can balance durability and ease of bending.

The outer surface of the outer sheath 118F is provided with a marking 118m that can be observed under X-ray fluoroscopy. By checking the marking 118m, the surgeon S can easily grasp the insertion position and insertion distance of the insertion section 110F.

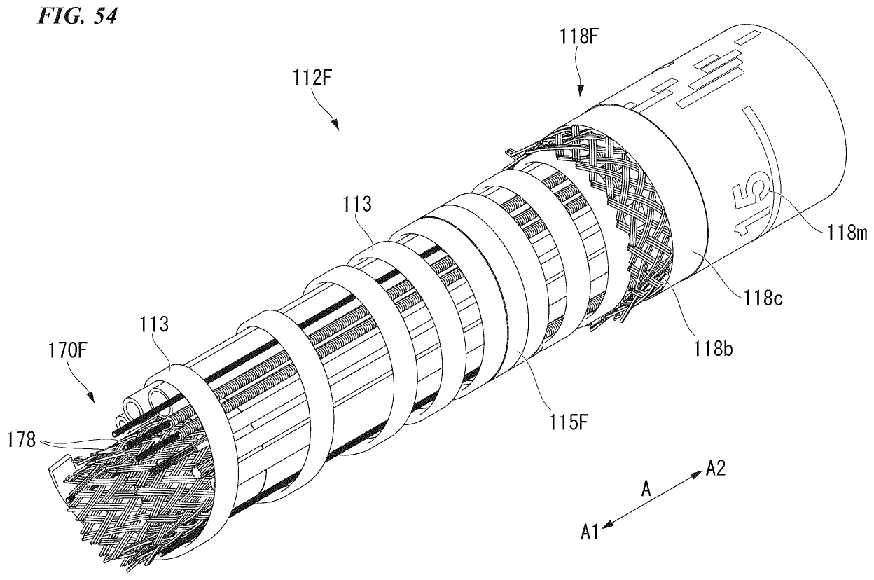

FIG. 54 is a diagram showing the bending portion 112F.

The bending portion 112F includes a spring 113, multiple ring members 115F, and an outer sheath 118F, which is the outermost part. The bending wire 160F and the built-in element 170F are inserted through the internal passage 101 formed in the bending portion 112F.

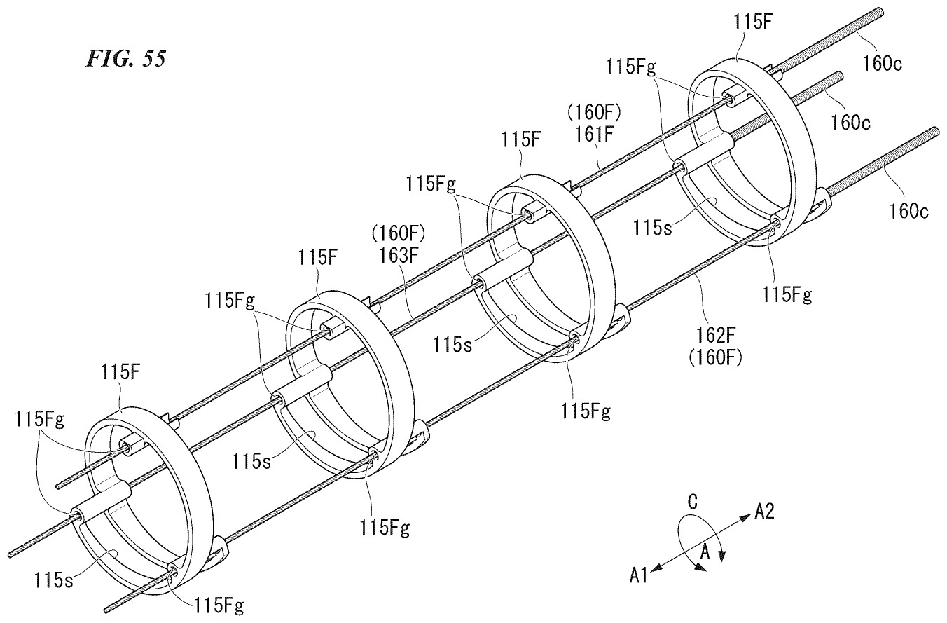

FIG. 55 is a diagram showing the ring member 115F.

Multiple ring members 115F are arranged in the internal passage 101 formed in the bending portion 112F. The multiple ring members 115F are arranged in the axial direction A. The multiple ring members 115F are not connected, and adjacent ring members 115F are arranged at a distance in the axial direction A. The built-in element 170F including the first channel tube 171F is inserted through the multiple ring members 115A. The bending wire 160F is inserted through the wire-guide 115Fg formed in the ring member 115F.

The ring member 115F includes a slit 115s formed therein into which the spring 113 is fitted. The ring member 115F is attached to the spring 113 by fitting the spring 113 into the slit 115s.

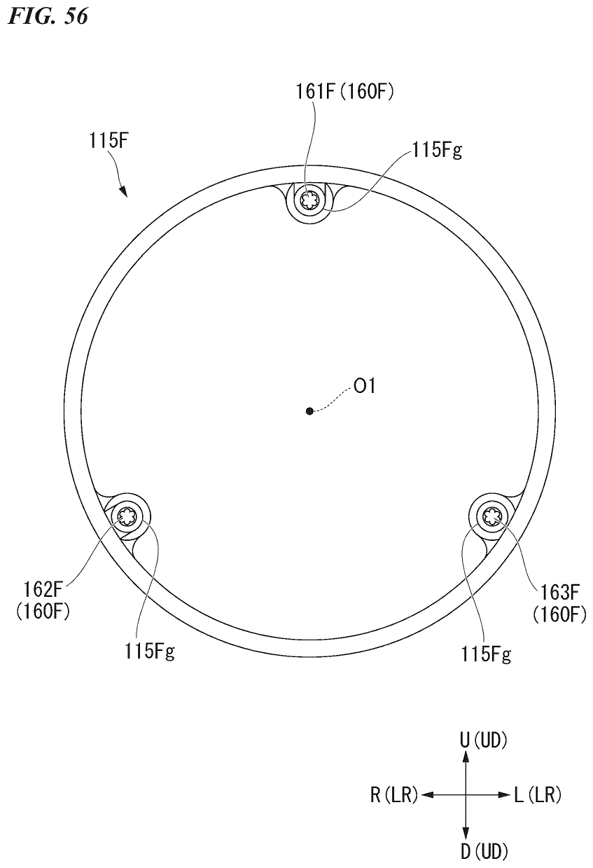

FIG. 56 is a diagram showing the ring member 115F as viewed from the longitudinal direction A.

Three wire-guides 115Fg are provided on the inner peripheral surface of the ring member 115F. The three wire-guides 115Fg are arranged at equal intervals along the circumferential direction C. The three wire-guides 115Fg are arranged at intervals of 120 degrees with respect to the central axis O1 in the longitudinal direction A.

The bending wire 160F is a wire that bends the bending portion 112F. The bending wire 160F passes through the internal path 101 and extends to the attachment portion 150. The bending wire 160F has a first bending wire 161F, a second bending wire 162F, and a third bending wire 163F. The number of bending wires 160F is three, not four. That is, the bending wire 160F is composed of three wires, a first bending wire 161F, a second bending wire 162F, and a third bending wire 163F.

The direction perpendicular to the longitudinal axis of the first bending wire 161F inserted through the wire-guide 115Fg and the central axis O1 of the longitudinal direction A is the up-down direction (also called the “UD direction”). The direction perpendicular to the longitudinal direction A and the up-down direction is the left-right direction (also called the “LR direction”). Although the number of bending wires 160F is three, the bending wires 160F can bend the bending portion 112F in all directions, including the up-down direction and the left-right direction.

The bending wire 160F may be inserted through a coil sheath 160c, as shown in FIG. 55. However, the distal end of the bending wire 160F that passes through the multiple ring members 115F does not have to pass through the coil sheath 160c. Since there is no coil sheath 160c in the bending portion 112F where the ring members 115F are arranged, the coil sheath does not suppress compressive deformation due to wire traction, and the outer sheath 118F can be smoothly bent.

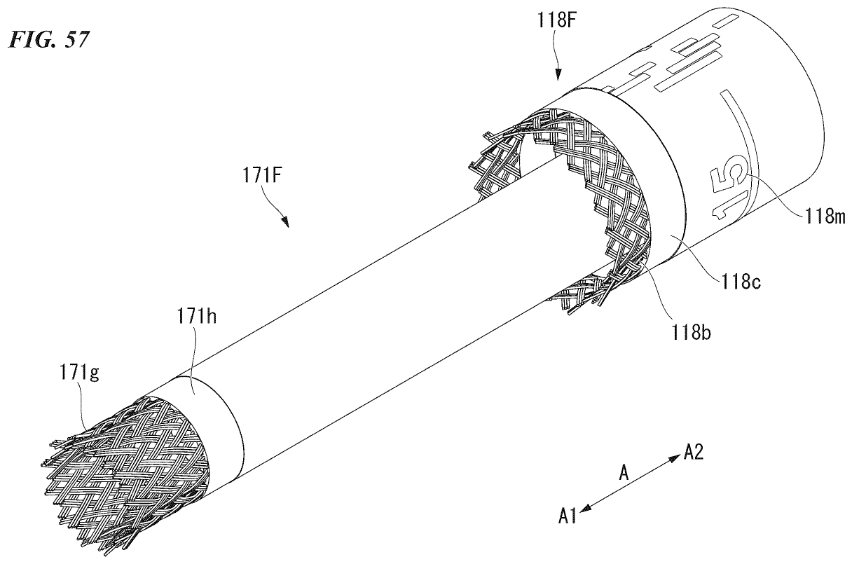

FIG. 57 is a diagram showing the first channel tube 171F.

The first channel tube 171F is a tube having a large-diameter first treatment tool lumen 171r. The inner diameter D1 of the first treatment tool lumen 171r is larger than the inner diameter D2 of the second treatment tool lumen 172r. Specifically, the inner diameter D1 of the first treatment tool lumen 171r can be three to five times the inner diameter D2 of the second treatment tool lumen 172r. As shown in FIG. 3, two treatment manipulators 400 can be inserted into the first treatment tool lumen 171r.

The inner diameter D1 of the first treatment tool lumen 171r can be set to ½ or more of the outer diameter of the outer sheath 118. Even in an existing endoscope (e.g., a transnasal endoscope) with a relatively large inner diameter of the treatment tool channel, the inner diameter of the treatment tool channel is about 2.4 mm (about ⅖ of the outer diameter) compared to an outer diameter of about 6 mm. Compared to an existing endoscope, the insertion manipulator 100F has a larger inner diameter D1 of the first treatment tool lumen 171r compared to the outer diameter of the outer sheath 118. Therefore, a large treatment manipulator 400F can be inserted into the first treatment tool lumen 171r, expanding the range of procedures.

The first channel tube 171F includes a blade tube 171g and a coating 171h arranged on both the inner and outer circumferential sides of the blade tube 171g. The coating 171h arranged on the outer periphery is also called the outer coating. The coating 171h may be either an inner coating or an outer coating.

The braided tube 171g is a tube in which metal wires or resin wires are woven into a braid shape. The braided tube 171g is made of, for example, aramid or UHMW-PE (Ultra-High-Molecular-Weight Polyethylene).

The coating 171h is sandwiched between the inner and outer circumferential sides of the braided tube 171g, sealing (encapsulating) the braided tube 171g. The coating 171h is attached to the braided tube 171g by coating, co-extrusion, over-casting, or over-molding. The coating 171h is made of a low-friction, low-durometer resin or the like.

The braided tube 171g of the first channel tube 171F is sealed (encapsulated) by the coating 171h. Therefore, the first channel tube 171F has good rigidity without increasing the diameter. Because the coating 171h has low friction and a low durometer, the first channel tube 171F has suitable elasticity and can achieve both durability and ease of bending.

### [Treatment Manipulator 400F]

FIG. 58 shows the treatment manipulator 400F protruding from the first opening 111a. The treatment manipulator 400F is a device that protrudes from the first opening 111a by inserting, for example, the first channel tube 171F of the insertion manipulator 100F, and is inserted into a patient's lumen to treat the affected area. An end effector (treatment portion) for treating the affected area may be disposed at the distal end of the treatment manipulator 400F, and a treatment tool equipped with an end effector may be insertable into a channel provided in the treatment manipulator 400F. The treatment manipulator 400B is driven by a drive unit (actuator) 550 or the like.

FIGS. 59, 60, and 61 show the treatment manipulator 400F.

The treatment manipulator 400F includes a manipulator flexible portion 417, a bendable treatment instrument arm 410F, and treatment instruments (forceps 420, high-frequency knife 430, local injection needle 440, basket 450, etc.) that are inserted through the treatment instrument arm 410F. The covering member that covers the treatment instrument arm 410F is omitted from FIG. 59 and other figures.

The manipulator flexible portion 417 is a long member that extends in the longitudinal direction A and can be inserted into the first channel tube 171F, etc. Two treatment instrument arms 410F are provided on the distal end surface 417a of the manipulator flexible portion 417. The two treatment instrument arms 410F extend from the distal end surface 417a toward the distal end side A1.

The two treatment instrument arms 410F protrude from the first opening 111a of the distal end 111 of the insertion manipulator 100F while being aligned in the left-right direction (LR direction). At least a part of the two treatment instrument arms 410F can be inserted through the notch portion 111n and can be spread outward in the left-right direction. Therefore, by spreading the two treatment instrument arms 410F in the left-right direction, the surgeon can easily treat the affected area placed near the scope 200 with the treatment instrument.

By moving or rotating the insertion manipulator 100F with the two treatment instrument arms 410F in contact with the notch portion 111n, the two treatment instrument arms 410F move or rotate following the movement with their relative positions to the scope 200 fixed. Therefore, even when the insertion manipulator 100F is moved or rotated, the surgeon can easily grasp the position of the treatment instrument inserted through the treatment instrument arm 410F. If the notch portion 111n is a groove into which the treatment arm 410F fits, the relative position of the treatment arm 410F with respect to the scope 200 is fixed, and the position of the treatment arm 410F imaged in the field of view does not change even if the insertion manipulator 100F is moved or rotated.

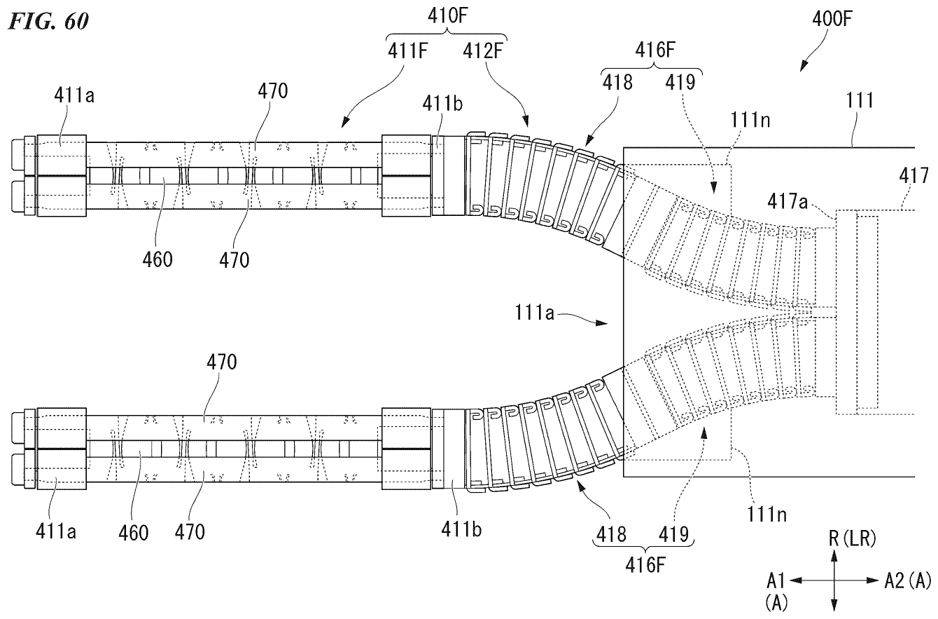

The treatment arm 410F is a hollow, long member and has an insertion path (channel) through which the treatment tool is inserted. The treatment arm 410F includes a first bending portion 411F provided on the distal end side A1 and a second bending portion 412F provided on the proximal end side A2. In the usage state of the treatment manipulator 400F as shown in FIG. 60, the first bending portion 411F can be disposed on the distal end side A1 from the distal end surface of the distal end portion 111. A part of the second bending portion 412F can protrude from the distal end portion 111 to the distal end side A1.

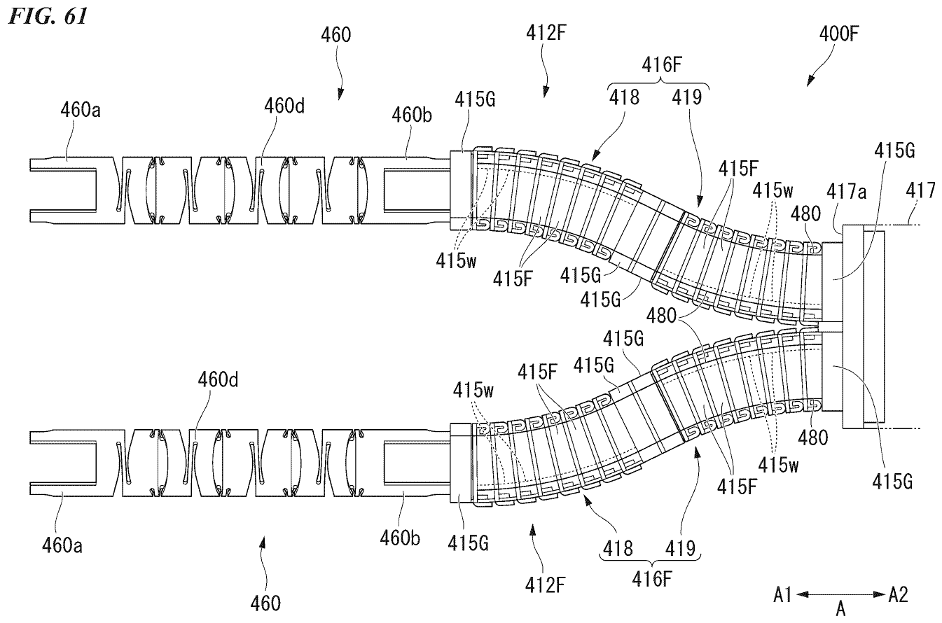

The first bending portion 411F includes a distal end 411a, a proximal end 411b, a bendable bending tube 460, and four artificial muscles 470. The first bending portion 411F can be bent in the up-down and left-right directions by being driven by the artificial muscles 470. The bending tube 460 and the artificial muscles 470 extend along the longitudinal direction A and are arranged side by side. The distal end 411a, the proximal end 411b, and the artificial muscles 470 are omitted from the illustration in FIG. 61.

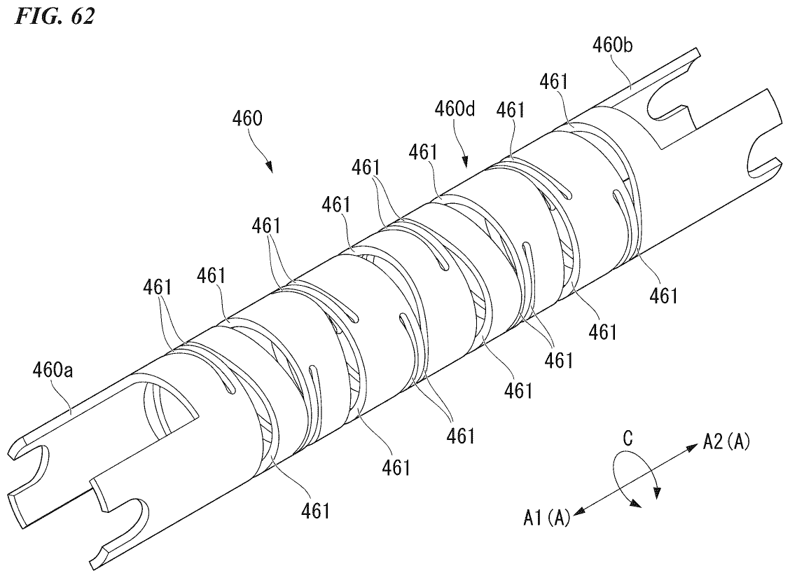

FIG. 62 is a perspective view of the bending tube 460.

The bending tube 460 is a bendable tubular member. The internal space of the bending tube 460 is an insertion path (channel) through which a treatment tool is inserted. The bending tube 460 includes a distal end 460a connected to the distal end 411a, a proximal end 460b connected to the proximal end 411b, and a bending tube main body 460d sandwiched between the distal end 460a and the proximal end 460b.

The bending tube main body 460d is a tubular member formed of metal, resin, or the like, and has a plurality of grooves 461 formed along the circumferential direction C. The length of the grooves 461 in the circumferential direction C may be 70% to 80% of the entire circumference of the bending tube main body 460d. The grooves 461 penetrate the bending tube main body 460d in the radial direction. The bending tube main body 460d is bending by the grooves 461 widening and narrowing.

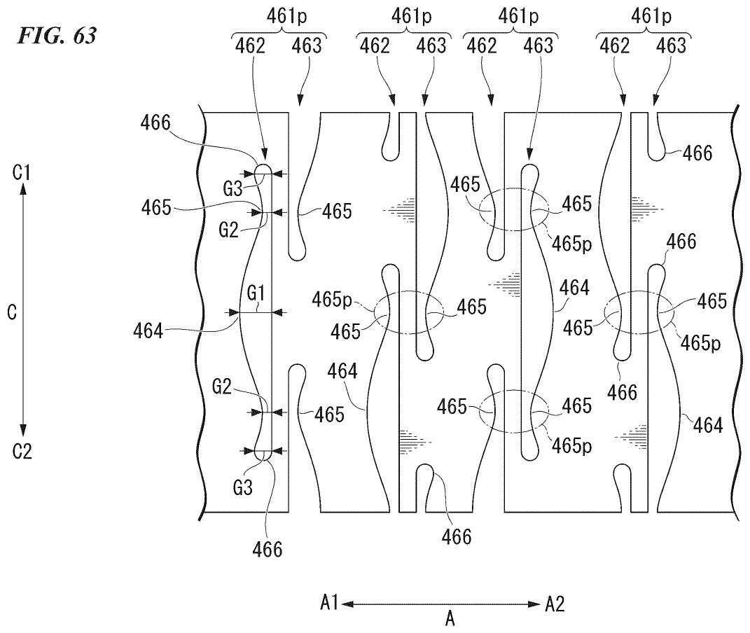

FIG. 63 is an expanded view of the bending tube 460 expanded in the circumferential direction C.

The groove (cut) 461 has a first groove 462 and a second groove 463. The first groove 462 and the second groove 463 are both grooves along the circumferential direction C. The first grooves 462 and the second grooves 463 are alternately arranged along the longitudinal direction A. A set of adjacent first grooves 462 and second grooves 463 is also referred to as a “pair of grooves 461p”. In the pair of adjacent grooves 461p, the pair of grooves 461p on the distal end side A1 is positioned at a position rotated 90 degrees with respect to one side C1 of the circumferential direction C compared to the pair of grooves 461p on the proximal side A2.

The first groove 462 and the second groove 463 are symmetrical with respect to a plane perpendicular to the longitudinal direction A, and are positioned at a position rotated 180 degrees with respect to the circumferential direction C. The first groove 462 is a groove that bulges toward the distal end side A1. The second groove 463 is a groove that bulges toward the proximal end side A2.

The first groove 462 and the second groove 463 may have a first gap 464 in the center in the circumferential direction C, a second gap 465 on both sides of the first gap 464 in the circumferential direction C, and a third gap 466 on the outside of the second gap 465. The length G1 in the longitudinal direction A of the first gap 464 may be longer than the length G2 in the longitudinal direction A of the second gap 465 and the length G3 in the longitudinal direction A of the third gap 466. In addition, the length G2 in the longitudinal direction A of the second gap 465 may be shorter than the length G1 in the longitudinal direction A of the first gap 464 and the length G3 in the longitudinal direction A of the third gap 466 (G1>G3>G2).

In a pair of grooves 461p (first groove 462 and second groove 463), the position of the second gap 465 of the first groove 462 and the position of the second gap 465 of the second groove 463 in the circumferential direction C may be substantially the same. In a pair of grooves 461p (first groove 462 and second groove 463), the second gap 465 of the first groove 462 and the second gap 465 of the second groove 463 may be arranged adjacent to each other along the longitudinal direction A. The adjacent second gaps 465 in a pair of grooves 461p are also referred to as a “pair of second gaps 465p.” The pair of second gaps 465p of a pair of grooves 461p and the first gaps 464 of the grooves 461 of the adjacent pair of grooves 461p may be arranged alternately in the longitudinal direction A.

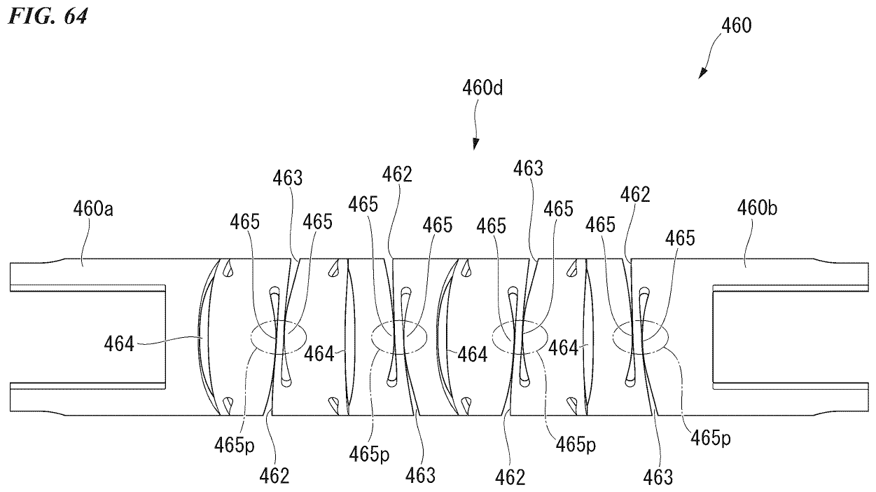

FIG. 64 shows a bending tube 460 compressed at the start of treatment. As shown in FIG. 64, the bending tube 460 starts treatment in a state where it is compressed in the longitudinal direction A until both ends of the second gap 465 in the longitudinal direction A almost touch. At this time, both ends of the first gap 464 in the longitudinal direction A do not come into contact. This state is called the “initial state.” By starting treatment from a state where the surrounding artificial muscle 470 is moderately compressed, it is possible to further compress the artificial muscle 470 during treatment, and it is also possible to further extend the artificial muscle 470 during treatment. The bending tube 460 does not need to be compressed at the time of attachment to the first bending portion 411F, and both ends of the first gap 464 and the second gap 465 may not come into contact.

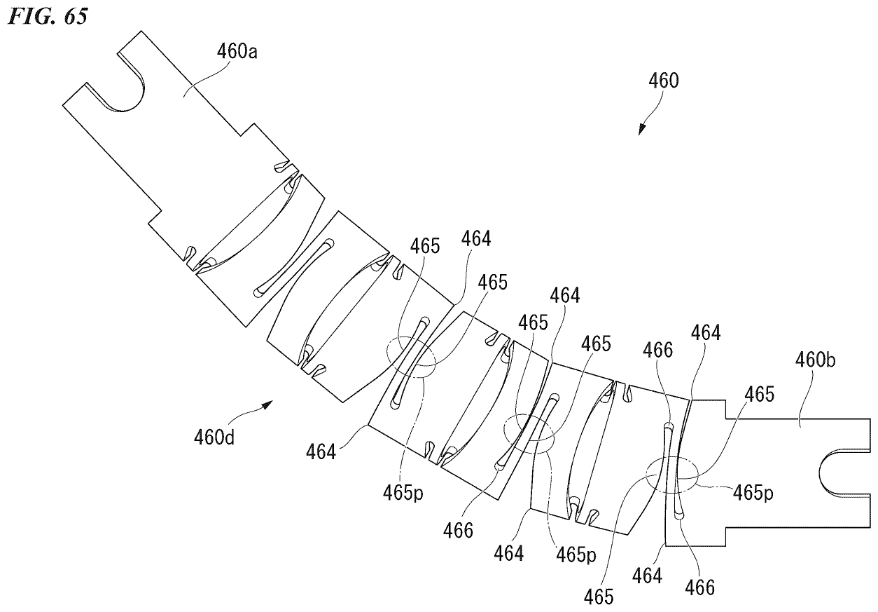

FIG. 65 is a diagram showing a bending tube 460.

The pair of second gaps 465p function as hinges to smoothly bend the bending tube 460. The first gap 464 arranged on the outer side of the bending widens, and the first gap 464 arranged on the inner side of the bending narrows. Since the first gap 464 and the pair of second gaps 465p are evenly arranged in the circumferential direction C, the bending tube 460 can be bending in all directions including up, down, left, and right. When the groove 461 has the third gap 466 at both ends in the circumferential direction C, the bending tube 460 has the effect of dispersing stress when bending.

The bending tube 460 is a tubular member consisting of a combination of thin struts and thick struts. The thin struts are, for example, the portions sandwiched between the pair of second gaps 465p. The thick struts are, for example, the portions sandwiched between the pair of second gaps 465p and the first gap 464. The thin struts function as springs in the compression direction of the artificial muscle 470. The thick struts function as joints when bending.

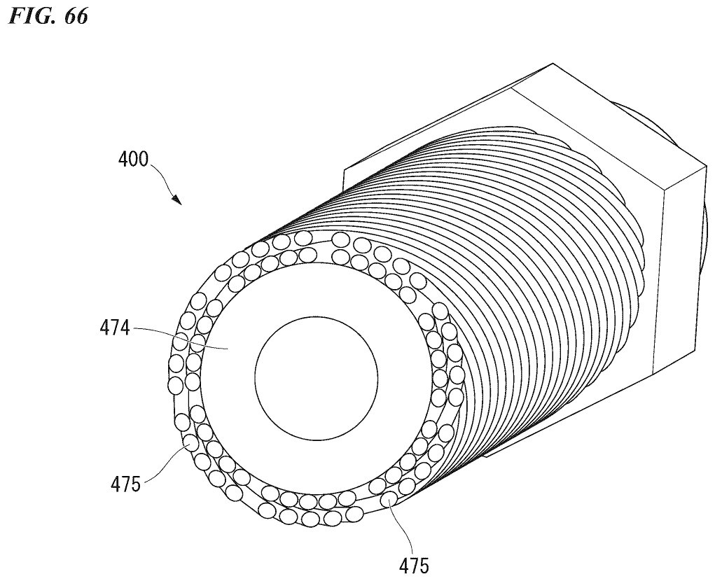

FIG. 66 is a diagram showing the artificial muscle 470.

The artificial muscle 470 is, for example, a Mckibben type artificial muscle, as in the fourth embodiment. A tube 471 is attached to the artificial muscle 470, which supplies a fluid for operating the artificial muscle 470. The artificial muscle 470 contracts with the fluid supplied from the tube 471. The artificial muscle 470 illustrated in FIG. 66 includes a silicone tube 474 and a braided tube 475 arranged on the outside of the silicone tube 474. Fluid is supplied to the silicone tube 474 from the tube 471. The artificial muscle 470 may have a silicone coating on the outer periphery of the braided tube 475.

The four artificial muscles 470 are arranged around the bending tube 460 along the longitudinal direction of the bending tube 460. The distal end of the bending tube 460 and the distal end of the artificial muscle 470 are connected to the distal end portion 411a. The proximal end of the bending tube 460 and the proximal end of the artificial muscle 470 are connected to the proximal portion 411b. When the artificial muscle 470 bends, the bending tube 460 passively bends. The number of artificial muscles 470 is not limited to four. For example, as shown in FIG. 43, the number of artificial muscles 470 may be three.

The distal end 411a and the proximal end 411b may be formed in a rectangular shape extending in the up-down direction when viewed from the longitudinal direction A. Two artificial muscles 470 may be arranged on the upper side (U side) of the bending tube 460, and two artificial muscles 470 may be arranged on the lower side (D side) of the bending tube 460. Therefore, the dimension in the left-right direction of the first bending portion 411F may be shorter than the dimension in the up-down direction. Therefore, it is easy to arrange the two first bending portions 411F side by side in the left-right direction. Furthermore, the bending tube 460 is less likely to interfere with the artificial muscles 470 when bending in the left-right direction compared to when bending in the up-down direction. Therefore, the movable range in the left-right direction of the two first bending portions 411F can be widened.

The artificial muscle 470 may be silicone-coated. In this case, even if there is a bias or variation in the structure of the blade tube 475, the silicone coating absorbs the variation in deformation, realizing uniform deformation of the entire artificial muscle 470. In addition, even if the artificial muscle 470 repeatedly contracts and relaxes, the deviation of the blade tube 475 can be suppressed and the restoration to the original shape is improved, so the reproducibility and stability of the operation of the artificial muscle 470 is improved. Therefore, the operator can easily control the bending operation of the artificial muscle 470 in the up, down, left, and right directions.

The operation of the artificial muscle 470 is divided into a “nonlinear region” in which the amount of deformation relative to the amount of pressurization of the fluid is nonlinear, and a “linear region” in which the amount of change relative to the amount of pressurization of the fluid is linear or can be handled as approximately linear in terms of the control of the manipulator. When the fluid is pressurized from a non-pressurized state, the operation of the artificial muscle 470 transitions to the linear region via the nonlinear region. Therefore, the artificial muscle 470 is in the “initial state” when the fluid is compressed by pressurizing it until it transitions to the linear region, the responsiveness of the artificial muscle 470 improves, and the controllability of the first bending portion 411F improves.

The first bending portion 411F is driven by the artificial muscle 470, not by a wire. Therefore, there is no need to insert a wire into the internal space of the bending tube 460, and sufficient space can be secured for inserting the treatment tool. In addition, there is no need to provide a path for inserting the wire in the bending tube 460, and the structure of the bending tube 460 can be simplified. In addition, since wire breakage does not occur, maintenance is easy.

As shown in FIG. 60, the second bending portion 412F has a shoulder joint 416F that greatly bends the treatment tool arm 410F in the left-right direction (LR direction), and a wire 480. The shoulder joint 416F bends the second bending portion 412F into an S-shape when viewed from the up-down direction (UD direction). As shown in FIG. 59, the distal ends of the second bending portions 412F of the two treatment instrument arms 410F are bent in the left-right direction and move away from each other.

As shown in FIG. 61, the wire 480 is attached to the second bending portion 412F. The second bending portion 412F is bent by pulling the second bending portion 412F from the proximal end side A2. Two wires 480 are attached to one second bending portion 412F.

As shown in FIG. 61, the shoulder joint 416F has a first shoulder joint 418 provided on the distal end side A1 and a second shoulder joint 419 provided on the proximal end side A2.

The first shoulder joint 418 is connected to the distal end of the second shoulder joint 419 and extends to the distal end side A1. The first shoulder joints 418 of the two treatment instrument arms 410F are bending in a direction approaching each other (inward) in the left-right direction (LR direction).

The second shoulder joint 419 is connected to the distal end surface 417a of the manipulator flexible portion 417 and extends to the distal end side A1. The second shoulder joints 419 of the two treatment instrument arms 410F are bending in a direction moving away from each other (outward) in the left-right direction (LR direction).

The first shoulder joint 418 and the second shoulder joint 419 are bending in opposite directions in the left-right direction, so that the second bending portion 412F is bending in an S-shape.

As shown in FIG. 61, the shoulder joint 416F has a bending piece 415F and a connecting piece 415G. The first shoulder joint 418 and the second shoulder joint 419 have connecting pieces 415G arranged at the distal end and proximal end, and multiple bending pieces 415F are arranged between the two connecting pieces 415G. When the wire 480 is pulled, adjacent bending pieces 415F come into contact with each other.

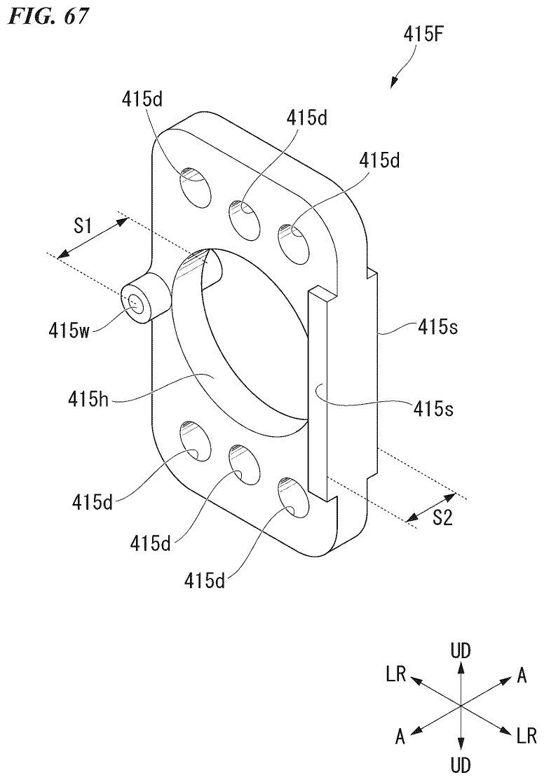

FIG. 67 is a diagram showing the bending piece 415F.

The bending piece 415F may be formed in a substantially rectangular parallelepiped shape, or may be in a rectangular shape extending in the up-down direction as viewed from the longitudinal direction A. The bending piece 415F has an insertion hole 415h that penetrates in the longitudinal direction A at the center. The insertion hole 415h is an insertion path (channel) for a treatment tool. The bending piece 415F has a wire hole 415w at one end in the left-right direction, and a spacer 415s at the other end in the left-right direction. The wire hole 415w is formed along the longitudinal direction A, and is a wire-guide through which the wire 480 passes. The spacer 415s protrudes on both sides in the longitudinal direction A. On the upper and lower sides of the insertion hole 415h, there are provided connection holes 415d through which the connection members 415c (see FIG. 59) that connect the bending pieces 415F and the connecting pieces 415G pass.

When adjacent bending pieces 415F come into contact, the wire holes 415w come into contact with each other, and the spacers 415s come into contact with each other. The length S1 of the wire hole 415w in the longitudinal direction A is longer than the length S2 of the spacer 415s in the longitudinal direction A (S1>S2). Therefore, when the wire 480 is pulled, the adjacent bending pieces 415F come into contact with each other and bend in the left-right direction.

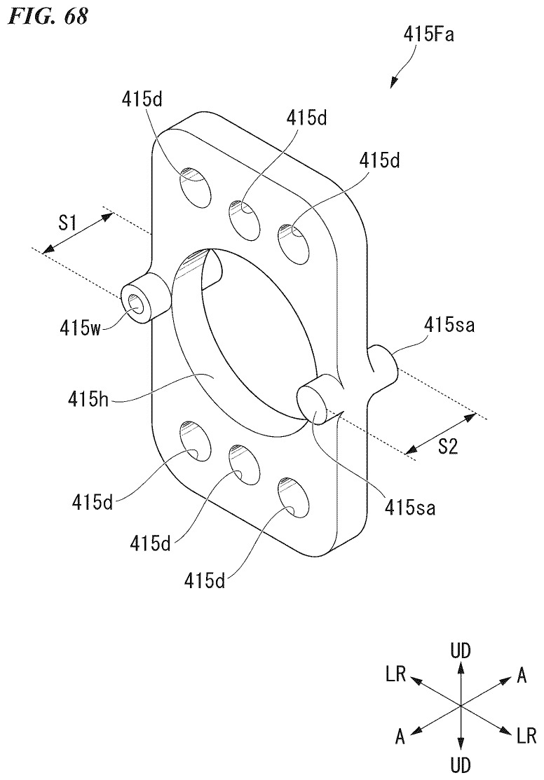

FIG. 68 is a diagram showing bending piece 415Fa, which is a modified example of bending piece 415F.

The bending piece 415Fa includes a spacer 415sa, which is a modified example of spacer 415s. The spacer 415sa is formed in a cylindrical shape. The spacer 415sa is provided in the center in the up-down direction (UD direction).

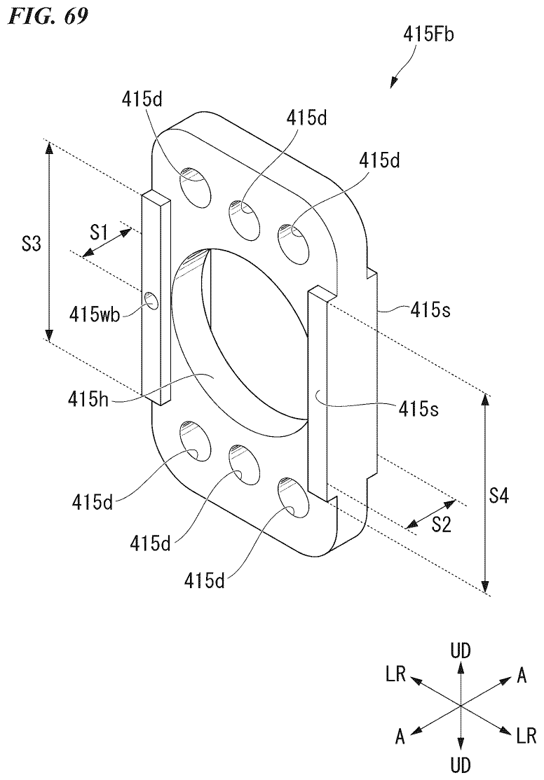

FIG. 69 is a diagram showing a bending piece 415Fb, which is a modified example of bending piece 415F.

The bending piece 415Fb includes a wire hole 415wb, which is a modified example of wire hole 415w. The wire hole 415wb is formed in a rectangular shape extending in the up-down direction (UD direction) when viewed from longitudinal direction A. In the wire hole 415wb, the hole through which wire 480 is inserted is provided in the center in the up-down direction. Since the wire hole 415wb extends in the vertical direction, it is possible to prevent the bending piece 415Fb from wobbling in the vertical direction.

The vertical length S3 of the wire hole 415wb is preferably shorter than the vertical length S4 of the spacer 415s (S3<S4). The vertical length 3 of the wire hole 415wb on the force point side of the wire 480 is short, and the vertical length S4 of the spacer 415s on the side farther from the force point side of the wire 480 is long, so that the bending operation is stable.

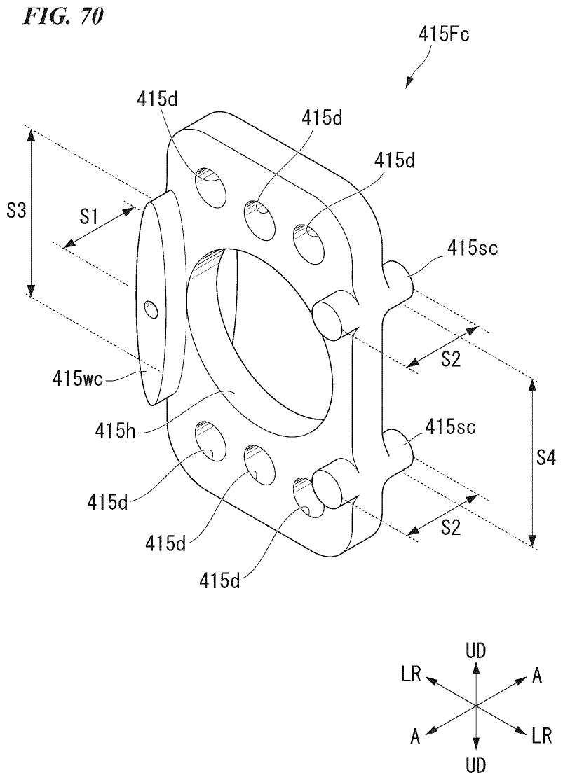

FIG. 70 is a diagram showing a bending piece 415Fc, which is a modified example of the bending piece 415F.

The bending piece 415Fc has a wire hole 415wc, which is a modified example of the wire hole 415w. The wire hole 415wc is formed in an elliptical shape extending in the vertical direction (UD direction) when viewed from the longitudinal direction A. The hole through which the wire 480 passes in the wire hole 415wc is provided in the center in the vertical direction. Because the wire hole 415wc extends in the vertical direction, it is possible to prevent the bending piece 415Fc from wobbling in the vertical direction.

The bending piece 415Fc includes a spacer 415sc, which is a modified example of the spacer 415s. The spacer 415sc is formed in a cylindrical shape and is provided on both sides in the vertical direction.

It is desirable that the vertical length S3 of the wire hole 415wc is shorter than the vertical length S4 of the spacer 415sc (S3<S4). The bending operation is stabilized by having a short vertical length 3 of the wire hole 415wc on the force point side of the wire 480 and a long vertical length S4 of the spacer 415sc on the side farther from the force point side of the wire 480.

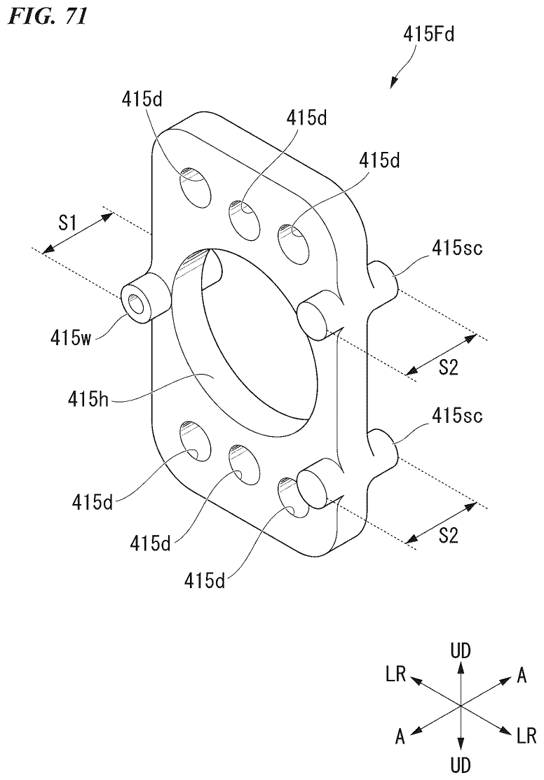

FIG. 71 is a diagram showing a bending piece 415Fd, which is a modified example of bending piece 415F.

The bending piece 415Fd includes spacer 415sc, which is a modified example of spacer 415s. The spacer 415sc is formed in a cylindrical shape and is provided on both the top and bottom sides.

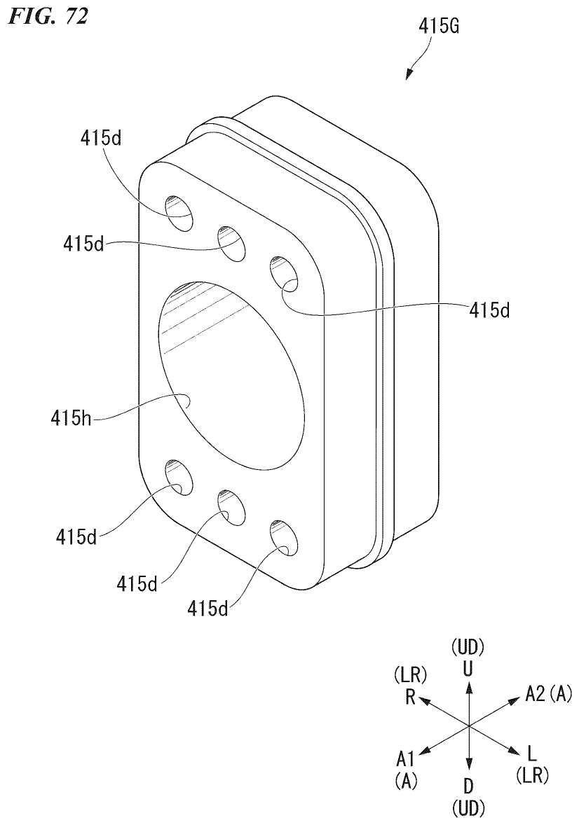

FIG. 72 is a diagram showing a connecting piece 415G.

The connecting piece 415G may be formed in a substantially rectangular parallelepiped shape, or may be in a rectangular shape extending in the top and bottom direction when viewed from the longitudinal direction A. The bending piece 415F includes an insertion hole 415h that penetrates in the longitudinal direction A at the center. The insertion hole 415h is an insertion path (channel) for a treatment tool. At the upper and lower sides of the insertion hole 415h, there are provided connection holes 415d through which the connection members 415c (see FIG. 59) that connect the bending piece 415F and the connecting piece 415G are inserted.

As shown in FIG. 61, the bending pieces 415F are provided so that the wire holes 415w are located on the outside in the left-right direction in the first shoulder joints 418 of the two treatment instrument arms 410F. The length S1 in the longitudinal direction A of the wire hole 415w located on the outside is longer than the length S2 in the longitudinal direction A of the spacer 415s located on the inside (S1>S2). Therefore, when the wire 480 is pulled, the first shoulder joints 418 of the two treatment instrument arms 410F are bent in a direction approaching each other (inside) in the left-right direction (LR direction).

As shown in FIG. 61, a bending pieces 415F are provided in the second shoulder joints 419 of the two treatment instrument arms 410F so that the wire hole 415w is located on the inside in the left-right direction. The length S1 in the longitudinal direction A of the wire hole 415w located on the inside is longer than the length S2 in the longitudinal direction A of the spacer 415s located on the outside (S1>S2). Therefore, when the wire 480 is pulled, each of the second shoulder joints 419 of the two treatment instrument arms 410F bends in a direction away from each other (outside) in the left-right direction (LR direction).

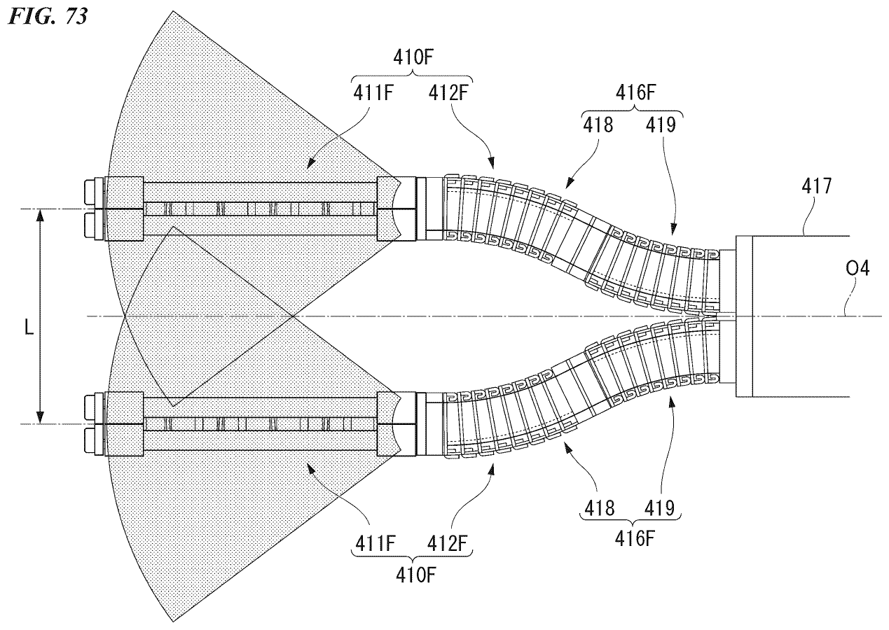

FIG. 73 shows a treatment instrument arm 410F with a bent second bending portion 412F.

As the first shoulder joint 418 and the second shoulder joint 419 bend in opposite directions in the left-right direction, the second bending portion 412F bends in an S-shape. Therefore, the distance (shoulder width) L between the central axes at the distal ends of the second bending portions 412F of the two treatment instrument arms 410F can be widened. When treating a target site using two treatment instrument arms 410F, by appropriately separating the positions of the proximal ends of the two first bending portions 411F, the surgeon S can secure sufficient space for treatment and easily treat the target site. In addition, the configuration in which the artificial muscle 470 can be driven while protruding from the insertion manipulator 100F has the advantage that the artificial muscle 470 can be driven without being limited by the outer diameter of the insertion manipulator 100F. The configuration in which the distal end portion 111 has the notch portion 111n has the advantage that the treatment tool protruding from the distal end of the treatment manipulator 400F can maintain a positional relationship that does not interfere with the treatment tool protruding from the second opening 111b, and the advantage that a positional relationship that does not obstruct the field of view can be maintained. Furthermore, by arranging the artificial muscle 470 at the distal end, it has the advantage that it is less susceptible to changes in shape in the entire length of the treatment manipulator 400F and the insertion manipulator 100F. Furthermore, by selecting a fluid-driven artificial muscle 470, the degree of freedom in arranging the ducts inside the treatment manipulator 400F is high.

The first bending portion 411F is driven to bend by the artificial muscle 470, so the bending angle may be smaller than other bending portions driven by wires or the like. However, as shown in FIG. 73, when the second bending portion 412F is bent into an S-shape, the two first bending portions 411F can be arranged approximately parallel along the longitudinal direction A while being spaced apart in the left-right direction. Therefore, even if the treatment manipulator 400F includes the first bending portion 411F with a relatively small bending angle, it is easy to treat the affected area arranged on the central axis O4 of the treatment manipulator 400F. Furthermore, since the bending angle is relatively small but delicate driving is possible, it is also suitable for treatment in deep areas such as the right colon.

In the treatment manipulator 400F, the treatment tool does not have a bending mechanism, but the treatment tool arm 410F through which the treatment tool is inserted has the bending mechanism. Since the treatment tool advances and retracts relative to the bending treatment tool arm 410F, the treatment manipulator 400F has a wider approachable range than other treatment tools with bending mechanisms. Even the treatment manipulator 400F with the first bending portion 411F, which has a small bending angle, can approach a wider range. In addition, since the treatment tool of the treatment manipulator 400F does not have a bending mechanism, the mechanism can be simplified and maintenance is easy.

### [Driver 500F]

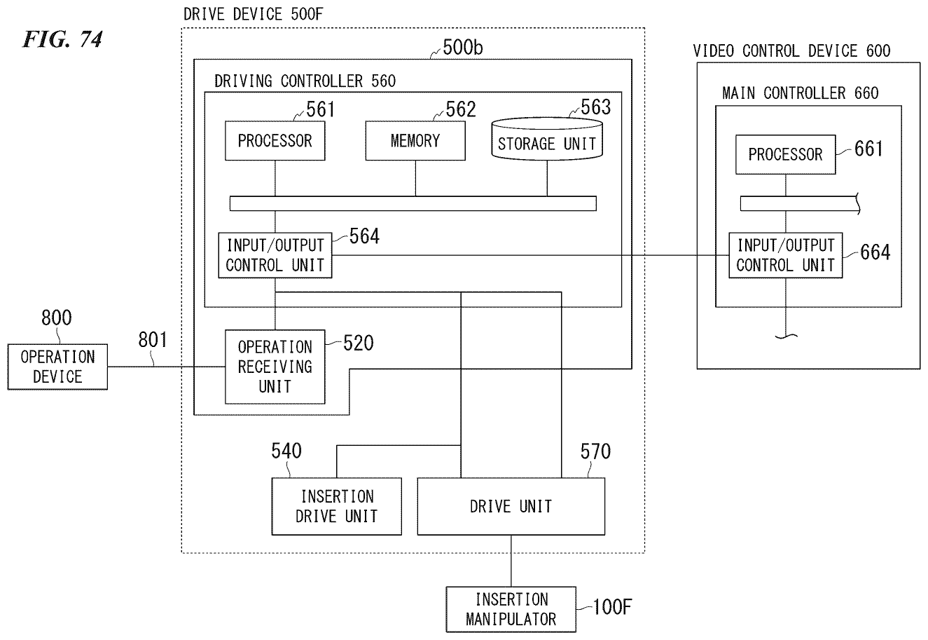

FIG. 74 is a functional block diagram of the driver 500F.

The driver 500F includes an operation receiver 520, a drive controller 560, an insertion drive unit 540, and a drive unit 570. The operation-receiving unit 520 and the drive controller 560 are mounted on the drive device main body 500b. The insertion drive unit 540 and the drive unit 570 are devices separated from the drive device main body 500b. The drive controller 560 controls the insertion drive unit 540 and the drive unit 570. The insertion drive unit 540 and the drive unit 570 are mounted on the cart 500W.

Compared to the drive device 500 of the first embodiment, the drive device 500F is incorporated into a “drive unit 570” which is a device separated from the air supply and a suction drive unit 530 and a drive unit 550.

### [Insertion Drive Unit 540]

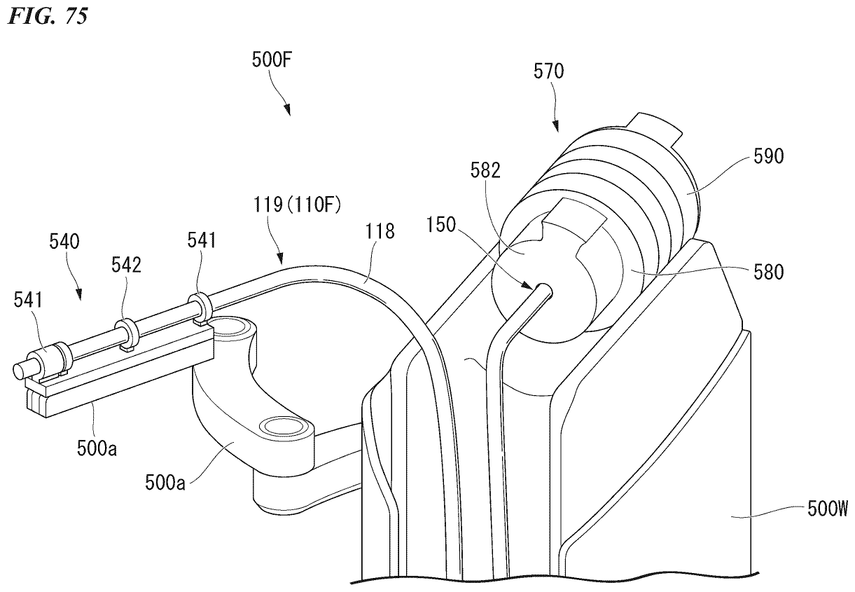

FIG. 75 is a diagram showing the insertion drive unit 540.

The insertion drive unit 540 assists the operation of inserting the insertion manipulator 100F into the body of the patient P. The insertion drive unit 540 is mounted on an arm 500a that extends deformably from the cart 500W. The surgeon S can operate the arm 500a to place the insertion drive unit 540 in a position that allows the insertion manipulator 100F to be easily inserted into the patient P's body. In addition, when the position of the insertion manipulator 100F is to be readjusted (repositioned) during treatment, the insertion drive unit 540 can be used to readjust the position, which has the effect of preventing buckling of the insertion manipulator 100F.

The insertion drive unit 540 has two rings 541 through which the flexible portion 119 of the insertion manipulator 100F is inserted, and a drive unit 542 disposed between the two rings 541.

The drive unit 542 can be fixed by sandwiching the flexible portion 119. The drive unit 542 can move between the two rings 541, and can move the flexible portion 119, which is fixed by clamping it, forward and backward. The drive unit 542 can rotate in the circumferential direction, and can rotate the flexible portion 119, which is fixed by clamping it. The insertion drive unit 540 is not limited to the mode in which the drive unit 542 clamps and fixes the flexible portion 119. For example, the insertion drive unit 540 may be equipped with rollers, and the rollers may be pressed against the flexible portion 119 to fix it, and the flexible portion 119 may be moved forward and backward and rotated by the rotation of the rollers.

### [Drive Unit 570]

FIG. 76 is a diagram showing the drive unit 570.

The drive unit 570 is a device that drives the insertion manipulator 100F and the treatment manipulator 400F. The drive unit 570 is mounted on the top of the cart 500W. The base unit 571 and the drive unit 570 have a first drive unit 580 and a second drive unit 590.

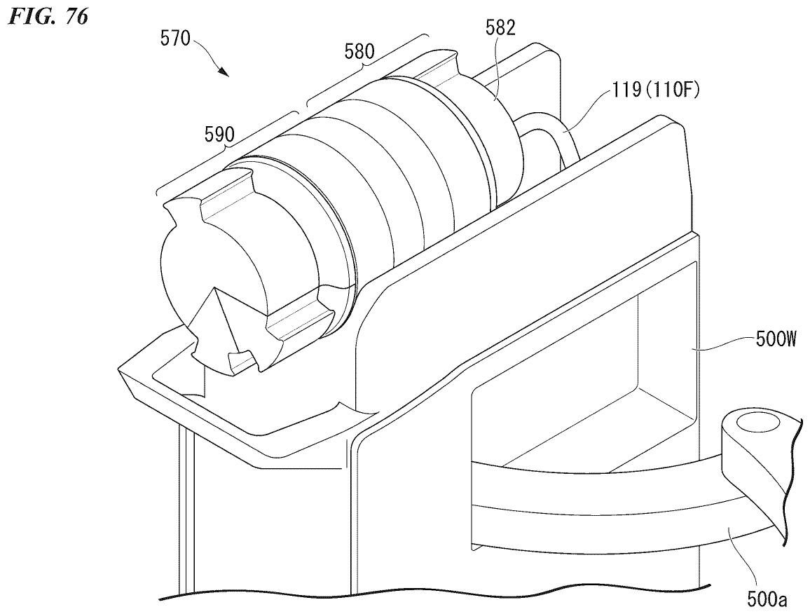

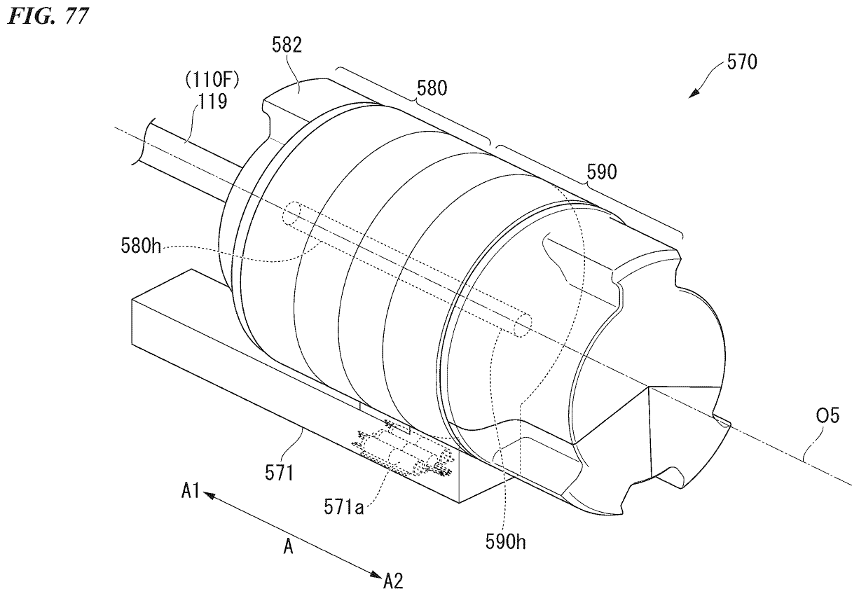

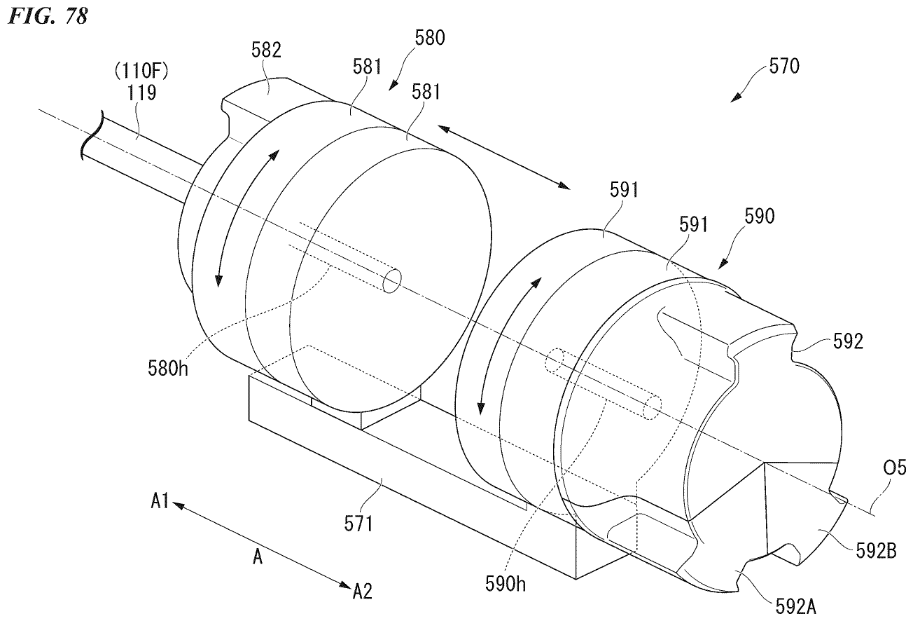

FIGS. 77 and 78 are diagrams showing the operation of the drive unit 570.

The drive unit 570 has a generally cylindrical shape extending in the longitudinal direction A. The first drive unit 580 is disposed on the distal end side A1, and the second drive unit 590 is disposed on the proximal side A2. The base unit 571 supports the first drive unit 580 and the second drive unit 590 so that they can be driven. The base unit 571 has a motor 571a that drives the first drive unit 580 and the second drive unit 590.

The first drive unit 580 is a device that drives the insertion manipulator 100F. The first drive unit 580 is supported by the base unit 571 so as to be movable back and forth in the longitudinal direction A and rotatable about a central axis O5 along the longitudinal direction A. The first drive unit 580 has a first insertion passage 580h that passes through in the longitudinal direction A, through which the treatment manipulator 400F or the hardness-changing device 300 can be inserted.

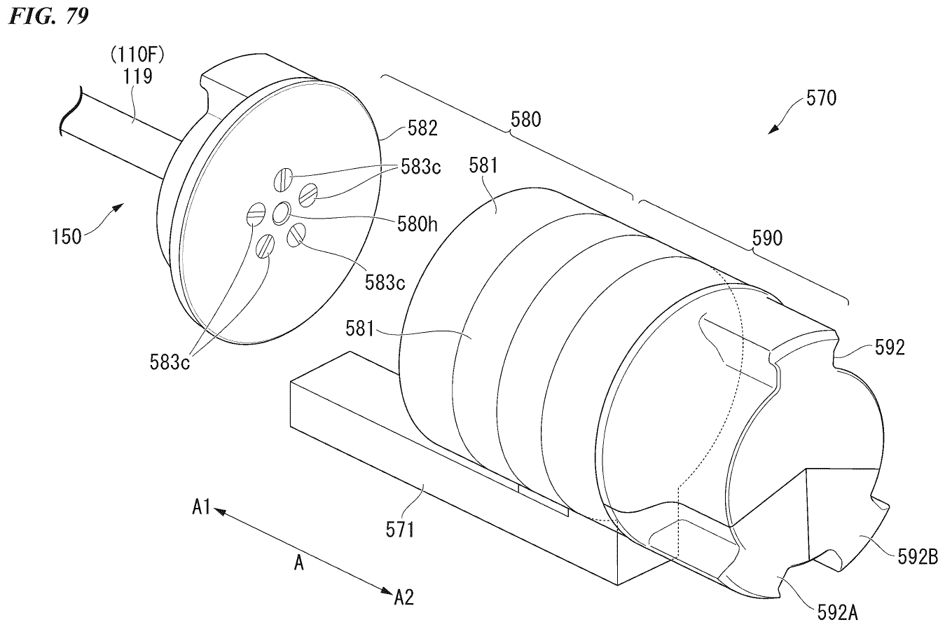

FIG. 79 is an exploded view of the first drive unit 580.

The first drive unit 580 has a first motor unit 581 on the proximal end side A2 and a first adaptor 582 on the distal end side A1.

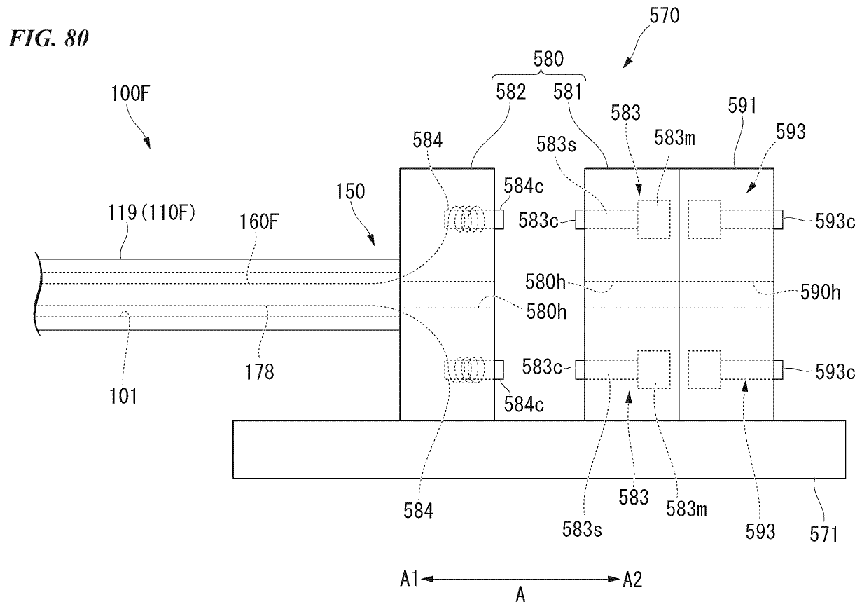

FIG. 80 is a cross-sectional view of the first motor unit 581 from which the first adaptor 582 has been separated.

The first motor unit 581 is a unit to which the first adaptor 582 can be attached from the distal end side A1. The first motor unit 581 drives the first drive unit 584 of the attached first adaptor 582. The first motor unit 581 includes a first drive unit 583.

The first drive unit (first actuator) 583 includes a first motor 583m, a first shaft 583s driven by the first motor 583m, and a first coupled portion 583c connected to the first shaft 583s. The first coupled portion 583c is exposed at the distal end side A1 of the first motor unit 581.

The first adaptor 582 is an adaptor that can be attached to and detached from the distal end side A1 of the first motor unit 581. The distal end side A1 of the first adaptor 582 is connected to the attachment portion 150 provided at the proximal end of the flexible portion 119 of the insertion manipulator 100F. The internal path 101 of the insertion manipulator 100F and the first insertion passage 580h are in communication with each other. The first adaptor 582 has a first drive unit 584.

The first drive unit (driving force transmission part) 584 is a member to which a driving force for driving the bending wire 160F and the scope operation wire 178 is input. The first drive unit 584 is, for example, a rotating drum that rotates about a central axis along the longitudinal direction A. The first drive unit 584 has a first coupling portion 584c. The first coupling portion 584c is exposed to the proximal end side A2 of the first adapter 582. The first drive unit 584 is not limited to a rotating drum. For example, the first drive unit 584 may be a ball screw. Alternatively, an artificial muscle may be used for the bending portion 112F, and the first drive unit 584 may be a cylinder. The cylinder may be a hydraulic cylinder, a water pressure cylinder, or a pneumatic cylinder.

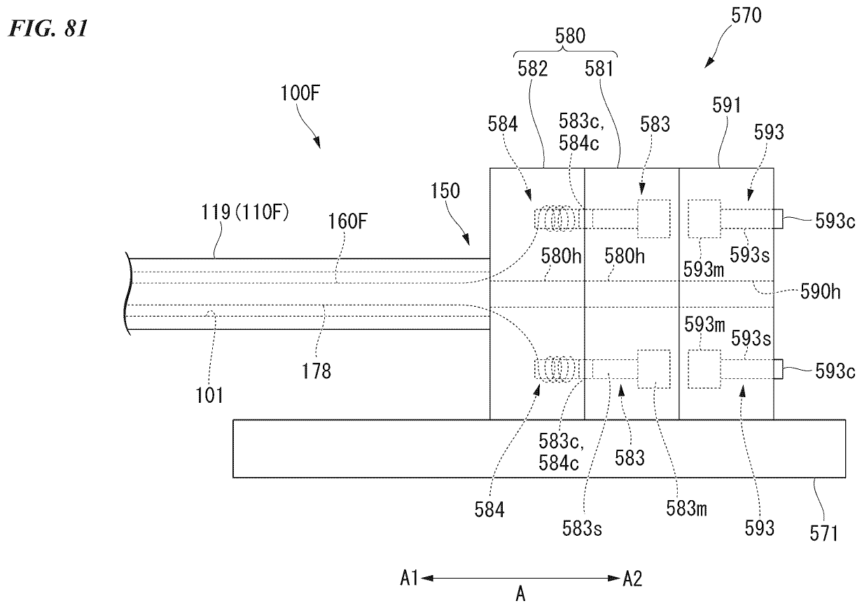

FIG. 81 is a cross-sectional view of the first motor unit 581 to which the first adapter 582 is attached.

When the first adapter 582 is attached to the first motor unit 581, the first coupled portion 583c and the first coupling portion 584c are coupled. As a result, the rotation of the first shaft 583s by the first motor 583m is transmitted to the first drive unit 584. This allows the first drive unit 583 to drive the bending wire 160F and the scope operation wire 178.

The second drive unit 590 is a device that drives the treatment manipulator 400F and the hardness-changing device 300. The second drive unit 590 is supported by the base unit 571 so as to be rotatable about the central axis O5. The second drive unit 590 has a second insertion passage 590h that passes through in the longitudinal direction A, through which the treatment manipulator 400F and the hardness-changing device 300 can be inserted.

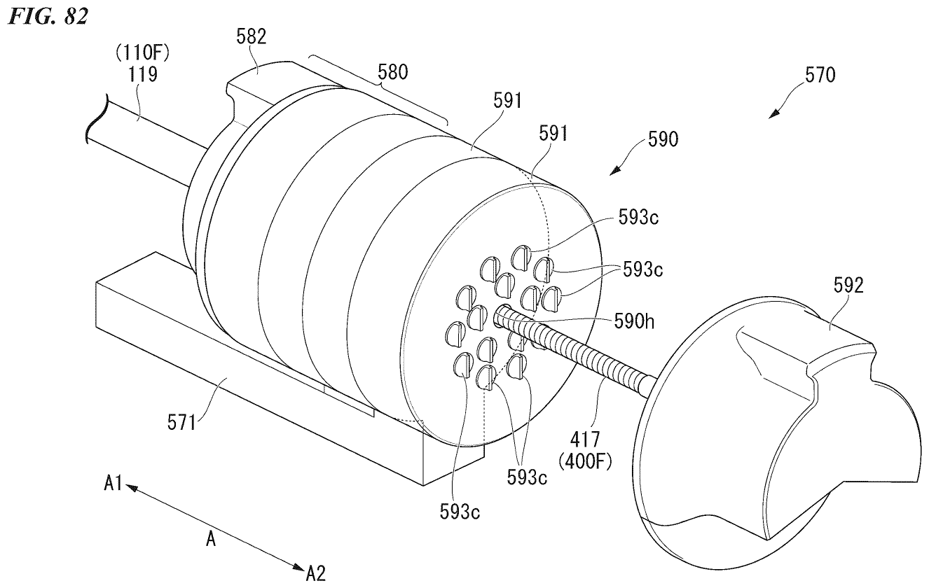

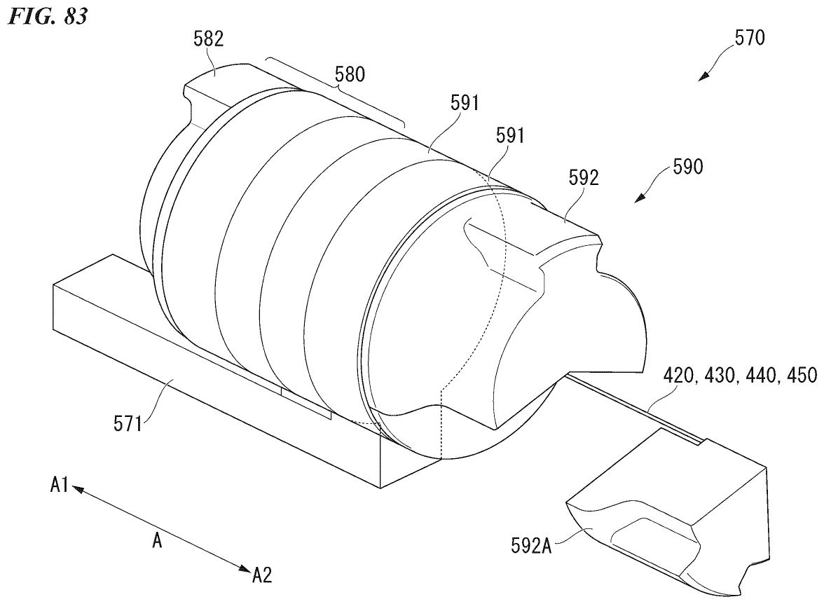

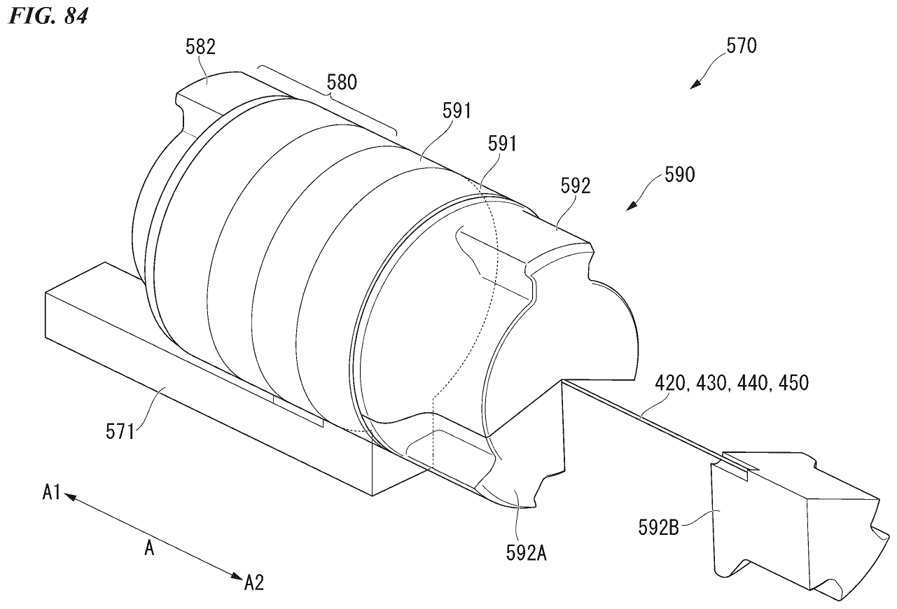

FIGS. 82 to 84 are exploded views of the second drive unit 590. The second drive unit 590 has a second motor unit 591 on the distal end side A1, and a second adaptor 592, a third adaptor 592A, and a fourth adaptor 592B on the proximal side A2.

When the first drive unit 580 moves to the proximal side A2, the first drive unit 580 comes into contact with the second drive unit 590. As shown in FIG. 77, when the first drive unit 580 and the second drive unit 590 come into contact, the first insertion passage 580h and the second insertion passage 590h are connected.

As shown in FIG. 82 to FIG. 84, the second motor unit 591 is a unit to which the second adaptor 592, the third adaptor 592A, and the fourth adaptor 592B can be attached from the proximal side A2. The second motor unit 591 drives the second drive units 594 of the attached second adaptor 592, the third adaptor 592A, and the fourth adaptor 592B. The second motor unit 591 has a second drive unit 593.

As shown in FIG. 81, the second drive unit (second actuator) 593 has a second motor 593m, a second shaft 593s driven by the second motor 593m, and a second coupled portion 593c connected to the second shaft 593s. The second coupled portion 593c is exposed on the proximal end side A2 of the second motor unit 591.

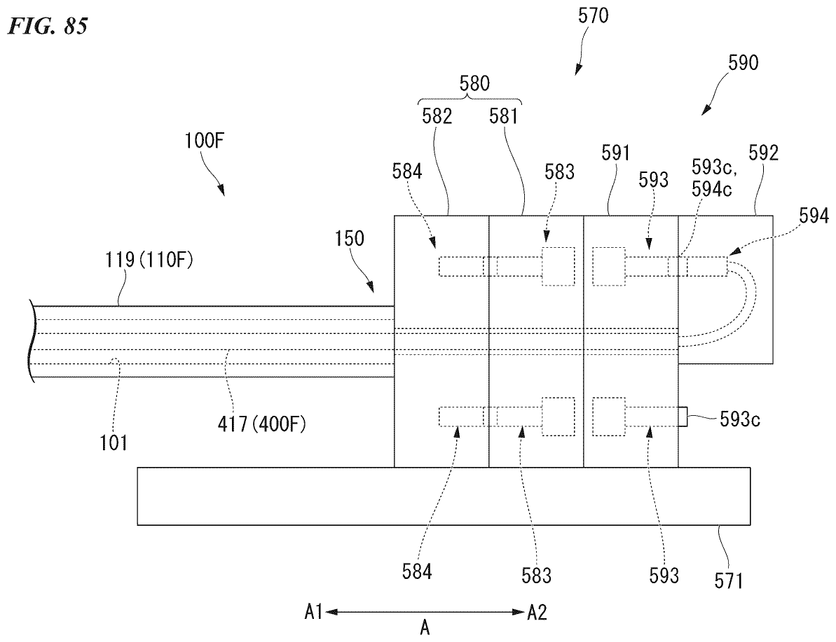

FIG. 85 is a cross-sectional view of the second motor unit 591 to which the second adaptor 592 is attached.

The second adaptor 592 is an adaptor that can be attached and detached to the proximal end side A2 of the second motor unit 591. The treatment manipulator 400F and the hardness-changing device 300 are connected to the distal end side A1 of the second adaptor 592. The treatment manipulator 400F and the hardness varying device 300 are inserted into the internal path 101 of the insertion manipulator 100F via the first insertion passage 580h and the second insertion passage 590h. The second adapter 592 has a second drive unit 594.

The second drive unit (driving force transmission part) 594 is a member to which a driving force is input to drive the tube 471 and wire 480 of the treatment manipulator 400F and the wire 312 of the hardness varying device 300. The second drive unit 594 is, for example, a rotating drum, a cylinder, or a ball screw. The second drive unit 594 has a second coupling portion 594c. The second coupling portion 594c is exposed to the distal end side A1 of the second adapter 592.

When the second adaptor 592 is attached to the second motor unit 591, the second coupled portion 593c and the second coupling portion 594c are coupled. As a result, the rotation of the second shaft 593s by the second motor 593m is transmitted to the second drive unit 594. This allows the second drive unit 593 to drive the tube 471, the wire 480, the wire 312, etc.

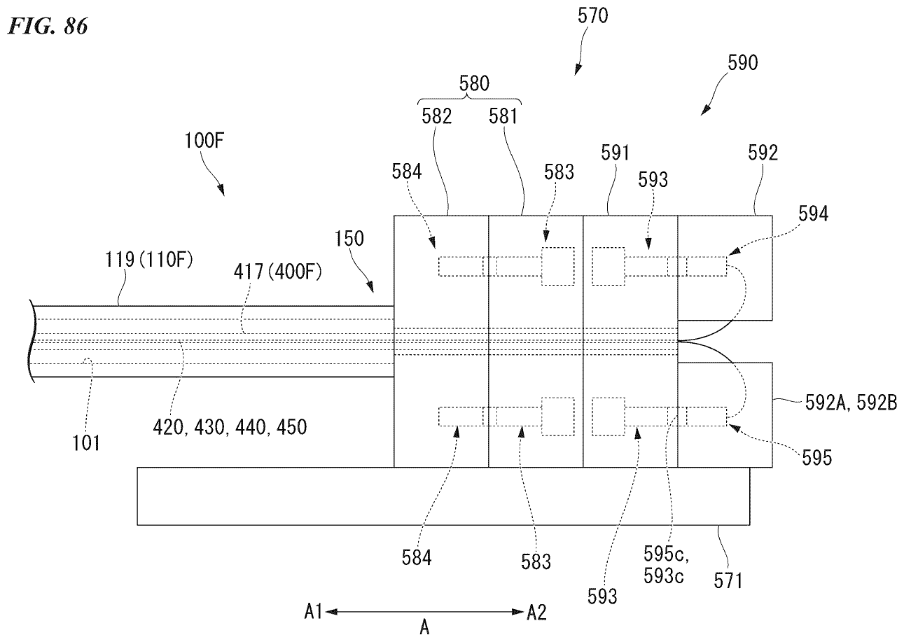

FIG. 86 is a cross-sectional view of the second motor unit 591 to which the third adaptor 592A and the fourth adaptor 592B are attached.

The third adaptor 592A and the fourth adaptor 592B are adaptors that can be attached and detached to the proximal end side A2 of the second motor unit 591. The distal end side A1 of the third adaptor 592A and the fourth adaptor 592B is connected to a treatment tool (forceps 420, high-frequency knife 430, local injection needle 440, basket 450, etc.). The treatment tool is inserted through the treatment tool arm 410F via the manipulator flexible portion 417 of the treatment manipulator 400F. The third adaptor 592A and the fourth adaptor 592B have a third drive unit 595.

The third drive unit (driving force transmission part) 595 is a member to which a driving force for driving a treatment tool (forceps 420, high-frequency knife 430, local injection needle 440, basket 450, etc.) is input. The third drive unit 595 is, for example, a rotating drum. The third drive unit 595 has a third coupling portion 595c. The third coupling portion 595c is exposed at the distal end side A1 of the third adaptor 592A and the fourth adaptor 592B.

When the third adaptor 592A and the fourth adaptor 592B are attached to the second motor unit 591, the second coupled portion 593c and the third coupling portion 595c are coupled. As a result, the rotation of the second shaft 593s by the second motor 593m is transmitted to the third drive unit 595. This allows the second drive unit 593 to drive the treatment tools (forceps 420, high-frequency knife 430, local injection needle 440, basket 450, etc.).

The second motor unit 591 has a plurality of second drive units 593 that are assigned to the second adaptor 592, the third adaptor 592A, and the fourth adaptor 592B. The number of second drive units 593 to which the second adaptor 592, the third adaptor 592A, and the fourth adaptor 592B are connected is determined by the number of drive systems for driving the treatment tools, etc.

The second adaptor 592, the third adaptor 592A, and the fourth adaptor 592B each have an identifier such as an RFID. The drive controller 560 can recognize the type of adaptor attached to the second motor unit 591.

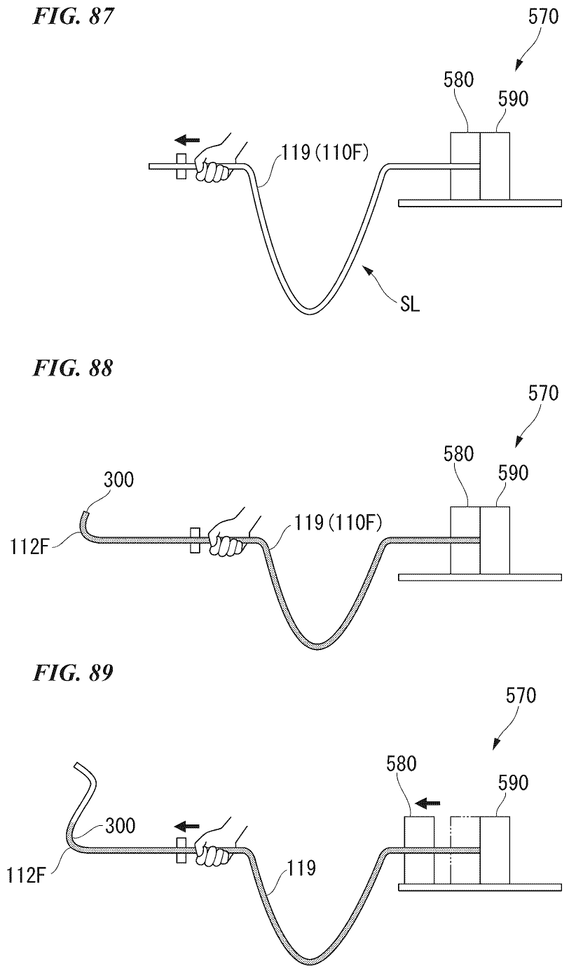

FIGS. 87 to 91 are diagrams showing an example of the operation of the drive unit 570. The surgeon S inserts the insertion section 110F of the insertion manipulator 100F into the patient's large intestine. The hardness-changing device 300 is connected to the second adapter 592 of the second drive unit 590.

As shown in FIG. 87, the surgeon S inserts the insertion section 110F of the insertion manipulator 100F from the distal end into the large intestine from the patient's anus. At this time, the surgeon S provides slack SL in the flexible portion 119 between the part holding the flexible portion 119 and the drive unit 570. The advancement and retraction of the insertion section 110F may be performed by the surgeon S's hand or by the insertion drive unit 540.

As shown in FIG. 88, the surgeon S places the bending portion 112F at a portion that curves significantly in the large intestine. The surgeon S advances the hardness-changing portion 310, whose shape is variable, to place the hardness-changing portion 310 at the bending portion 112F. The surgeon S fixes the shape of the hardness-changing portion 310 that passes through the bending portion 112F.

As shown in FIG. 89, the surgeon S advances the first drive unit 580 toward the distal end side A1 to advance the insertion section 110F. The bending portion 112F advances along the hardness-changing portion 310, the shape of which is fixed. The insertion manipulator 100F can smoothly pass through the large bending portion in the large intestine.

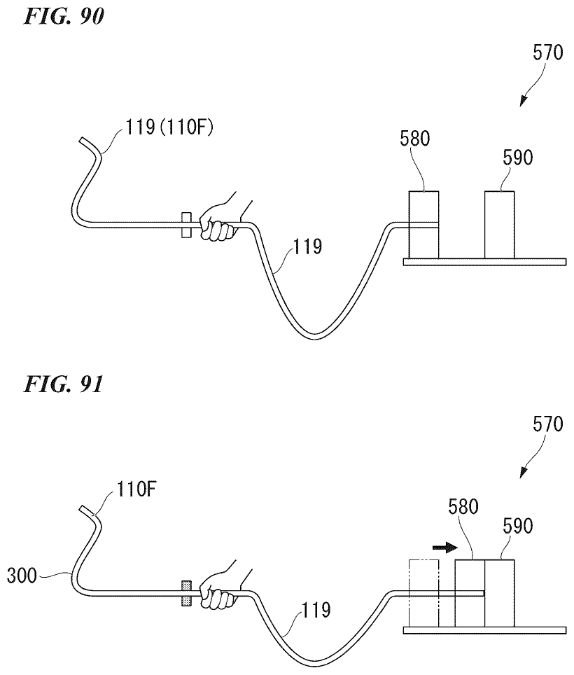

As shown in FIG. 90, the surgeon S releases the wire of the hardness-changing portion 310 to soften the hardness-changing portion 310.

As shown in FIG. 91, the surgeon S holds and fixes the flexible portion 119. Fixing of the insertion section 110F may be performed by the surgeon S's hand or by the insertion drive unit 540. The surgeon S retracts the first drive unit 580 to the proximal end side A2. This reduces the slack SL of the flexible portion 119, and the hardness-changing portion 310 advances.

As described above, by moving the first drive unit 580 back and forth while operating the insertion section 110F, the insertion manipulator 100F can be advanced. Since it is not necessary to keep advancing the first drive unit 580 to advance the insertion manipulator 100F, the movable range of the first drive unit 580 can be limited. The cart 500W on which the drive unit 570 is mounted can be made smaller.

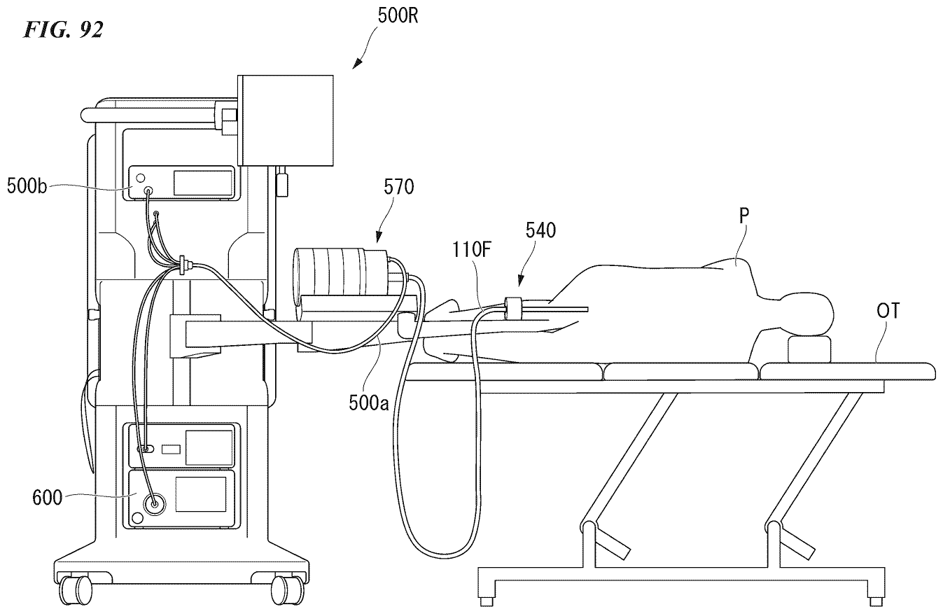

FIG. 92 shows a rack 500R, which is a modified example of the cart 500W.

The drive unit 570 is mounted on an arm 500a that extends deformably from the rack 500R. The drive unit main body 500b and the image control device 600 can be mounted on the rack 500R.

### [Scope 200]

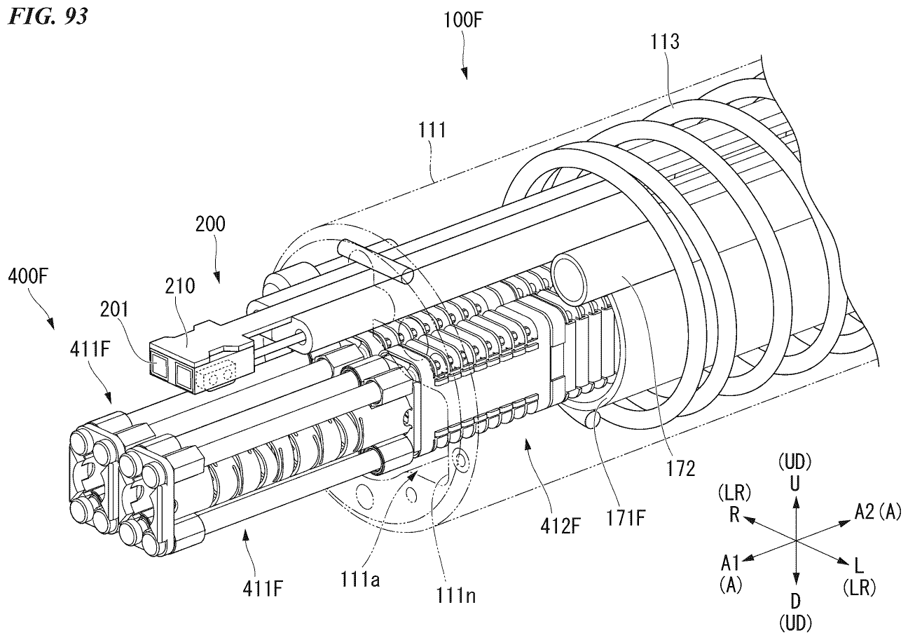

FIG. 93 shows the scope 200 protruding from the distal end 111.

The scope (camera unit) 200 is attached so that it can protrude from the distal end 111 to the distal end side A1 and can be bent. The scope 200 is operated by the scope operation wire 178. The scope (camera unit) 200 includes an imaging unit (camera) 201 and a support portion 220 that supports the imaging unit 201. The imaging unit 201 is provided on the distal end side A1 of the support portion 220.

The support portion 220 of the scope 200 has a leaf spring 210 on the upper side (U side). The leaf spring 210 is formed in a flat plate shape extending in the longitudinal direction A. When the scope operation wire 178 is not driven, the leaf spring allows the scope 200 to maintain a linear shape extending in the longitudinal direction A.

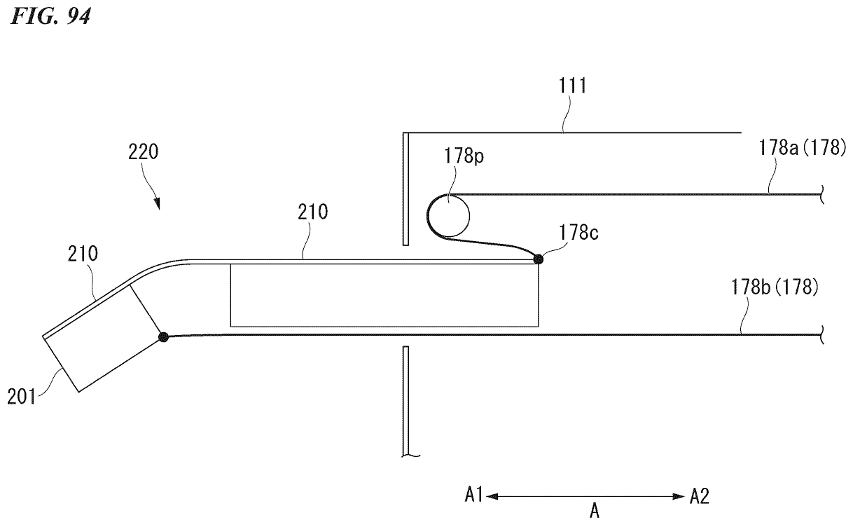

FIG. 94 is a diagram explaining the operation of the scope operation wire 178.

The scope operation wire 178 includes a first scope operation wire 178a and a second scope operation wire 178b.

The first scope operation wire 178a is connected to the proximal end side A2 of the scope 200 via a pulley 178p. The pulley 178p is disposed on the distal end side A1 of the connection part 178c where the first scope operation wire 178a is connected to the scope 200. The pulley 178p reverses the forward and backward direction of the first scope operation wire 178a in the longitudinal direction A.

The distal end of the second scope operation wire 178b is connected to the lower side (D side) of the imaging unit (camera) 201 provided on the distal end side A1 of the scope 200.

The scope 200 transitions between a first state in which the scope 200 advances relative to the distal end 111, a second state in which the scope 200 retracts relative to the distal end 111, and a third state in which the scope 200 bends downward.

When the first scope manipulation wire 178a is pulled, the first scope manipulation wire 178a pulls the connection part 178c toward the distal end side A1, and the scope 200 advances relative to the distal end 111 (first state).

When the second scope manipulation wire 178b is pulled, the scope 200 retracts relative to the distal end 111 (second state).

When the first scope operation wire 178a and the second scope operation wire 178b are pulled, the force pulling the second scope operation wire 178b becomes greater than the holding force of the leaf spring, so that the leaf spring 210 bends downward and the scope 200 bends downward (third state).

The two scope operation wires 178 allow the forward movement, backward movement, and bending of the scope 200 to be controlled, and the distal end 111 can be simplified and made thinner.

The electric endoscope system 1000F according to this embodiment allows observation and treatment to be performed more efficiently.

The fifth embodiment of the present invention has been described above in detail with reference to the drawings, but the specific configuration is not limited to this embodiment and design changes within the scope of the present invention are also included. In addition, the components shown in the above-mentioned embodiments and modifications can be configured in any suitable combination.

### INDUSTRIAL APPLICABILITY

The present invention can be applied to a medical system for observing and treating the inside of a hollow organ, etc.

What is claimed is:

1. A medical manipulator, comprising:
a manipulator; and
a camera unit provided at a distal end of the manipulator so as to be movable back and forth in a longitudinal direction,
wherein the camera unit includes
a support portion, and
a camera provided at a distal end side of the support portion, and
the camera unit includes
a first state in which the camera unit advances relative to the manipulator,
a second state in which the camera unit retracts relative to the manipulator, and
a third state in which the camera unit bends downward.

2. The medical manipulator according to claim 1, wherein
the support portion includes
a bending portion extending in the longitudinal direction,
a first wire connected to a proximal end of the camera unit, and
a second wire connected to an underside of the camera unit, and
the camera unit is
in the first state when the first wire is pulled,
in the second state when the second wire is pulled, and
in the third state when the first wire and the second wire are pulled, with the bending portion bending.

3. The medical manipulator according to claim 2, wherein the first wire is connected via a pulley at the proximal end side of the camera unit.

4. The medical manipulator according to claim 2, wherein the second wire is connected to an underside of the camera.

5. The medical manipulator according to claim 2,
wherein the bending portion includes a leaf spring, and
the camera unit is in the third state when the first wire and the second wire are pulled by bending the leaf spring.

6. The medical manipulator according to claim 1,
wherein the manipulator further includes a channel tube, and
the channel tube is disposed below the camera unit on a distal end surface of the manipulator.

7. The medical manipulator according to claim 6,
wherein the manipulator further includes
an outer sheath, and
a bendable bending portion disposed at the distal end of the outer sheath, and
the camera unit is disposed distal to the bending portion when retracted.

8. The medical manipulator according to claim 7, wherein
the bending portion of the manipulator includes a plurality of ring members through which a bending wire is inserted, and
the camera unit is, in a retracted state, disposed on the distal side of the ring member disposed at the most distal end of the plurality of ring members.

9. The medical manipulator according to claim 8, further comprising a spring formed in a spiral shape along the longitudinal direction,
wherein the outer sheath is disposed outside the spring, and
the channel tube passes through the spring.

10. The medical manipulator according to claim 9, wherein
each of the plurality of ring members has a slit into which the spring is fitted, and
each of the plurality of ring members is attached to the spring by fitting the spring into the slit.

11. A medical manipulator system, comprising:
an insertion manipulator including a manipulator and a camera unit provided at a distal end of the manipulator so as to be movable back and forth in a longitudinal direction;
a drive unit that drives the insertion manipulator; and
a control device that controls the drive unit,
wherein the camera unit includes
a support portion, and
a camera provided at a distal end side of the support portion, and
wherein the camera unit includes
a first state in which the camera unit advances relative to the manipulator,
a second state in which the camera unit retracts relative to the manipulator, and
a third state in which the camera unit bends downward.

12. The medical manipulator system according to claim 11, wherein
the support portion includes
a bending portion extending in the longitudinal direction,
a first wire connected to a proximal end of the camera unit, and
a second wire connected to an underside of the camera unit, and
the camera unit is
in the first state when the first wire is pulled,
in the second state when the second wire is pulled, and
in the third state when the first wire and the second wire are pulled, with the bending portion bending.

13. The medical manipulator system according to claim 12, wherein the first wire is connected via a pulley at the proximal end side of the camera unit.

14. The medical manipulator system according to claim 12, wherein the second wire is connected to an underside of the camera.

15. The medical manipulator system according to claim 12,
wherein the bending portion includes a leaf spring, and
the camera unit is in the third state when the first wire and the second wire are pulled by bending the leaf spring.

16. The medical manipulator system according to claim 11,
wherein the manipulator further includes a channel tube, and
the channel tube is disposed below the camera unit on the distal end surface of the manipulator.

17. The medical manipulator system according to claim 16,
wherein the manipulator includes
an outer sheath, and
a bendable bending portion disposed at a distal end of the outer sheath, and
the camera unit is disposed distal to the bending portion when retracted.

18. The medical manipulator system according to claim 17, wherein
the bending portion of the manipulator includes a plurality of ring members through which a bending wire is inserted, and
the camera unit is, in a retracted state, disposed on the distal side of the ring member disposed at the most distal end of the plurality of ring members.

19. The medical manipulator system according to claim 18, further comprising a spring formed in a spiral shape along the longitudinal direction,
wherein the outer sheath is disposed outside the spring, and
the channel tube passes through the spring.

20. The medical manipulator system according to claim 19, wherein
each of the plurality of ring members includes a slit into which the spring is fitted, and
each of the plurality of ring members is attached to the spring by fitting the spring into the slit.
